(19) United States
(12) Patent Application Publication
(10) Pub. No.: US 2019/0290748 A1
(43) Pub. Date: Sep. 26, 2019

(54) ANTIGENS AND ANTIGEN COMBINATIONS

(71) Applicant: GLAXOSMITHKLINE BIOLOGICALS SA, Rixensart (BE)

(72) Inventors: Marco SORIANI, Siena (IT); Maria SCARSELLI, Siena (IT); Nathalie NORAIS, Rapolano Terme (IT); Danilo GOMES MORIEL, Brisbane (AU); Silvia ROSSI PACCANI, Murlo (IT)

(73) Assignee: GLAXOSMITHKLINE BIOLOGICALS SA, Rixensart (BE)

(21) Appl. No.: 16/363,799

(22) Filed: Mar. 25, 2019

Related U.S. Application Data
(63) Continuation of application No. 14/396,881, filed on Oct. 24, 2014, now Pat. No. 10,279,026.

(30) Foreign Application Priority Data
Apr. 26, 2012 (GB) 1207385.4
Dec. 21, 2012 (EP) 12199079.0

(51) Int. Cl. A61K 39/102; C07K 14/285; C12R 1/21; A61K 39/02; A61K 39/095

(52) U.S. Cl. CPC A61K 39/102; C07K 14/285; A61K 2039/55566; A61K 39/099; A61K 39/095; C12R 1/21

(57) ABSTRACT

NTHI protein antigens have been identified and found to be conserved across several Haemophilus influenzae pathogenic strains. They have been isolated, cloned from a reference strain and tested for immunogenicity. Methods for immunization and vaccines derived thereof are also disclosed.

This application is a Continuation of copending application Ser. No. 14/396,881, filed on Oct. 24, 2014, which is the National Phase under 35 U.S.C. § 371 of International Application No. PCT/EP2013/058459, filed on Apr. 24, 2013, which claims the benefit under 35 U.S.C. § 119(a) to Application No. 1207385.4, filed in Great Britain on Apr. 26, 2012 and Application No. 12199079.0, filed in Europe on Dec. 21, 2012, all of which are hereby expressly incorporated by reference into the present application.

### SEQUENCE LISTING

The instant application contains a Sequence Listing which was submitted electronically in ASCII format in Parent application Ser. No. 14/396,881, and is hereby incorporated by reference in its entirety. Said ASCII copy, created on Mar. 20, 2015, is named PAT054802-US-PCT SL.txt and is 224,185 bytes in size.

### TECHNICAL FIELD

This invention is in the field of Haemophilus influenzae immunology and vaccinology, in particular non-typeable H. influenzae (NTHI). The invention provides antigen polypeptides and combinations of antigen polypeptides for raising antibodies and immune responses and against H. influenzae strains. The invention also provides compositions containing such antigens, and the use thereof as vaccines or medicaments against H. influenzae. The invention also provides immunogenic compositions containing such antigens used alone or in combination or used together with other vaccines. The invention also provides methods for raising immune responses against H. influenzae, and methods for the treatment and prevention of infections by H. influenzae.

### BACKGROUND ART

Haemophilus influenzae is a small, non-motile, Gram-negative coccobacillus. It is a respiratory pathogen that causes a wide spectrum of human infections, including: asymptomatic colonization of the upper respiratory tract (i.e. carriage); infections that extend from colonized mucosal surfaces to cause otitis media (inflammation of the middle ear), bronchitis, conjunctivitis, sinusitis, urinary tract infections and pneumonia; and invasive infections, such as bacteremia, septic arthritis, epiglottitis, pneumonia, empyema, pericarditis, cellulitis, osteomyelitis and meningitis. H. influenzae was the first bacterium for which a complete genome sequence was published [1].

H. influenzae strains are either capsulated (typeable) or non-capsulated (non-typeable), and there are six major serological types of capsulated strains (a to f). 95% of H. influenzae-caused invasive diseases are caused by H. influenzae type b (‘Hib’) strains. The most serious manifestation of Hib disease is meningitis, but the introduction in the 1980s of vaccines based on conjugated Hib capsular saccharides has hugely reduced incidence of this disease.

Although Hib infections can now be controlled by vaccination, other pathogenic H. influenzae strains remain a risk. For instance, non-typeable H. influenzae (NTHI) is responsible for otitis media (OM), particularly chronic and acute OM. While OM is rarely associated with mortality, it is associated with significant morbidity. Hearing loss is the most common complication of OM, with behavioural, educational and language development delays being additional consequences of early onset OM with effusion. Acute OM is the most common bacterial infection in children in the USA. The non-typeable H. influenzae biogroup aegyptius causes epidemic conjunctivitis and Brazilian purpuric fever (BPF) [2], with BPF having a mortality of up to 70%.

To date, antibiotics are the main tool against the spectrum of clinical entities known collectively as OM, but widespread use of antibiotics for OM has met with controversy due to the emergence of multiple-antibiotic resistant microorganisms. Progress towards a vaccine is slow due to an incomplete understanding of both the pathogenesis of OM and the immune response to it.

The genome sequence of the serotype d strain KW20 [1,3] has been useful for understanding basic H. influenzae biology, but it has not been so useful in countering pathogenic H. influenzae strains, as serotype d strains are generally not pathogens. Polypeptides from pathogenic non-typeable H. influenzae have been identified and investigated as vaccine candidates. Reference 4 discloses immunogenic polypeptides from a pathogenic non-typeable H. influenzae strain.

However, there remains a need for providing a vaccine that protects against a broad spectrum of Haemophilus influenzae strains. H. influenzae is a versatile microorganism with an improved ability to adapt to new niches and to cause a broad spectrum of disease. Fitness, virulence and colonization factors can change in order to allow the microorganism to adapt to different tissues and hosts. Therefore, potential antigens are subject to high selective pressure and, as a result, may have sequence variability among different strains.

Thus there remains a need to identify further and improved antigens for use in non-typeable Haemophilus influenzae vaccines, and in particular for vaccines which are useful against multiple NTHI-caused pathologies.

The database of genomes available at ncbi.nlm.nih.gov under genomes listed pathogenic and non-pathogenic Haemophilus influenzae genomes with as few as 2,500 proteins to as many as 4,000 proteins. However, such listings do not identify which are conserved across a significant fraction of the pathogenic NTHI, what are the conserved regions in the proteins that are so conserved, or which proteins among the thousands of potential proteins can be used in a vaccine to produce a sufficient immune response to protect against pathogenic NTHI which requires screening large numbers of proteins to identify the best candidates.

It is an object of the invention to provide further and better antigens and/or combinations which are efficacious in raising immune responses against different strains of H. influenzae, for use in the development of vaccines for preventing and/or treating infections caused by H. influenzae pathogens, in particular non-typeable H. influenzae. In particular, it is an object to provide polypeptides and combinations of polypeptides for use in improved immunogenic compositions and vaccines for preventing and/or in treating such infections, and in particular acute otitis media and chronic obstructive pulmonary disease (COPD). The polypeptides may also be useful for diagnostic purposes, and as targets for antibiotics.

### DISCLOSURE OF THE INVENTION

Present invention describes non-typeable Haemophilus influenzae (NTHI) polypeptides that are useful for immunisation, for use either alone or in combination. These polypeptides may be combined with other NTHI polypeptides as well as. The antigens are useful in NTHI vaccines but may also be used as components in vaccines for immunising against multiple pathogens.

By using two parallel approaches, namely reverse vaccinology and proteomic analysis of outer membrane vesicles (OMVs) it has been possible to identify antigens which are conserved amongst 86 different NTHI strains. Reverse vaccinology uses in silico analysis to identify proteins conserved in the genomes of different NTHI strains and potentially surface-exposed. The second approach is instead focused on the identification of antigens by analysing mass spectrometry of the proteins contained in the outer membrane vesicles produced by NTHI.

The genome of a NTHI strain includes about 1800 genes. The inventors have identified 274 conserved antigens from 15 complete genomes plus 39 strains selected on the basis of geographical distribution and 32 strains derived from an otitis media Finnish collection which are all currently publicly available. From these 274 the inventors have selected 53 polypeptides of particular interest. These antigens were selected from the strain NP86-028, with the exception of CGSHiGG_00130 being selected from PittG, CGSHiGG_02400 selected from PittG, gi-145633184 selected from 3655 strain and gi-145628236 selected from 22.1-21 strain.

Amongst the group of 53 antigens the following further selection has been generated considering immunogenicity and conservation criteria:
A set of 26 antigens referred herein as “the first antigen group”
A set of 6 antigens referred herein as “the second antigen group”
A set of 21 antigens referred herein as “the third antigen group”

Most preferred set of antigens is referred to herein as ‘the first antigen group’. Thus the invention provides an immunogenic composition comprising at least one antigen, preferably comprising one or more (i.e. 1, 2, 3, 4, 5, 6 or more) antigens selected from the group consisting of: (1) NTHI0915 (NT018), (2) NTHI1416 (NT024), (3) NTHI2017 (NT032), (4) CGSHiGG_02400 (NT038), (5) NTHI1292 (NT067), (6) NTHI0877 (NT001), (7) NTHI0266 (NT016), (8) CGSHiGG_00130 (NT052), (9) NTHI1627 (NT002), (10) NTHI1109 (NT026), (11) NTHI0821 (NT009), (12) NTHI0409 (NT025), (13) NTHI1954 (NT028), (14) NTHI0371 (NT029), (15) NTHI0509 (NT031), (16) NTHI0449 (NT015), (17) NTHI1473 (NT023), (18) gi-145633184 (NT100), (19) NTHI1110 (NT040), (20) gi-46129075 (NT048), (21) gi-145628236 (NT053), (22) NTHI1230 (NT066), (23) NTHI0522 (NT097), (24) NT004, (25) NT014, (26) NT022. These antigens show a positive bactericidal activity as shown in Table III and Table IV.

Within the first antigen group, preferred antigens are selected from a subset of any of (1) NTHI0915 (NT018) antigen, (2) NTHI1416 (NT024) antigen, (3) NTHI2017 (NT032) antigen, (4) CGSHiGG_02400 (NT038), (5) NTHI1292 antigen (NT067), (6) NTHI0877 (NT001) antigen, (8) NT052 antigen, (24) NT004 antigen, (25) NT014 antigen, (26) NT022 antigen, (7) NTHI0266 NT016 antigen. These antigens are all showing a good level of purification as shown in Table II and immunogenicity efficacy is reported in tables III and IV.

Particularly preferred antigens were NT067, NT014, NT016, NT022.

Thus the invention provides an immunogenic composition comprising one or more (i.e. 1, 2, 3, 4, 5, 6 or more) antigens selected from the group consisting from the “first antigen group”.

The inventors have also identified the following 6 polypeptides: (24) P48 (NTHI0254 also defined as NT007), (25) HtrA (NTHI1905 also defined as NT006), (26) PE (NTHI0267 also defined as NT035), (27) P26 (NTHI0501 also defined as NT010), (28) PHiD (NTHI0811 also defined as NT080), (29) P6 (NTHI0501, also defined as NT081). This set of 6 antigens is referred to herein as ‘the second antigen group’.

The inventors have also identified the following 22 polypeptides: (30) NTHI0532 (NT013), (31) NTHI0363 (NT106), (32) NTHI0370 (NT107), (33) NTHI0205 (NT108), (34) NTHI0374 (NT109), (35) NTHI0579 (NT110), (36) NTHI0837 (NT111), (37) NTHI0849 (NT112), (38) NTHI0921 (NT113), (39) NTHI0995 (NT114), (40) NTHI1091 (NT115), (41) NTHI1169 (NT116), (42) NTHI1208 (NT117), (43) NTHI1318 (NT118), (44) NTHI1796 (NT123), (45) NTHI1930 (NT124), (46) NTHI1565 (NT119), (47) NTHI1569 (NT120), (48) NTHI1571 (NT121), (49) NTHI1667 (NT122), (50) NTHI0588 (NT061), (51) NTHI0915 (NT017). This set of 22 antigens is referred to herein as ‘the third antigen group’.

In one embodiment, a composition includes at least one antigen (i.e. 1, 2, 3, 4, 5, 6 or more) selected from the first antigen group and/or at least one antigen (i.e. 1, 2, 3, 4, 5, 6 or more) selected from the second antigen group and/or at least one antigen (i.e. 1, 2, 3, 4, 5, 6 or more) selected from the third antigen group. Antigens from the first antigen group can be selected from the most preferred subset of antigens.

Preferably the invention provides an immunogenic composition comprising one antigen selected from any of the first antigen group or second antigen group or third antigen group.

Thus the invention also provides an immunogenic composition comprising a combination of antigens, said combination comprising two or more (i.e. 2, 3, 4, 5, 6 or more) antigens selected from the group consisting of the “first antigen group” and/or the “second antigen group” and/or the “third antigen group”.

Where a composition includes an antigen from the “second antigen group”, it is preferred that the composition should also include (i) at least one further antigen from the “second antigen group” or (ii) at least one antigen from the “first antigen group” or the “third antigen group”. Thus the invention would not encompass a composition including as its sole antigenic component a single antigen from the “second antigen group”. Where a composition includes two or more antigens from the “second antigen group”, it is preferred that the composition should include at least one antigen which is not (a) a P48 antigen (b) a HtrA antigen (c) a PE antigen or (d) a P26 antigen. Thus in some embodiments the invention does not encompass combinations only of P48, HtrA, PE and/or P26. Similarly, in some embodiments the invention does not encompass hybrid antigens which include ‘X’ moieties only from P48, HtrA, PE and/or P26.

Within the 11 preferred antigens of the first antigen group there are 55 possible pairs of different antigens. All such pairs are disclosed herein and are part of the invention. Thus the invention provides an immunogenic composition comprising a pair of antigens, wherein said pair is one of said 55 pairs.

In one embodiment, a composition includes at least one antigen (i.e. 1, 2, 3, 4, 5, 6 or more) selected from the first antigen group and/or at least one antigen (i.e. 1, 2, 3, 4, 5, 6 or more) selected from the second antigen group, and/or at least one antigen (i.e. 1, 2, 3, 4, 5, 6 or more) selected from the third antigen group.

In all cases, antigens from the first antigen group can be advantageously selected from the most preferred subset of any of (1) NTHI0915 (NT018), (2) NTHI1416 (NT024), (3) NTHI2017 (NT032), (4) CGSHiGG_02400 (NT038), (5) NTHI1292 (NT067), (6) NTHI0877 (NT001), (8) NT052, (24) NT004, (25) NT014, (26) NT022, (7) NT016.

The invention also provides an immunogenic composition comprising a combination of antigens, said combination comprising two or more (i.e. 2, 3, 4 or 5) antigens selected from the group consisting of: (1) NTHI0915 (NT018), (2) NTHI1416 (NT024), (3) NTHI2017 (NT032), (4) CGSHiGG_02400 (NT038), (5) NTHI1292 (NT067), (6) NTHI0877 (NT001), (8) NT052, (24) NT004, (25) NT014, (26) NT022, (7) NT016. The composition can also include an adjuvant e.g. an adjuvant comprising an oil-in-water emulsion or an aluminium salt.

Reference 5 discusses non-typeable H. influenzae antigens, inter alia as candidates for potential use in vaccines. References 6 to 10, are concerned, individually, with non-typeable H. influenzae polypeptides P48, HtrA, PE and P26, respectively, and inter alia with their immunogenic potential. Reference 5 also mentions HtrA, PE and P26 individually amongst a larger number of vaccine candidates, and e.g. reference 10 is concerned with polypeptide PE. However, these antigens, belonging to the “second antigen group” and were not described for use in combination. It has now surprisingly been found that a combination of one or more of these antigens (second antigen group) with at least one of the antigen listed in the “first antigen group” is particularly suitable for generating a protective immune response against non-typeable H. influenzae, and thus the above-mentioned objects of the invention.

Advantageous combinations of the invention are those in which two or more antigens act synergistically. Thus the protection against NTHI pathogen achieved by their combined administration exceeds that expected by mere addition of their individual protective efficacy.

First Antigen Group

NT018 Antigen

The “NT018” antigen is annotated as TPR repeat-containing protein and also as cytochrome c maturation heme lyase subunit CcmH2. It has been annotated as NTHI0915 in the strain 86-028NP. Said sequence is highly conserved amongst all the strains analyzed and is predicted to be a membrane-bound metal-peptidase. NT018 is surface exposed as shown in Table III. NT018 has been cloned and expressed from another non-typeable strain, Fi176, which is a strain isolated form the Finland otitis media collection.

Useful NT018 antigens can elicit an antibody (e.g. when administered to a human) that recognises SEQ ID NO: 1 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 1; and/or (b) comprising a fragment of at least ‘n’ consecutive amino acids of SEQ ID NO: 1, wherein ‘n’ is 7 or more (e.g. 8, 10, 12, 14, 16, 18, 20, 25, 30, 35, 40, 50, 60, 70, 80, 90, 100, 150, 200, 250 or more). These NT018 proteins include variants of SEQ ID NO: 1, such as SEQ ID NO: 49 which has been cloned and expressed and tested in immunogenicity (Table III, IV). Preferred fragments of (b) comprise an epitope from SEQ ID NO: 1. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25, 26, 27, 28 or more) from the N-terminus of SEQ ID NO: 1 while retaining at least one epitope of SEQ ID NO: 1. Other fragments omit one or more protein domains.

A NT018 antigen of the invention can be expressed with its native 28 N-terminal amino acids of NT018 (MNFTLIFILTTLVVALICFYPLLRQFKA; SEQ ID NO: 69) or can be expressed with an alternative N-terminal sequence e.g. with a simple N-terminus methionine, or Met-Ala-, or a leader peptide which targets or traffics the expressed protein in a desired fashion.

NT024 Antigen

The “NT024” antigen is annotated as “hypothetical protein” and has been annotated as NTHI1416 in the genome 86-028NP. This antigen has been cloned and expressed from Fi176 strain.

Useful NT024 antigens can elicit an antibody (e.g. when administered to a human) that recognises SEQ ID NO: 2 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 2; and/or (b) comprising a fragment of at least ‘n’ consecutive amino acids of SEQ ID NO: 2 wherein ‘n’ is 7 or more (e.g. 8, 10, 12, 14, 16, 18, 20, 25, 30, 35, 40, 50, 60, 70, 80, 90, 100, 150, 200, 250 or more). These NT024 proteins include variants of SEQ ID NO: 2, such as SEQ ID NO: 50 cloned from strain Fi176. Preferred fragments of (b) comprise an epitope from SEQ ID NO: 2. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 2 while retaining at least one epitope of SEQ ID NO: 2. Other fragments omit one or more protein domains.

A NT024 antigen of the invention can be expressed with the native 20 N-terminal amino acids of NT024 (MKLKLFFHIVLLCFSLPVWA; SEQ ID NO: 70) or can be expressed with an alternative N-terminal sequence e.g. with a simple N-terminus methionine, or Met-Ala-, or a leader peptide which targets or traffics the expressed protein in a desired fashion.

NT032 Antigen

The “NT032” antigen is annotated as “hypothetical protein” and has been annotated as NTHI12017 in the genome 86-028NP. Domain most conserved amongst strains tested is described as “Bacterial OB fold (BOF) protein”. This antigen has been cloned and expressed from Fi176 strain.

Useful NT032 antigens can elicit an antibody (e.g. when administered to a human) that recognises SEQ ID NO: 3 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 3; and/or (b) comprising a fragment of at least ‘n’ consecutive amino acids of SEQ ID NO: 3, wherein ‘n’ is 7 or more (e.g. 8, 10, 12, 14, 16, 18, 20, 25, 30, 35, 40, 50, 60, 70, 80, 90, 100, 150, 200, 250 or more). These NT032 proteins include variants of SEQ ID NO: 3, such as SEQ ID NO: 51 cloned from Fi176 strain. Preferred fragments of (b) comprise an epitope from SEQ ID NO: 3. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 3 while retaining at least one epitope of SEQ ID NO: 3. Other fragments omit one or more protein domains.

A NT032 antigen of the invention can be expressed with the native 19 N-terminal amino acids of NT032 (MKKFALATIFALATTSAFA; SEQ ID NO: 71) or can be expressed with an alternative N-terminal sequence e.g. with a simple N-terminus methionine, or Met-Ala-, or a leader peptide which targets or traffics the expressed protein in a desired fashion.

NT067 Antigen

The “NT067” antigen is annotated as ABC transporter protein and it has been proposed its hypothetical function as periplasmic oligopeptide-binding protein OppA. In the strain 86-028NP has been annotated as NTHI1292. This antigen has been cloned and expressed from Fi176 strain.

Useful NT067 antigens can elicit an antibody (e.g. when administered to a human) that recognises SEQ ID NO: 5 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 5; and/or (b) comprising a fragment of at least ‘n’ consecutive amino acids of SEQ ID NO: 5, wherein ‘n’ is 7 or more (e.g. 8, 10, 12, 14, 16, 18, 20, 25, 30, 35, 40, 50, 60, 70, 80, 90, 100, 150, 200, 250 or more). These NT067 proteins include variants of SEQ ID NO: 5, such as SEQ ID NO: 52 cloned from Fi176 strain. Preferred fragments of (b) comprise an epitope from SEQ ID NO: 5. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 5 while retaining at least one epitope of SEQ ID NO: 5. Other fragments omit one or more protein domains.

A NT067 antigen of the invention can be expressed with the native 20 N-terminal amino acids of NT067 (MQHKLLFSAIALALSYSVQA; SEQ ID NO: 72) or can be expressed with an alternative N-terminal sequence e.g. with a simple N-terminus methionine, or Met-Ala-, or a leader peptide which targets or traffics the expressed protein in a desired fashion.

NT038 Antigen

This antigen is known as Hia (Haemophilus influenzae adhesin) protein [11] and has been identified in the strain CGSHiGG_02400 as a 282 aa in length, however it is a truncated form of Hia (616 aa) as originally described in the strain 86-028NP or in other NTHi strains. This antigen has been cloned from R2846 strain.

Useful NT038 antigens can elicit an antibody (e.g. when administered to a human) that recognises SEQ ID NO: 4 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 4; and/or (b) comprising a fragment of at least ‘n’ consecutive amino acids of SEQ ID NO: 4, wherein ‘n’ is 7 or more (e.g. 8, 10, 12, 14, 16, 18, 20, 25, 30, 35, 40, 50, 60, 70, 80, 90, 100, 150, 200, 250 or more). These NT038 proteins include variants of SEQ ID NO: 4, such as SEQ ID NO: 53, which is lacking the first 23 native N-terminal amino acids and 102 amino acids at the C-terminal. Preferred fragments of (b) comprise an epitope from SEQ ID NO: 4. Other preferred fragments lack one or more amino acids (e.g. 1, 2, 3, 4, 5, 6, 7, 8, 9, 10, 15, 20, 25 or more) from the C-terminus (even up to 102aa) and/or one or more amino acids (e.g. 1, 2, 3, 4, 5, 6, 7, 8, 9, 10, 15, 20, 23, 25 or more) from the N-terminus of SEQ ID NO: 4 while retaining at least one epitope of SEQ ID NO: 4. Other fragments omit one or more protein domains.

A NT038 antigen of the invention can be expressed with the native 23 N-terminal amino acids of NT038 (MPFQYVTEDGKTVVKVGNGYYEA; SEQ ID NO: 73) or can be expressed with an alternative N-terminal sequence e.g. with a simple N-terminus methionine, or Met-Ala-, or a leader peptide which targets or traffics the expressed protein in a desired fashion.

NT001 Antigen

This antigen has been annotated as NTHI0877 in the genome 86-028NP and is known as D-methionine-binding lipoprotein MetQ. MetD is an ABC transporter encoding a DL methionine uptake system. This antigen has been previously disclosed as BASB202 (28 Kda) [12, 13], and its use as vaccine against NTHI has been proposed. This antigen shares 99.63% alignment ID with an homologue antigen as described in Ref (4) and it has been found well conserved amongst all the strains considered in the present invention. In present invention it is cloned and expressed from Fi176 strain.

Useful NT001 antigens can elicit an antibody (e.g. when administered to a human) that recognises SEQ ID NO: 6 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 6; and/or (b) comprising a fragment of at least ‘n’ consecutive amino acids of SEQ ID NO: 6, wherein ‘n’ is 7 or more (e.g. 8, 10, 12, 14, 16, 18, 20, 25, 30, 35, 40, 50, 60, 70, 80, 90, 100, 150, 200, 250 or more). These NT001 proteins include variants of SEQ ID NO: 4, such as SEQ ID NO: 54. Preferred fragments of (b) comprise an epitope from SEQ ID NO: 6. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 21, 25 or more) from the N-terminus of SEQ ID NO: 6 while retaining at least one epitope of SEQ ID NO: 6. Other fragments omit one or more protein domains.

A NT001 antigen of the invention can be expressed with the native 21 N-terminal amino acids of NT001 (MKLKQLFAITAIASALVLTGC; SEQ ID NO: 74) or can be expressed with an alternative N-terminal sequence e.g. with a simple N-terminus methionine, or Met-Ala-, or a leader peptide which targets or traffics the expressed protein in a desired fashion.

NT016 Antigen

This antigen has been annotated as NTHI0266 in the strain 86-028NP and described as Hypothetical lipoprotein. This antigen has been cloned and expressed from Fi176 strain.

Useful NT016 antigens can elicit an antibody (e.g. when administered to a human) that recognises SEQ ID NO: 7 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 7; and/or (b) comprising a fragment of at least ‘n’ consecutive amino acids of SEQ ID NO: 7, wherein ‘n’ is 7 or more (e.g. 8, 10, 12, 14, 16, 18, 20, 25, 30, 35, 40, 50, 60, 70, 80, 90, 100, 150, 200, 250 or more). These NT016 proteins include variants of SEQ ID NO: 7, such as SEQ ID NO: 55. Preferred fragments of (b) comprise an epitope from SEQ ID NO: 7. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 19, 20, 25 or more) from the N-terminus of SEQ ID NO: 7 while retaining at least one epitope of SEQ ID NO: 7. Other fragments omit one or more protein domains.

A NT016 antigen of the invention can be expressed with the native 16 N-terminal amino acids of NT016 (MRKIKSLALLAVAALVIGC; SEQ ID NO: 75) or can be expressed with an alternative N-terminal sequence e.g. with a simple N-terminus methionine, or Met-Ala-, or a leader peptide which targets or traffics the expressed protein in a desired fashion.

NT052 Antigen

This antigen has been annotated as CGSHiGG_00130 from PittGG strain. It is part of Sell-domain containing protein families. It has been cloned from R2846 strain and the cloned sequence is reported as SEQ ID NO: 8. Despite the sequence cloned from R2846 is sharing only 64.16% identity over the sequence as annotated CGSHiGG_00130, it has been shown that there are conserved Sell domains which are repeated along the sequence which are useful to provide an efficacious antigenicity. Consensus for this repeats is SEQ ID NO: EAVKWYRKAAEQ.

Useful NT052 antigens can elicit an antibody (e.g. when administered to a human) that recognises SEQ ID NO: 8 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 8; and/or (b) comprising a fragment of at least ‘n’ consecutive amino acids of SEQ ID NO: 8, wherein ‘n’ is 7 or more (e.g. 8, 10, 12, 14, 16, 18, 20, 25, 30, 35, 40, 50, 60, 70, 80, 90, 100, 150, 200, 250 or more). These NT052 proteins include variants of SEQ ID NO: 8, such as SEQ ID NO: 56. Preferred fragments of (b) comprise an epitope from SEQ ID NO: 8. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 11, 15, 20, 25 or more) from the N-terminus of SEQ ID NO: 8 while retaining at least one epitope of SEQ ID NO: 8. Other fragments omit one or more protein domains.

A NT052 antigen of the invention can be expressed with the native 11 N-terminal amino acids of NT052 (MLLFILSIAWA; SEQ ID NO: 76) or can be expressed with an alternative N-terminal sequence e.g. with a simple N-terminus methionine, or Met-Ala-, or a leader peptide which targets or traffics the expressed protein in a desired fashion.

NT002 Antigen

This antigen has been annotated as NTHI1627 in 86-026NP strain and as lipoprotein. It has been cloned and expressed from Fil76 strain.

Useful NT002 antigens can elicit an antibody (e.g. when administered to a human) that recognises SEQ ID NO: 9 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) comprising a fragment of at least ‘n’ consecutive amino acids of SEQ ID NO: 9, wherein ‘n’ is 7 or more (e.g. 8, 10, 12, 14, 16, 18, 20, 25, 30, 35, 40, 50, 60, 70, 80, 90, 100, 150, 200, 250 or more). These NT002 proteins include variants of SEQ ID NO: 9, such as SEQ ID NO: 57. Preferred fragments of (b) comprise an epitope from SEQ ID NO: 9. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 9 while retaining at least one epitope of SEQ ID NO: 9. Other fragments omit one or more protein domains.

A NT002 antigen of the invention can be expressed with the native 18 N-terminal amino acids of NT002 (MKVYKSFLIATASLFLFA; SEQ ID NO: 77) or can be expressed with an alternative N-terminal sequence e.g. with a simple N-terminus methionine, or Met-Ala-, or a leader peptide which targets or traffics the expressed protein in a desired fashion.

A NT002 antigen of the invention can be a lipoprotein e.g. lipidated at a N-terminus cysteine.

NT026 Antigen

This antigen has been annotated as hypothetical protein NTHI1109 in 86-026NP strain. It has been predicted to be a cytoplasmic membrane protein. It has been cloned and expressed from strain Fi176.

Useful NT026 antigens can elicit an antibody (e.g. when administered to a human) that recognises SEQ ID NO: 10 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO:10 wherein ‘n’ is 7 or more (e.g. 8, 10, 12, 14, 16, 18, 20, 25, 30, 35, 40, 50, 60, 70, 80, 90, 100, 150, 200, 250 or more). These NT026 proteins include variants of SEQ ID NO: 10, such as SEQ ID NO: 58, cloned from Fi176 strain. Preferred fragments of (b) comprise an epitope from SEQ ID NO: 10. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 10 while retaining at least one epitope of SEQ ID NO: 10. Other fragments omit one or more protein domains.

A NT026 antigen of the invention can be expressed with the native 24 N-terminal amino acids of NT026 (MQKGMTLVELLIGLAIISIVLNFA; SEQ ID NO: 78) or can be expressed with an alternative N-terminal sequence e.g. with a simple N-terminus methionine, or Met-Ala-, or a leader peptide which targets or traffics the expressed protein in a desired fashion.

NT009 Antigen

This antigen has been annotated as NTHI0821 in 86-026NP strain and is part of OMP85 family protein. It is located in the outer membrane of the bacteria. It has been cloned and expressed from Fi176 strain. Useful NT009 antigens can elicit an antibody (e.g. when administered to a human) that recognises SEQ ID NO: 11 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). These NT009 proteins include variants of SEQ ID NO: 11, such as SEQ ID NO: 59 cloned from Fi176. Preferred fragments of (b) comprise an epitope from SEQ ID NO: 11. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 22, 25 or more) from the N-terminus of SEQ ID NO: 11 while retaining at least one epitope of SEQ ID NO: 11. Other fragments omit one or more protein domains.

A NT009 antigen of the invention can be expressed with the native 22 N-terminal amino acids of NT009 (MNKTLLKLTALFLALNCFPAFA; SEQ ID NO: 79) or can be expressed with an alternative N-terminal sequence e.g. with a simple N-terminus methionine, or Met-Ala-, or a leader peptide which targets or traffics the expressed protein in a desired fashion.

NT025 Antigen

This antigen has been annotated as NTHI0409 in 86-026NP strain and belongs to the type IV pilin subunit protein family. It has been cloned and expressed from Fi176 strain.

Useful NT025 antigens can elicit an antibody (e.g. when administered to a human) that recognises SEQ ID NO: 12 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). These NT025 proteins include variants of SEQ ID NO: 12, such as SEQ ID NO: 60 as cloned from Fi176. Preferred fragments of (b) comprise an epitope from SEQ ID NO: 12. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 23, 25 or more) from the N-terminus of SEQ ID NO: 12 while retaining at least one epitope of SEQ ID NO: 12. Other fragments omit one or more protein domains.

A NT025 antigen of the invention can be expressed with the native 23 N-terminal amino acids of NT025 (MKLTTQQTLKKGFTLIELMIVIA; SEQ ID NO: 80) or can be expressed with an alternative N-terminal sequence e.g. with a simple N-terminus methionine, or Met-Ala-, or a leader peptide which targets or traffics the expressed protein in a desired fashion.

NT028 Antigen

This antigen has been annotated as NTHI1954 in 86-026NP strain and as lipoprotein NlpC. It has been cloned and expressed from Fi176 strain.

Useful NT028 antigens can elicit an antibody (e.g. when administered to a human) that recognises SEQ ID NO: 13 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) comprising a fragment of at least ‘n’ consecutive amino acids of SEQ ID NO: 13, wherein ‘n’ is 7 or more (e.g. 8, 10, 12, 14, 16, 18, 20, 25, 30, 35, 40, 50, 60, 70, 80, 90, 100, 150, 200, 250 or more). These NT028 proteins include variants of SEQ ID NO: 13, such as SEQ ID NO: 61 as cloned from Fi176. Preferred fragments of (b) comprise an epitope from SEQ ID NO: 13. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 13 while retaining at least one epitope of SEQ ID NO: 13. Other fragments omit one or more protein domains.

A NT028 antigen of the invention can be expressed with the native 21 N-terminal amino acids of NT028 (MLKRILVIIGLAVLATACSNA; SEQ ID NO: 81) or can be expressed with an alternative N-terminal sequence e.g. with a simple N-terminus methionine, or Met-Ala-, or a leader peptide which targets or traffics the expressed protein in a desired fashion.

A NT028 antigen of the invention can be a lipoprotein e.g. lipidated at a N-terminus cysteine.

NT029 Antigen

This antigen has been annotated as NTHI10371 in 86-026NP strain and as heme/hemopexin binding protein A, belonging to the outer membrane protein family. It has been cloned and expressed from R2846 strain.

Useful NT029 antigens can elicit an antibody (e.g. when administered to a human) that recognises SEQ ID NO: 14 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). These NT029 proteins include variants of SEQ ID NO: 14, such as SEQ ID NO 62 cloned from R2846 strain. Preferred fragments of (b) comprise an epitope from SEQ ID NO: 14. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 14 while retaining at least one epitope of SEQ ID NO: 14. Other fragments omit one or more protein domains.

A NT029 antigen of the invention can be expressed with the native 21 N-terminal amino acids of NT029 (MYKLNVISLIILTTYTGATYA; SEQ ID NO: 82) or can be expressed with an alternative N-terminal sequence e.g. with a simple N-terminus methionine, or Met-Ala-, or a leader peptide which targets or traffics the expressed protein in a desired fashion.

NT031 Antigen

This antigen has been annotated as starvation inducible outer membrane lipoprotein NTHI0509 in 86-026NP strain. It has been cloned and expressed from R2846 strain.

Useful NT031 antigens can elicit an antibody (e.g. when administered to a human) that recognises SEQ ID NO: 15 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 15; and/or (b) comprising a fragment of at least ‘n’ consecutive amino acids of SEQ ID NO: 15, wherein ‘n’ is 7 or more (e.g. 8, 10, 12, 14, 16, 18, 20, 25, 30, 35, 40, 50, 60, 70, 80, 90, 100, 150, 200, 250 or more). These NT031 proteins include variants of SEQ ID NO: 15, such as SEQ ID NO: 63 cloned and expressed from R2846 strain. Preferred fragments of (b) comprise an epitope from SEQ ID NO: 15. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 18, 20, 25 or more) from the N-terminus of SEQ ID NO: 15 while retaining at least one epitope of SEQ ID NO: 15. Other fragments omit one or more protein domains.

A NT031 antigen of the invention can be expressed with the native 18 N-terminal amino acids of NT031 (MKGKITLFFTALCFGLTG; SEQ ID NO: 83) or can be expressed with an alternative N-terminal sequence e.g. with a simple N-terminus methionine, or Met-Ala-, or a leader peptide which targets or traffics the expressed protein in a desired fashion.

A NT031 antigen of the invention can be a lipoprotein e.g. lipidated at a N-terminus cysteine.

NT015 Antigen

This antigen has been annotated as opacity associated protein OapB NTHI0449 in 86-026NP strain. It has been cloned and expressed from Fi176 strain.

Useful NT015 antigens can elicit an antibody (e.g. when administered to a human) that recognises SEQ ID NO: 16 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 16; and/or (b) comprising a fragment of at least ‘n’ consecutive amino acids of SEQ ID NO: 16, wherein ‘n’ is 7 or more (e.g. 8, 10, 12, 14, 16, 18, 20, 25, 30, 35, 40, 50, 60, 70, 80, 90, 100, 150, 200, 250 or more). These NT015 proteins include variants of SEQ ID NO: 16, such as SEQ ID NO: 64 cloned and expressed from Fi176. Preferred fragments of (b) comprise an epitope from SEQ ID NO: 16. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 16 while retaining at least one epitope of SEQ ID NO: 16. Other fragments omit one or more protein domains.

A NT015 antigen of the invention can be expressed with the native 17 N-terminal amino acids of NT015 (MLKKTSLIFTALLLAGC; SEQ ID NO: 84) or can be expressed with an alternative N-terminal sequence e.g. with a simple N-terminus methionine, or Met-Ala-, or a leader peptide which targets or traffics the expressed protein in a desired fashion.

NT023 Antigen

This antigen has been annotated as outer membrane lipoprotein PCP, NTHI1473 in 86-026NP strain. It has been cloned and expressed from Fi176 strain.

Useful NT023 antigens can elicit an antibody (e.g. when administered to a human) that recognises SEQ ID NO: 17 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 17; and/or (b) comprising a fragment of at least ‘n’ consecutive amino acids of SEQ ID NO: 17, wherein ‘n’ is 7 or more (e.g. 8, 10, 12, 14, 16, 18, 20, 25, 30, 35, 40, 50, 60, 70, 80, 90, 100, 150, 200, 250 or more). These NT023 proteins include variants of SEQ ID NO: 17, such as SEQ ID NO: 65 cloned and expressed from strain Fi176. Preferred fragments of (b) comprise an epitope from SEQ ID NO: 17. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 17 while retaining at least one epitope of SEQ ID NO: 17. Other fragments omit one or more protein domains.

A NT023 antigen of the invention can be expressed with the native 20 N-terminal amino acids of NT023 (MKKTNMALALLVAFSVTGCA; SEQ ID NO: 85) or can be expressed with an alternative N-terminal sequence e.g. with a simple N-terminus methionine, or Met-Ala-, or a leader peptide which targets or traffics the expressed protein in a desired fashion.

A NT023 antigen of the invention can be a lipoprotein e.g. lipidated at a N-terminus cysteine.

NT100 Antigen

This antigen has been annotated as “putative hydroxamate-type ferric siderophore receptor” and in NCBI as gi-145633184 from strain 3655. It has been cloned from 8246 strain.

Useful NT100 antigens can elicit an antibody (e.g. when administered to a human) that recognises SEQ ID NO: 18 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 18; and/or (b) comprising a fragment of at least ‘n’ consecutive amino acids of SEQ ID NO: 18, wherein ‘n’ is 7 or more (e.g. 8, 10, 12, 14, 16, 18, 20, 25, 30, 35, 40, 50, 60, 70, 80, 90, 100, 150, 200, 250 or more). These NT100 proteins include variants of SEQ ID NO: 18, such as SEQ ID NO: 66. Preferred fragments of (b) comprise an epitope from SEQ ID NO: 18. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 18 while retaining at least one epitope of SEQ ID NO: 18. Other fragments omit one or more protein domains.

A NT100 antigen of the invention can be expressed with the native 30 N-terminal amino acids of NT100 (MDLGPIYNTRDINDGKVINIDNPNYTNPVA; SEQ ID NO: 86) or can be expressed with an alternative N-terminal sequence e.g. with a simple N-terminus methionine, or Met-Ala-, or a leader peptide which targets or traffics the expressed protein in a desired fashion.

NT040 Antigen

This antigen has been annotated as hypothetical protein NTHI1110 in 86-026NP strain. It has been cloned and expressed from R2846 strain

Useful NT040 antigens can elicit an antibody (e.g. when administered to a human) that recognises SEQ ID NO: 19 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 19; and/or (b) comprising a fragment of at least ‘n’ consecutive amino acids of SEQ ID NO: 19, wherein ‘n’ is 7 or more (e.g. 8, 10, 12, 14, 16, 18, 20, 25, 30, 35, 40, 50, 60, 70, 80, 90, 100, 150, 200, 250 or more). These NT040 proteins include variants of SEQ ID NO: 19. Preferred fragments of (b) comprise an epitope from SEQ ID NO: 19. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 19 while retaining at least one epitope of SEQ ID NO: 19. Other fragments omit one or more protein domains.

A NT040 antigen of the invention can be expressed with the native 26 N-terminal amino acids of NT040 (MMKTLLKGQTLLALMISLTLSSLLLL; SEQ ID NO: 87) or can be expressed with an alternative N-terminal sequence e.g. with a simple N-terminus methionine, or Met-Ala-, or a leader peptide which targets or traffics the expressed protein in a desired fashion.

NT048 Antigen

This antigen has been annotated as NTHI1169 in strain 86-028NP. It has been cloned and expressed from R2846 strain.

Useful NT048 antigens can elicit an antibody (e.g. when administered to a human) that recognises SEQ ID NO: 20 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 20; and/or (b) comprising a fragment of at least ‘n’ consecutive amino acids of SEQ ID NO: 20, wherein ‘n’ is 7 or more (e.g. 8, 10, 12, 14, 16, 18, 20, 25, 30, 35, 40, 50, 60, 70, 80, 90, 100, 150, 200, 250 or more). These NT048 proteins include variants of SEQ ID NO: 20. Preferred fragments of (b) comprise an epitope from SEQ ID NO: 20. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 20 while retaining at least one epitope of SEQ ID NO: 20. Other fragments omit one or more protein domains. A NT048 antigen of the invention can be expressed with the native 18 N-terminal amino acids of NT048 (MKSVPLITGGLSFLLSAC; SEQ ID NO: 88) or can be expressed with an alternative N-terminal sequence e.g. with a simple N-terminus methionine, or Met-Ala-, or a leader peptide which targets or traffics the expressed protein in a desired fashion.

NT053 Antigen

The antigen has been annotated as gi-145628236 in R2846 strain and cloned from said strain.

Useful NT053 antigens can elicit an antibody (e.g. when administered to a human) that recognises SEQ ID NO: 21 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 21; and/or (b) comprising a fragment of at least ‘n’ consecutive amino acids of SEQ ID NO: 21, wherein ‘n’ is 7 or more (e.g. 8, 10, 12, 14, 16, 18, 20, 25, 30, 35, 40, 50, 60, 70, 80, 90, 100, 150, 200, 250 or more). These NT053 proteins include variants of SEQ ID NO: 21. Preferred fragments of (b) comprise an epitope from SEQ ID NO: 21. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 21 while retaining at least one epitope of SEQ ID NO: 21. Other fragments omit one or more protein domains.

A NT053 antigen of the invention can be expressed with the native N-terminal Met of NT053 or can be expressed with an alternative N-terminal sequence e.g. with Met-Ala-, or a leader peptide which targets or traffics the expressed protein in a desired fashion.

NT066 Antigen

The antigen has been annotated as NTHI1230 in NP86-028 strain and localized in the periplasm of the bacteria. It has been cloned and expressed from Fi176 strain.

Useful NT066 antigens can elicit an antibody (e.g. when administered to a human) that recognises SEQ ID NO: 22 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 22; and/or (b) comprising a fragment of at least ‘n’ consecutive amino acids of SEQ ID NO: 22, wherein ‘n’ is 7 or more (e.g. 8, 10, 12, 14, 16, 18, 20, 25, 30, 35, 40, 50, 60, 70, 80, 90, 100, 150, 200, 250 or more). These NT066 proteins include variants of SEQ ID NO: 22, such as SEQ ID NO: 67. Preferred fragments of (b) comprise an epitope from SEQ ID NO: 22. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25, 27, 30, 33 or more) from the N-terminus of SEQ ID NO: 22 while retaining at least one epitope of SEQ ID NO: 22. Other fragments omit one or more protein domains.

A NT066 antigen of the invention can be expressed with the native 33 N-terminal amino acids of NT066 (MKIYLRFVWILIIILNFLLNLFITTNGVIIVNA; SEQ ID NO: 90) or can be expressed with an alternative N-terminal sequence e.g. with a simple N-terminus methionine, or Met-Ala-, or a leader peptide which targets or traffics the expressed protein in a desired fashion.

NT097 Antigen

The antigen has been annotated as NTHI0522 in NP86-028 strain and described as long-chain fatty acid FadL like transporter protein predicted to be present in the outer membrane milieu. It has been cloned and expressed from R2846 strain.

Useful NT097 antigens can elicit an antibody (e.g. when administered to a human) that recognises SEQ ID NO: 23 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 23; and/or (b) comprising a fragment of at least ‘n’ consecutive amino acids of SEQ ID NO: 23, wherein ‘n’ is 7 or more (e.g. 8, 10, 12, 14, 16, 18, 20, 25, 30, 35, 40, 50, 60, 70, 80, 90, 100, 150, 200, 250 or more). These NT097 proteins include variants of SEQ ID NO: 23. Preferred fragments of (b) comprise an epitope from SEQ ID NO: 23. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 22, 25 or more) from the N-terminus of SEQ ID NO: 23 while retaining at least one epitope of SEQ ID NO: 23. Other fragments omit one or more protein domains.

A NT097 antigen of the invention can be expressed with the native 22 N-terminal amino acids of NT097 (MKKFNQSILATAMLLAAGGANA; SEQ ID NO: 91) or can be expressed with an alternative N-terminal sequence e.g. with a simple N-terminus methionine, or Met-Ala-, or a leader peptide which targets or traffics the expressed protein in a desired fashion.

NT004 Antigen

The antigen has been annotated as hypothetical protein CGSHiGG_08215 from strain PittGG in the outer membrane milieu. It has been cloned and expressed from Fi 176 strain.

Useful NT004 antigens can elicit an antibody (e.g. when administered to a human) that recognises SEQ ID NO: 122 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 122; and/or (b) comprising a fragment of at least ‘n’ consecutive amino acids of SEQ ID NO: 122, wherein ‘n’ is 7 or more (e.g. 8, 10, 12, 14, 16, 18, 20, 25, 30, 35, 40, 50, 60, 70, 80, 90, 100, 150, 200, 250 or more). These NT004 proteins include variants of SEQ ID NO: 122. Preferred fragments of (b) comprise an epitope from SEQ ID NO: 122. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 22, 25 or more) from the N-terminus of SEQ ID NO: 122 while retaining at least one epitope of SEQ ID NO: 122. Other fragments omit one or more protein domains.

A NT004 antigen of the invention can be expressed with the native 20 N-terminal amino acids of NT004 (MKKKNQILVSLSIVALLGGC; SEQ ID NO: 125) or can be expressed with an alternative N-terminal sequence e.g. with a simple N-terminus methionine, or Met-Ala-, or a leader peptide which targets or traffics the expressed protein in a desired fashion.

NT014 Antigen

The antigen has been annotated as hypothetical protein HI1658 from strain Rd KW20. It has been cloned and expressed from Fi176 strain.

Useful NT014 antigens can elicit an antibody (e.g. when administered to a human) that recognises SEQ ID NO: 123 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 123; and/or (b) comprising a fragment of at least ‘n’ consecutive amino acids of SEQ ID NO: 123, wherein ‘n’ is 7 or more (e.g. 8, 10, 12, 14, 16, 18, 20, 25, 30, 35, 40, 50, 60, 70, 80, 90, 100, 150, 200, 250 or more). These NT014 proteins include variants of SEQ ID NO: 123. Preferred fragments of (b) comprise an epitope from SEQ ID NO: 123. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 22, 25 or more) from the N-terminus of SEQ ID NO: 123 while retaining at least one epitope of SEQ ID NO: 123. Other fragments omit one or more protein domains.

A NT014 antigen of the invention can be expressed with the native 22 N-terminal amino acids of NT014 (MTLSPLKKLAILLGATIFLQGC; SEQ ID NO: 126) or can be expressed with an alternative N-terminal sequence e.g. with a simple N-terminus methionine, or Met-Ala-, or a leader peptide which targets or traffics the expressed protein in a desired fashion.

NT022 Antigen

The antigen has been annotated as NTHI0830 from strain NP86-028 and identified to be a possible outer membrane antigenic lipoprotein B. It has been cloned and expressed from Fi176 strain. It has been also found to contain a LytM catalytic domain and to be surface exposed and secreted.

Useful NT022 antigens can elicit an antibody (e.g. when administered to a human) that recognises SEQ ID NO: 124 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 124; and/or (b) comprising a fragment of at least ‘n’ consecutive amino acids of SEQ ID NO: 124, wherein ‘n’ is 7 or more (e.g. 8, 10, 12, 14, 16, 18, 20, 25, 30, 35, 40, 50, 60, 70, 80, 90, 100, 150, 200, 250 or more). These NT022 proteins include variants of SEQ ID NO: 124. Preferred fragments of (b) comprise an epitope from SEQ ID NO: 124. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 22, 25 or more) from the N-terminus of SEQ ID NO: 124 while retaining at least one epitope of SEQ ID NO: 124. Other fragments omit one or more protein domains.

A NT022 antigen of the invention can be expressed with the native 18 N-terminal amino acids of NT022 (MKKSFLLLPLSLVVLSAC; SEQ ID NO: 127) or can be expressed with an alternative N-terminal sequence e.g. with a simple N-terminus methionine, or Met-Ala-, or a leader peptide which targets or traffics the expressed protein in a desired fashion.

Second Antigen Group

Antigen P48

The P48 polypeptide has been annotated in the literature as a Na(+)-translocating NADH-quinone reductase subunit A. For reference purposes, a full-length amino acid sequence of P48 is given as SEQ ID NO: 24 herein.

Preferred P48 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 24, e.g. 90% identity or more, or 95% identity or more, or 99% identity or more; and/or (b) comprising a fragment of at least ‘n’ consecutive amino acids of SEQ ID NO: 24, wherein ‘n’ is 7 or more (e.g. 8, 10, 12, 14, 16, 18, 20, 25, 30, 35, 40, 50, 60, 70, 80, 90, 100, 150, 200, 250 or more; e.g. 20 or more; or e.g. 50 or more; or e.g. 80 or more). These P48 polypeptides include variants of SEQ ID NO: 24. Preferred fragments of (b) comprise an epitope from SEQ ID NO: 24. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 24 while retaining at least one epitope of SEQ ID NO: 24. Other fragments omit one or more protein domains.

A P48 antigen of the invention ideally does not have the native 25 N-terminal amino acids of P48 (MITIKKGLDLPIAGKPAQVIHSGNA; SEQ ID NO: 92) and so it should be expressed with an alternative N-terminal sequence e.g. with a simple N-terminus methionine, or Met-Ala-, or a leader peptide which targets or traffics the expressed protein in a desired fashion.

According to the invention, the P48 antigen may advantageously be combined with one or more (e.g. 1, 2 or 3) of antigens HtrA, PE, P26, PHiD antigen and/or P6 as described herein, in particular, e.g. with HtrA.

Antigen HtrA

The HtrA polypeptide has been annotated in the literature as a periplasmic serine protease do/HhoA-like precursor, and has been described as a heat-shock protein or chaperone. For reference purposes, a full-length amino acid sequence of HtrA is given as SEQ ID NO: 25 herein.

Preferred HtrA polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 25, e.g. 90% identity or more, or 95% identity or more, or 99% identity or more; and/or (b) comprising a fragment of at least ‘n’ consecutive amino acids of SEQ ID NO: 25, wherein ‘n’ is 7 or more (e.g. 8, 10, 12, 14, 16, 18, 20, 25, 30, 35, 40, 50, 60, 70, 80, 90, 100, 150, 200, 250 or more; e.g. 20 or more; or e.g. 50 or more; or e.g. 80 or more). These HtrA polypeptides include variants of SEQ ID NO: 25. Preferred fragments of (b) comprise an epitope from SEQ ID NO: 25. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 25 while retaining at least one epitope of SEQ ID NO: 25. Other fragments omit one or more polypeptide domains.

A HtrA antigen of the invention ideally does not have the native 26 N-terminal amino acids of HtrA (MKKTRFVLNSIALGLSVLSTSFVAQA; SEQ ID NO: 93) and so it should be expressed with an alternative N-terminal sequence e.g. with a simple N-terminus methionine, or Met-Ala-, or a leader peptide which targets or traffics the expressed protein in a desired fashion.

According to the invention, the HtrA antigen may advantageously be combined with one or more (e.g. 1, 2, or 3) of antigens P48, PE, P26, P6 and/or PHiD, in particular, e.g. with P48.

Antigen PE

The PE polypeptide has been annotated as Lipoprotein-Vitronectin binding protein, or as binding IgD and acting as an adhesion to type 2 alveolar cells. For reference purposes, a full-length amino acid sequence of PE is given as SEQ ID NO: 26 herein.

Preferred PE polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 26, e.g. 90% identity or more, or 95% identity or more, or 99% identity or more; and/or (b) comprising a fragment of at least ‘n’ consecutive amino acids of SEQ ID NO: 26, wherein ‘n’ is 7 or more (e.g. 8, 10, 12, 14, 16, 18, 20, 25, 30, 35, 40, 50, 60, 70, 80, 90, 100, 150 or more; e.g. 20 or more; or e.g. 50 or more; or e.g. 80 or more). These PE polypeptides include variants of SEQ ID NO: 26. Preferred fragments of (b) comprise an epitope from SEQ ID NO: 26. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 26 while retaining at least one epitope of SEQ ID NO: 26. Other fragments omit one or more polypeptide domains.

A PE antigen of the invention ideally does not have the native 16 N-terminal amino acids of PE (MKKIILTLSLGLLTAC; SEQ ID NO: 94) and so it should be expressed with an alternative N-terminal sequence e.g. with a simple N-terminus methionine, or Met-Ala-, or a leader peptide which targets or traffics the expressed protein in a desired fashion.

According to the invention, the PE antigen may advantageously be combined with one or more (e.g. 1, 2 or 3) of antigens P48, HtrA, P26, P6 and/or PHiD as described herein.

Antigen P26

The P26 polypeptide is also known as outer membrane protein 26. It has been annotated as a member of the Skp family of proteins, whose putative function is translocation of outer membrane proteins [5]. For reference purposes, a full-length amino acid sequence of P26 is given as SEQ ID NO: 27 herein.

Preferred P26 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 27, e.g. 90% identity or more, or 95% identity or more, or 99% identity or more; and/or (b) comprising a fragment of at least ‘n’ consecutive amino acids of SEQ ID NO: 27, wherein ‘n’ is 7 or more (e.g. 8, 10, 12, 14, 16, 18, 20, 25, 30, 35, 40, 50, 60, 70, 80, 90, 100, 150 or more; e.g. 20 or more; or e.g. 50 or more; or e.g. 80 or more). These P26 polypeptides include variants of SEQ ID NO:27. Preferred fragments of (b) comprise an epitope from SEQ ID NO: 27. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 27 while retaining at least one epitope of SEQ ID NO: 27. Other fragments omit one or more protein domains.

According to the invention, the P26 antigen may advantageously be combined with one or more (e.g. 1, 2, or 3) of the antigens P48, HtrA, PE, PHiD and/or P6 as described herein, in particular with either or all of P48, HtrA and or PE as described herein.

PHiD Antigen

PHiD antigen is known also as “protein D” and has been used primarily as carrier protein in glycoconjugate NTHi vaccine approaches [95]. This antigen has been cloned and expressed from Fil76 strain.

Preferred PHiD polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 28, e.g. 90% identity or more, or 95% identity or more, or 99% identity or more; and/or (b) comprising a fragment of at least ‘n’ consecutive amino acids of SEQ ID NO: 28, wherein ‘n’ is 7 or more (e.g. 8, 10, 12, 14, 16, 18, 20, 25, 30, 35, 40, 50, 60, 70, 80, 90, 100, 150 or more; e.g. 20 or more; or e.g. 50 or more; or e.g. 80 or more). These PHiD polypeptides include variants of SEQ ID NO: 28. Preferred fragments of (b) comprise an epitope from SEQ ID NO: 28. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 28 while retaining at least one epitope of SEQ ID NO: 28. Other fragments omit one or more protein domains.

A PhiD antigen of the invention can be expressed with the native 18 N-terminal amino acids of PhiD (MKLKTLALSLLAAGVLAG; SEQ ID NO: 95) or can be expressed with an alternative N-terminal sequence e.g. with a simple N-terminus methionine, or Met-Ala-, or a leader peptide which targets or traffics the expressed protein in a desired fashion.

According to the invention, the PHiD antigen may advantageously combined with one or more (e.g. 1, 2, or 3) of any of the antigens P48, HtrA, PE, P26, and/or P6 as described herein.

A PhiD antigen of the invention can be a lipoprotein e.g. lipidated at a N-terminus cysteine.

P6 Antigen

P6 antigen is known also as OMP 6 (Outer membrane protein 6) [14]. This antigen was cloned and expressed from Fi176 strain.

Preferred P6 polypeptides for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 29, e.g. 90% identity or more, or 95% identity or more, or 99% identity or more; and/or (b) comprising a fragment of at least ‘n’ consecutive amino acids of SEQ ID NO: 29, wherein ‘n’ is 7 or more (e.g. 8, 10, 12, 14, 16, 18, 20, 25, 30, 35, 40, 50, 60, 70, 80, 90, 100, 150 or more; e.g. 20 or more; or e.g. 50 or more; or e.g. 80 or more). These P6 polypeptides include variants of SEQ ID NO: 29. Preferred fragments of (b) comprise an epitope from SEQ ID NO: 29. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 29 while retaining at least one epitope of SEQ ID NO: 29. Other fragments omit one or more protein domains.

A P6 antigen of the invention can be expressed with the native 19 N-terminal amino acids of P6 (MNKFVKSLLVAGSVAALAA; SEQ ID NO: 96) or can be expressed with an alternative N-terminal sequence e.g. with a simple N-terminus methionine, or Met-Ala-, or a leader peptide which targets or traffics the expressed protein in a desired fashion.

According to the invention, the P6 antigen may advantageously combined with one or more (e.g. 1, 2, or 3) of any of the antigens P48, HtrA, PE, P26, and/or PHiD as described herein.

A P6 antigen of the invention can be a lipoprotein e.g. lipidated at a N-terminus cysteine.

Third Antigen Group

NT013 Antigen

The “NT013” antigen is annotated as TPR repeat-containing protein and also as cytochrome c maturation heme lyase subunit CcmH2. It has been released as NTHI0532 in the strain 86-028NP. NT013 has been annotated as belonging to the metalloprotease protein family and it has a LytM catalytic domain.

Useful NT013 antigens can elicit an antibody (e.g. when administered to a human) that recognises SEQ ID NO: 30 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 30; and/or (b) comprising a fragment of at least ‘n’ consecutive amino acids of SEQ ID NO: 30, wherein ‘n’ is 7 or more (e.g. 8, 10, 12, 14, 16, 18, 20, 25, 30, 35, 40, 50, 60, 70, 80, 90, 100, 150, 200, 250 or more). These NT013 proteins include variants of SEQ ID NO: 30. Preferred fragments of (b) comprise an epitope from SEQ ID NO: 30. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 30 while retaining at least one epitope of SEQ ID NO: 30. Other fragments omit one or more protein domains.

A NT013 antigen of the invention can be expressed with the native 42 N-terminal amino acids of NT013 (MPVQHVKLARDRRKKRTYIKVGVFFVAILLILTGILLTIKDK; SEQ ID NO: 97) or can be expressed with an alternative N-terminal sequence e.g. with a simple N-terminus methionine, or Met-Ala-, or a leader peptide which targets or traffics the expressed protein in a desired fashion.

NT106 Antigen

The “NT106” antigen is annotated as lipoprotein and has been released as NTHI0363 in the genome 86-028NP.

Useful NT106 antigens can elicit an antibody (e.g. when administered to a human) that recognises SEQ ID NO: 31 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 31; and/or (b) comprising a fragment of at least ‘n’ consecutive amino acids of SEQ ID NO: 31, wherein ‘n’ is 7 or more (e.g. 8, 10, 12, 14, 16, 18, 20, 25, 30, 35, 40, 50, 60, 70, 80, 90, 100, 150, 200, 250 or more). These NT106 proteins include variants of SEQ ID NO: 31. Preferred fragments of (b) comprise an epitope from SEQ ID NO: 31. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 31 while retaining at least one epitope of SEQ ID NO: 31. Other fragments omit one or more protein domains.

A NT106 antigen of the invention can be expressed with the native 17 N-terminal amino acids of NT106 (MKKIILNLVTAIILAGC; SEQ ID NO: 98) or can be expressed with an alternative N-terminal sequence e.g. with a simple N-terminus methionine, or Met-Ala-, or a leader peptide which targets or traffics the expressed protein in a desired fashion.

A NT106 antigen of the invention can be a lipoprotein e.g. lipidated at a N-terminus cysteine.

NT107 Antigen

The “NT107” antigen is annotated as “Heme/hemopexin-binding protein B” and has been released as NTHI0370 in the genome 86-028NP.

Useful NT107 antigens can elicit an antibody (e.g. when administered to a human) that recognises SEQ ID NO: 32 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 32; and/or (b) comprising a fragment of at least ‘n’ consecutive amino acids of SEQ ID NO: 32, wherein ‘n’ is 7 or more (e.g. 8, 10, 12, 14, 16, 18, 20, 25, 30, 35, 40, 50, 60, 70, 80, 90, 100, 150, 200, 250 or more). These NT107 proteins include variants of SEQ ID NO: 32. Preferred fragments of (b) comprise an epitope from SEQ ID NO: 32. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 32 while retaining at least one epitope of SEQ ID NO: 32. Other fragments omit one or more protein domains.

A NT107 antigen of the invention can be expressed with the native 28 N-terminal amino acids of NT107 (MKMRPRYSVIASAVSLGFVLSKSVMALG; SEQ ID NO: 99) or can be expressed with an alternative N-terminal sequence e.g. with a simple N-terminus methionine, or Met-Ala-, or a leader peptide which targets or traffics the expressed protein in a desired fashion.

NT108 Antigen

The “NT108” antigen is annotated as murein transglycosylase A lipoprotein. In the strain 86-028NP has been annotated as NTHI0205.

Useful NT108 antigens can elicit an antibody (e.g. when administered to a human) that recognises SEQ ID NO: 33 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 33; and/or (b) comprising a fragment of at least ‘n’ consecutive amino acids of SEQ ID NO: 33, wherein ‘n’ is 7 or more (e.g. 8, 10, 12, 14, 16, 18, 20, 25, 30, 35, 40, 50, 60, 70, 80, 90, 100, 150, 200, 250 or more). These NT108 proteins include variants of SEQ ID NO: 33. Preferred fragments of (b) comprise an epitope from SEQ ID NO: 33. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 33 while retaining at least one epitope of SEQ ID NO: 33. Other fragments omit one or more protein domains.

A NT108 antigen of the invention can be expressed with the native 24 N-terminal amino acids of NT108 (MSVCKPFWFKTFSISIITALLVSC; SEQ ID NO: 100) or can be expressed with an alternative N-terminal sequence e.g. with a simple N-terminus methionine, or Met-Ala-, or a leader peptide which targets or traffics the expressed protein in a desired fashion.

A NT108 antigen of the invention can be a lipoprotein e.g. lipidated at a N-terminus cysteine.

NT109 Antigen

This antigen is annotated as nitrate/nitrite sensor protein NarQand has been identified as NTHI0374 in the strain 86-028NP and found to be conserved in the strains analysed.

Useful NT109 antigens can elicit an antibody (e.g. when administered to a human) that recognises SEQ ID NO: 34 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 34; and/or (b) comprising a fragment of at least ‘n’ consecutive amino acids of SEQ ID NO: 34, wherein ‘n’ is 7 or more (e.g. 8, 10, 12, 14, 16, 18, 20, 25, 30, 35, 40, 50, 60, 70, 80, 90, 100, 150, 200, 250 or more). These NT109 proteins include variants of SEQ ID NO: 34. Preferred fragments of (b) comprise an epitope from SEQ ID NO: 34. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 34 while retaining at least one epitope of SEQ ID NO: 34. Other fragments omit one or more protein domains.

A NT109 antigen of the invention can be expressed with the native 33 N-terminal amino acids of NT109 (MYTKGSVSTRIAKYLFIILIVAGVISSLSLAIM; SEQ ID NO: 101) or can be expressed with an alternative N-terminal sequence e.g. with a simple N-terminus methionine, or Met-Ala-, or a leader peptide which targets or traffics the expressed protein in a desired fashion.

NT110 Antigen

This antigen has been annotated as NTHI0579 in the genome 86-028NP and is known as putative haemolysis TlyC. IT has been found associated to the outer membrane.

Useful NT110 antigens can elicit an antibody (e.g. when administered to a human) that recognises SEQ ID NO: 35.

Preferred fragments of (b) comprise an epitope from SEQ ID NO: 35. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 35 while retaining at least one epitope of SEQ ID NO: 35. Other fragments omit one or more protein domains.

A NT110 antigen of the invention can be expressed with the native 30 N-terminal amino acids of NT110 (MIMELFHTILAIVALILSSAVVSSAEISLA; SEQ ID NO: 102) or can be expressed with an alternative N-terminal sequence e.g. with a simple N-terminus methionine, or Met-Ala-, or a leader peptide which targets or traffics the expressed protein in a desired fashion.

NT111 Antigen

This antigen has been annotated as NTHI0837 in the strain 86-028NP and described as putative lipoprotein.

Useful NT111 antigens can elicit an antibody (e.g. when administered to a human) that recognises SEQ ID NO: 36 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 36; and/or (b) comprising a fragment of at least ‘n’ consecutive amino acids of SEQ ID NO: 36, wherein ‘n’ is 7 or more (e.g. 8, 10, 12, 14, 16, 18, 20, 25, 30, 35, 40, 50, 60, 70, 80, 90, 100, 150, 200, 250 or more). These NT111 proteins include variants of SEQ ID NO: 36. Preferred fragments of (b) comprise an epitope from SEQ ID NO: 36. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 36 while retaining at least one epitope of SEQ ID NO: 36. Other fragments omit one or more protein domains.

A NT111 antigen of the invention can be expressed with the native 19 N-terminal amino acids of NT111 (MKKTLVAALISSVILLTGC; SEQ ID NO: 103) or can be expressed with an alternative N-terminal sequence e.g. with a simple N-terminus methionine, or Met-Ala-, or a leader peptide which targets or traffics the expressed protein in a desired fashion.

A NT110 antigen of the invention can be a lipoprotein e.g. lipidated at a N-terminus cysteine.

NT112 Antigen

This antigen has been annotated as NTHI10849 from NP86-028 strain. It is annotated as VacJ lipoprotein.

Useful NT112 antigens can elicit an antibody (e.g. when administered to a human) that recognises SEQ ID NO: 37 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 37; and/or (b) comprising a fragment of at least ‘n’ consecutive amino acids of SEQ ID NO: 37, wherein ‘n’ is 7 or more (e.g. 8, 10, 12, 14, 16, 18, 20, 25, 30, 35, 40, 50, 60, 70, 80, 90, 100, 150, 200, 250 or more). These NT112 proteins include variants of SEQ ID NO: 37. Preferred fragments of (b) comprise an epitope from SEQ ID NO: 37. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 37 while retaining at least one epitope of SEQ ID NO: 37. Other fragments omit one or more protein domains.

A NT112 antigen of the invention can be expressed with the native 19 N-terminal amino acids of NT112 (MKTKVILTALLSAIALTGC; SEQ ID NO: 104) or can be expressed with an alternative N-terminal sequence e.g. with a simple N-terminus methionine, or Met-Ala-, or a leader peptide which targets or traffics the expressed protein in a desired fashion.

A NT112 antigen of the invention can be a lipoprotein e.g. lipidated at a N-terminus cysteine.

NT113 Antigen

This antigen has been annotated as NTHI10921 in 86-026NP strain and as lipoprotein.

Useful NT113 antigens can elicit an antibody (e.g. when administered to a human) that recognises SEQ ID NO: 38 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 38; and/or (b) comprising a fragment of at least ‘n’ consecutive amino acids of SEQ ID NO: 38, wherein ‘n’ is 7 or more (e.g. 8, 10, 12, 14, 16, 18, 20, 25, 30, 35, 40, 50, 60, 70, 80, 90, 100, 150, 200, 250 or more). These NT113 proteins include variants of SEQ ID NO: 38. Preferred fragments of (b) comprise an epitope from SEQ ID NO: 38. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 38 while retaining at least one epitope of SEQ ID NO: 38. Other fragments omit one or more protein domains.

A NT113 antigen of the invention can be expressed with the native 16 N-terminal amino acids of NT113 (MKKYLLLALLPFLYAC; SEQ ID NO: 105) or can be expressed with an alternative N-terminal sequence e.g. with a simple N-terminus methionine, or Met-Ala-, or a leader peptide which targets or traffics the expressed protein in a desired fashion.

A NT113 antigen of the invention can be a lipoprotein e.g. lipidated at a N-terminus cysteine.

NT114 Antigen

This antigen has been annotated as soluble lytic murein transglycosylase protein and as NTHI0995 in 86-026NP strain.

Useful NT114 antigens can elicit an antibody (e.g. when administered to a human) that recognises SEQ ID NO: 39 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 39; and/or (b) comprising a fragment of at least ‘n’ consecutive amino acids of SEQ ID NO: 39, wherein ‘n’ is 7 or more (e.g. 8, 10, 12, 14, 16, 18, 20, 25, 30, 35, 40, 50, 60, 70, 80, 90, 100, 150, 200, 250 or more). These NT114 proteins include variants of SEQ ID NO: 39. Preferred fragments of (b) comprise an epitope from SEQ ID NO: 39. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 39 while retaining at least one epitope of SEQ ID NO: 39. Other fragments omit one or more protein domains.

A NT114 antigen of the invention can be expressed with the native 19 N-terminal amino acids of NT114 (MKKVALISLCIFTALSAFA; SEQ ID NO: 106) or can be expressed with an alternative N-terminal sequence e.g. with a simple N-terminus methionine, or Met-Ala-, or a leader peptide which targets or traffics the expressed protein in a desired fashion.

NT115 Antigen

This antigen has been annotated as NTHI1091 in 86-026NP strain and as putative LptE lipoprotein. It is located in the extracellular milieu.

Useful NT115 antigens can elicit an antibody (e.g. when administered to a human) that recognises SEQ ID NO: 40 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 40; and/or (b) comprising a fragment of at least ‘n’ consecutive amino acids of SEQ ID NO: 40, wherein ‘n’ is 7 or more (e.g. 8, 10, 12, 14, 16, 18, 20, 25, 30, 35, 40, 50, 60, 70, 80, 90, 100, 150, 200, 250 or more). These NT115 proteins include variants of SEQ ID NO: 40. Preferred fragments of (b) comprise an epitope from SEQ ID NO: 40. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 40 while retaining at least one epitope of SEQ ID NO: 40. Other fragments omit one or more protein domains.

A NT115 antigen of the invention can be expressed with the native 35 N-terminal amino acids of NT115 (MKYLHFTRPTIKVIFMINSIKTLLLIATLAILSAC; SEQ ID NO: 107) or can be expressed with an alternative N-terminal sequence e.g. with a simple N-terminus methionine, or Met-Ala-, or a leader peptide which targets or traffics the expressed protein in a desired fashion.

A NT115 antigen of the invention can be a lipoprotein e.g. lipidated at a N-terminus cysteine.

NT116 Antigen

This antigen has been described as NTHI1169 in 86-026NP strain and belongs to the transferrin-binding protein family.

Useful NT116 antigens can elicit an antibody (e.g. when administered to a human) that recognises SEQ ID NO: 41 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 41; and/or (b) comprising a fragment of at least ‘n’ consecutive amino acids of SEQ ID NO: 41, wherein ‘n’ is 7 or more (e.g. 8, 10, 12, 14, 16, 18, 20, 25, 30, 35, 40, 50, 60, 70, 80, 90, 100, 150, 200, 250 or more). These NT116 proteins include variants of SEQ ID NO: 41. Preferred fragments of (b) comprise an epitope from SEQ ID NO: 41. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 41 while retaining at least one epitope of SEQ ID NO: 41. Other fragments omit one or more protein domains.

A NT116 antigen of the invention can be expressed with the native 18 N-terminal amino acids of NT116 (MKSVPLITGGLSFLLSAC; SEQ ID NO: 108) or can be expressed with an alternative N-terminal sequence e.g. with a simple N-terminus methionine, or Met-Ala-, or a leader peptide which targets or traffics the expressed protein in a desired fashion.

NT117 Antigen

This antigen has been annotated as NTHI1208 in 86-026NP strain and putative transglutaminase. It has been located in the outer membrane.

Useful NT117 antigens can elicit an antibody (e.g. when administered to a human) that recognises SEQ ID NO: 42 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 42; and/or (b) comprising a fragment of at least ‘n’ consecutive amino acids of SEQ ID NO: 42, wherein ‘n’ is 7 or more (e.g. 8, 10, 12, 14, 16, 18, 20, 25, 30, 35, 40, 50, 60, 70, 80, 90, 100, 150, 200, 250 or more). These NT117 proteins include variants of SEQ ID NO: 42 Preferred fragments of (b) comprise an epitope from SEQ ID NO: 42 Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 42 while retaining at least one epitope of SEQ ID NO: 42. Other fragments omit one or more protein domains.

A NT117 antigen of the invention can be expressed with the native 19 N-terminal amino acids of NT117 (MKKLIAVAVFSACGSLAHA; SEQ ID NO: 109) or can be expressed with an alternative N-terminal sequence e.g. with a simple N-terminus methionine, or Met-Ala-, or a leader peptide which targets or traffics the expressed protein in a desired fashion.

NT118 Antigen

This antigen has been annotated as NTHI1318 in 86-026NP strain and as hypothetical protein.

Useful NT118 antigens can elicit an antibody (e.g. when administered to a human) that recognises SEQ ID NO: 43 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 43; and/or (b) comprising a fragment of at least ‘n’ consecutive amino acids of SEQ ID NO: 43, wherein ‘n’ is 7 or more (e.g. 8, 10, 12, 14, 16, 18, 20, 25, 30, 35, 40, 50, 60, 70, 80, 90, 100, 150, 200, 250 or more). These NT118 proteins include variants of SEQ ID NO: 43. Preferred fragments of (b) comprise an epitope from SEQ ID NO: 43. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 43 while retaining at least one epitope of SEQ ID NO: 43. Other fragments omit one or more protein domains.

A NT118 antigen of the invention can be expressed with the native 18 N-terminal amino acids of NT118 (MNIRWNVILGVIALCALA; SEQ ID NO: 110) or can be expressed with an alternative N-terminal sequence e.g. with a simple N-terminus methionine, or Met-Ala-, or a leader peptide which targets or traffics the expressed protein in a desired fashion.

NT119 Antigen

This antigen has been annotated as NTHI1565 hypothetical protein in 86-028NP strain.

Useful NT119 antigens can elicit an antibody (e.g. when administered to a human) that recognises SEQ ID NO: 114 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 114; and/or (b) comprising a fragment of at least ‘n’ consecutive amino acids of SEQ ID NO: 114, wherein ‘n’ is 7 or more (e.g. 8, 10, 12, 14, 16, 18, 20, 25, 30, 35, 40, 50, 60, 70, 80, 90, 100, 150, 200, 250 or more). These NT119 proteins include variants of SEQ ID NO: 114. Preferred fragments of (b) comprise an epitope from SEQ ID NO: 114. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 114 while retaining at least one epitope of SEQ ID NO: 114. Other fragments omit one or more protein domains.

A NT119 antigen of the invention can be expressed with the native 26 N-terminal amino acids of NT119 (MRFTKTLFTTALLGASIFSFQSTAWA; SEQ ID NO: 118) or can be expressed with an alternative N-terminal sequence e.g. with a simple N-terminus methionine, or Met-Ala-, or a leader peptide which targets or traffics the expressed protein in a desired fashion.

NT120 Antigen

This antigen has been annotated as NTHI1569 hypothetical protein in 86-028NP strain.

Useful NT120 antigens can elicit an antibody (e.g. when administered to a human) that recognises SEQ ID NO: 115 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 115; and/or (b) comprising a fragment of at least ‘n’ consecutive amino acids of SEQ ID NO: 115, wherein ‘n’ is 7 or more (e.g. 8, 10, 12, 14, 16, 18, 20, 25, 30, 35, 40, 50, 60, 70, 80, 90, 100, 150, 200, 250 or more). These NT120 proteins include variants of SEQ ID NO: 115. Preferred fragments of (b) comprise an epitope from SEQ ID NO: 115. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 115 while retaining at least one epitope of SEQ ID NO: 115. Other fragments omit one or more protein domains.

A NT120 antigen of the invention can be expressed with the native 26 N-terminal amino acids of NT120 (MKLTKTLLTTALFGASVFSFQSTAWA; SEQ ID NO: 119) or can be expressed with an alternative N-terminal sequence e.g. with a simple N-terminus methionine, or Met-Ala-, or a leader peptide which targets or traffics the expressed protein in a desired fashion.

NT121 Antigen

This antigen has been annotated as NTHI1571 hypothetical protein in 86-028NP strain.

Useful NT121 antigens can elicit an antibody (e.g. when administered to a human) that recognises SEQ ID NO: 116 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 116; and/or (b) comprising a fragment of at least ‘n’ consecutive amino acids of SEQ ID NO: 116, wherein ‘n’ is 7 or more (e.g. 8, 10, 12, 14, 16, 18, 20, 25, 30, 35, 40, 50, 60, 70, 80, 90, 100, 150, 200, 250 or more). These NT121 proteins include variants of SEQ ID NO: 116. Preferred fragments of (b) comprise an epitope from SEQ ID NO: 116. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 116 while retaining at least one epitope of SEQ ID NO: 116. Other fragments omit one or more protein domains.

A NT121 antigen of the invention can be expressed with the native 26 N-terminal amino acids of NT121 (MKLTKTLLTTALLGASVFSFQSTAWA; SEQ ID NO: 120) or can be expressed with an alternative N-terminal sequence e.g. with a simple N-terminus methionine, or Met-Ala-, or a leader peptide which targets or traffics the expressed protein in a desired fashion.

NT122 Antigen

This antigen has been annotated as NTHI1667 hypothetical protein in 86-028NP strain.

Useful NT122 antigens can elicit an antibody (e.g. when administered to a human) that recognises SEQ ID NO: 117 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 117; and/or (b) comprising a fragment of at least ‘n’ consecutive amino acids of SEQ ID NO: 117, wherein ‘n’ is 7 or more (e.g. 8, 10, 12, 14, 16, 18, 20, 25, 30, 35, 40, 50, 60, 70, 80, 90, 100, 150, 200, 250 or more). These NT122 proteins include variants of SEQ ID NO: 117. Preferred fragments of (b) comprise an epitope from SEQ ID NO: 117. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 117 while retaining at least one epitope of SEQ ID NO: 117. Other fragments omit one or more protein domains.

A NT122 antigen of the invention can be expressed with the native 23 N-terminal amino acids of NT122 (MEKIMKKLTLALVLGSALAVTGC; SEQ ID NO: 121) or can be expressed with an alternative N-terminal sequence e.g. with a simple N-terminus methionine, or Met-Ala-, or a leader peptide which targets or traffics the expressed protein in a desired fashion.

NT123 Antigen

This antigen has been annotated as zinc protease NTHI1796 in 86-026NP strain.

Useful NT123 antigens can elicit an antibody (e.g. when administered to a human) that recognises SEQ ID NO: 44 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 44; and/or (b) comprising a fragment of at least ‘n’ consecutive amino acids of SEQ ID NO: 44, wherein ‘n’ is 7 or more (e.g. 8, 10, 12, 14, 16, 18, 20, 25, 30, 35, 40, 50, 60, 70, 80, 90, 100, 150, 200, 250 or more). These NT123 proteins include variants of SEQ ID NO: 44. Preferred fragments of (b) comprise an epitope from SEQ ID NO: 44. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 44 while retaining at least one epitope of SEQ ID NO: 44. Other fragments omit one or more protein domains.

A NT123 antigen of the invention can be expressed with the native 17 N-terminal amino acids of NT123 (MKKTTALFLLIFSLIAC; SEQ ID NO: 111) or can be expressed with an alternative N-terminal sequence e.g. with a simple N-terminus methionine, or Met-Ala-, or a leader peptide which targets or traffics the expressed protein in a desired fashion.

NT124 Antigen

This antigen has been annotated as hypothetical protein NTHI1930 in 86-026NP strain.

Useful NT124 antigens can elicit an antibody (e.g. when administered to a human) that recognises SEQ ID NO: 45 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 45; and/or (b) comprising a fragment of at least ‘n’ consecutive amino acids of SEQ ID NO: 45, wherein ‘n’ is 7 or more (e.g. 8, 10, 12, 14, 16, 18, 20, 25, 30, 35, 40, 50, 60, 70, 80, 90, 100, 150, 200, 250 or more). These NT124 proteins include variants of SEQ ID NO: 45. Preferred fragments of (b) comprise an epitope from SEQ ID NO: 45. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 45 while retaining at least one epitope of SEQ ID NO: 45. Other fragments omit one or more protein domains.

A NT124 antigen of the invention can be expressed with the native 22 N-terminal amino acids of NT124 (MKKSKIAAGVVISLAAVWCAGA; SEQ ID NO: 89) or can be expressed with an alternative N-terminal sequence e.g. with a simple N-terminus methionine, or Met-Ala-, or a leader peptide which targets or traffics the expressed protein in a desired fashion.

NT061 Antigen

This antigen has been annotated as survival protein SurA-like protein NTHI0588 in 86-026NP strain.

Useful NT061 antigens antigens can elicit an antibody (e.g. when administered to a human) that recognises SEQ ID NO: 128 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 128; and/or (b) comprising a fragment of at least ‘n’ consecutive amino acids of SEQ ID NO: 128, wherein ‘n’ is 7 or more (e.g. 8, 10, 12, 14, 16, 18, 20, 25, 30, 35, 40, 50, 60, 70, 80, 90, 100, 150, 200, 250 or more). These NT061 proteins include variants of SEQ ID NO: 128. Preferred fragments of (b) comprise an epitope from SEQ ID NO: 128. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 128 while retaining at least one epitope of SEQ ID NO: 128. Other fragments omit one or more protein domains.

A NT061 antigen of the invention can be expressed with the native 27 N-terminal amino acids of NT061 (MKMKKFILKSFLLATLGCVAFTSMAQA; SEQ ID NO: 129) or can be expressed with an alternative N-terminal sequence e.g. with a simple N-terminus methionine, or Met-Ala-, or a leader peptide which targets or traffics the expressed protein in a desired fashion.

NT017 Antigen

This antigen has been annotated as survival protein SurA-like protein NTHI0915 in 86-026NP strain.

Useful NT017 antigens antigens can elicit an antibody (e.g. when administered to a human) that recognises SEQ ID NO: 130 and/or may comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 130; and/or (b) comprising a fragment of at least ‘n’ consecutive amino acids of SEQ ID NO: 130, wherein ‘n’ is 7 or more (e.g. 8, 10, 12, 14, 16, 18, 20, 25, 30, 35, 40, 50, 60, 70, 80, 90, 100, 150, 200, 250 or more). These NT017 proteins include variants of SEQ ID NO: 130. Preferred fragments of (b) comprise an epitope from SEQ ID NO: 130. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NO: 130 while retaining at least one epitope of SEQ ID NO: 130. Other fragments omit one or more protein domains.

A NT017 antigen of the invention can be expressed with the native 20 N-terminal amino acids of NT017 (MLRFGVNQKTSLLLTALLSC; SEQ ID NO: 131) or can be expressed with an alternative N-terminal sequence e.g. with a simple N-terminus methionine, or Met-Ala-, or a leader peptide which targets or traffics the expressed protein in a desired fashion.

Hybrid Polypeptides

The polypeptides used with the invention may be expressed individually or independently on separate polypeptide chains. Alternatively, two or more of the polypeptides used with the invention may also be expressed as a single polypeptide chain (a ‘hybrid’ polypeptide). Hybrid polypeptides can be represented by the formula NH₂-A-{X-L-}ₙ-B—COOH, wherein: A is an optional N-terminal amino acid sequence; B is an optional C-terminal amino acid sequence; n is an integer of 2 or more (e.g. 2, 3, 4, 5, 6, etc.); wherein at least one of the Xₙ is an amino acid sequence of an antigen of the invention (as described above); and L is an optional linker amino acid sequence. According to the invention, for example, each Xₙ may comprise the amino acid sequences of an antigen selected from the group consisting of: (1) NTHI0915 (NT018), (2) NTHI1416 (NT024), (3) NTHI2017 (NT032), (4) CGSHiGG_02400 (NT038, but this is not amongst preferred), (5) NTHI1292 (NT067), (6) NTHI0877 (NT001), (7) NTHI0266 (NT016), (8) CGSHiGG_00130 (NT052), (9) NTHI1627 (NT002), (10) NTHI1109 (NT026), (11) NTHI0821 (NT009), (12) NTHI0409 (NT025), (13) NTHI1954 (NT028), (14) NTHI0371 (NT029), (15) NTHI0509 (NT031), (16) NTHI0449 (NT015), (17) NTHI1473 (NT023), (18) gi-145633184 (NT100), (19) NTHI1110 (NT040), (20) gi-46129075 (NT048), (21) gi-145628236 (NT053), (22) NTHI1230 (NT066), (23) NTHI0522 (NT097), (24) P48 (NTHI0254 also defined as NT007), (25) HtrA (NTHI1905 also defined as NT006), (26) PE (NTHI0267 also defined as NT035), (27) P26 (NTHI0501 also defined as NT010), (28) PHiD (NTHI0811 also defined as NT080), (29) P6 (NTHI0501, also defined as NT081), (30) NT013, (31) NT106, (32) NT107, (33) NT108, (34) NT109, (35) NT110, (36) NT111, (37) NT112, (38) NT113, (39) NT114, (40) NT115, (41) NT116, (42) NT117, (43) NT118, (44) NT119, (45) NT120, (46) NT121, (47), NT122, (48) NT123, (49) NT124; (50) NT004; (51) NT014; (52) NT022 (also annotated as NTHI0830); (53) NT016 (also annotated as NTHI0266).

According to the invention, the Xₙ may comprise the amino acid sequences of two or more antigens selected from the group consisting of any of the antigen listed in the “First antigen group” and any of the antigen listed in the “Second antigen group”. Each Xₙ may be an amino acid sequence of an antigen of an antigen combination of the invention (as described above). In certain embodiments, n is 2. When n is 2, any combination of two of the antigens as described above may also be used in accordance with the invention. When n is 3, for example, any combination of the invention of three antigens as described above may be used. Generally, two or more of the Xₙ may be the same antigens or, when n is 2, 3, or 4, each Xₙ may be a different antigen. When two or more of the Xₙ are sequences of the same antigen), said two or more Xₙ may have the same polypeptide sequence or a different polypeptide sequence, e.g., may be different variants or fragments of the given antigen, as described above.

Where these antigens are defined in terms of (a) having 50% or more identity (e.g. 60%, 65%, 70%, 75%, 80%, 85%, 90%, 91%, 92%, 93%, 94%, 95%, 96%, 97%, 98%, 99%, 99.5% or more) to a given sequence; and/or (b) comprising a fragment of at least ‘n’ consecutive amino acids of a given sequence, wherein ‘n’ is 7 or more (e.g. 8, 10, 12, 14, 16, 18, 20, 25, 30, 35, 40, 50, 60, 70, 80, 90, 100, 150, 200, 250 or more), the level of identity in (a) and the value of ‘n’ in (b) may be the same for each X.

The leader peptide sequence in the wild-type form of each −X-moiety may be included or omitted in the hybrid protein. In some embodiments, the leader peptides will be deleted except for that of the −X-moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of {—X-L-}, linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG (SEQ ID NO:46) or GSGSGGGG (SEQ ID NO:47), with the Gly-Ser dipeptide being formed from a BamHI restriction site, thus aiding cloning and manipulation, and the (Gly)₄ tetrapeptide being a typical poly-glycine linker. Other suitable linkers, particularly for use as the final Lₙ are a Leu-Glu dipeptide.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. Hisₙ where n=3, 4, 5, 6, 7, 8, 9, 10 or more) such as SEQ ID NO: 48. Other suitable N-terminal amino acid sequences will be apparent to those skilled in the art. If X₁ lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue.

-B- is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. Hisₙ where n=3, 4, 5, 6, 7, 8, 9, 10 or more, such as SEQ ID NO: 68), or sequences which enhance protein stability. Other suitable C-terminal amino acid sequences will be apparent to those skilled in the art.

Strains and Variants

Antigens are defined above by reference to naming conventions from the literature e.g. the “NTHI” numbering (from the genome of strain 86-028NP) or CGSHiGG numbering (from the genome of strain PittGG). Such conventions are explained in more detail in reference 15 (particularly Table 1). Table V herein relates the existing nomenclature to the “NT” nomenclature used herein. Thus an exemplary amino acid and nucleotide sequence for any of the antigens of the invention can easily be found in public sequence databases for the indicated strains (together with additional information, such as functional annotations), but the invention is not limited to sequences from the 86-028NP, 3655 or PittGG strains. Genome sequences of several other NTHI strains are available (again, see Table 1 of reference 15). Standard search and alignment techniques can be used to identify in any of these (or other) further genome sequences the homolog of any particular sequence given herein. Moreover, the available sequences can be used to design primers for amplification of homologous sequences from other strains. Thus the invention is not limited to these specific strains, but rather encompasses such variants and homologs from other NTHI strains, as well as non-natural variants. In general, suitable variants of a particular SEQ ID NO include its allelic variants, its polymorphic forms, its homologs, its orthologs, its paralogs, its mutants, etc. For instance, SEQ ID Nos: 49, 52, 54, 55, 57-59, 64, 65 & 67 include mutations as described below.

Thus, for instance, polypeptides used with the invention may, compared to the SEQ ID NO herein, include one or more (e.g. 1, 2, 3, 4, 5, 6, 7, 8, 9, etc.) amino acid substitutions, such as conservative substitutions (i.e. substitutions of one amino acid with another which has a related side chain). Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. The polypeptides may also include one or more (e.g. 1, 2, 3, 4, 5, 6, 7, 8, 9, etc.) single amino acid deletions relative to the SEQ ID NO sequences. The polypeptides may also include one or more (e.g. 1, 2, 3, 4, 5, 6, 7, 8, 9, etc.) insertions (e.g. each of 1, 2, 3, 4 or 5 amino acids) relative to the SEQ ID NO sequences.

Similarly, a polypeptide used with the invention may comprise an amino acid sequence that:

(a) is identical (i.e. 100% identical) to a sequence disclosed in the sequence listing;

(b) shares sequence identity (e.g. 80%, 85%, 90%, 91%, 92%, 93%, 94%, 95%, 96%, 97%, 98%, 99%, 99.5% or more) with a sequence disclosed in the sequence listing;

(c) has 1, 2, 3, 4, 5, 6, 7, 8, 9 or 10 (or more) single amino acid alterations (deletions, insertions, substitutions), which may be at separate locations or may be contiguous, as compared to the sequences of (a) or (b); and/or

(d) when aligned with a particular sequence from the sequence listing using a pairwise alignment algorithm, each moving window of x amino acids from N-terminus to C-terminus (such that for an alignment that extends top amino acids, where p>x, there are p-x+1 such windows) has at least x·y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if x·y is not an integer then it is rounded up to the nearest integer. The preferred pairwise alignment algorithm is the Needleman-Wunsch global alignment algorithm [16], using default parameters (e.g. with Gap opening penalty=10.0, and with Gap extension penalty=0.5, using the EBLOSUM62 scoring matrix). This algorithm is conveniently implemented in the needle tool in the EMBOSS package [17].

Where hybrid polypeptides are used, the individual antigens within the hybrid (i.e. individual −X-moieties) may be from one or more strains. Where n=2, for instance, X₂ may be from the same strain as X₁ or from a different strain. Where n=3, the strains might be (i) X₁=X₂=X₃ (ii) X₁=X₂≠X₃ (iii) X₁≠X₂=X₃ (iv) X₁≠X₂≠X₃ or (v) X₁=X₃≠X₂, etc.

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus. N-terminus truncation can remove leader peptides e.g. to facilitate recombinant expression in a heterologous host. C-terminus truncation can remove anchor sequences e.g. to facilitate recombinant expression in a heterologous host.

In general, when an antigen comprises a sequence that is not identical to a NTHI sequence from the sequence listing (e.g. when it comprises a sequence listing with <100% sequence identity thereto, or when it comprises a fragment thereof) it is preferred in each individual instance that the antigen can elicit an antibody which recognises the respective NTHI sequence from the sequence listing.

Mutant Bacteria

The present invention, also provides a NTHi bacterium in which one or more of the antigens of the invention has/have been knocked out [18]. Techniques for producing knockout bacteria are well known, and knockout of genes from NTHi strains have been reported i.e. in Ref. 19. A knockout mutation may be situated in the coding region of the gene or may lie within its transcriptional control regions (e.g. within its promoter). A knockout mutation will reduce the level of mRNA encoding the antigen to <1% of that produced by the wild-type bacterium, preferably <0.5%, more preferably <0.1%, and most preferably to 0%.

The invention also provides a NTHI bacterium in which one or more of the antigens of the invention has a mutation which inhibits its activity. The gene encoding the antigen will have a mutation that changes the encoded amino acid sequence or abolishes its expression. Mutation may involve deletion, substitution, and/or insertion, any of which may be involve one or more amino acids.

One embodiment provides deletions of one or more genes codying for antigens of the invention.

It was known in E. coli that two components of the division machinery with LytM domains (EnvC and NlpD) are direct regulators of the cell wall hydrolases (amidases) responsible for cell separation (AmiA, AmiB and AmiC) [20]. It is also known that LytM metalloproteases in E. coli are absolutely required for daughter cell separation.

In one embodiment, the present invention provides NTHI genes codifying for polypeptides that have the LytM catalytic domain. Generally metalloproteases are identified as containing HxH and HxxxD aminoacid domains in their catalytic domains. Preferably, these one or more genes are codifying for any one of NT013, NT022 or NT017.

The present invention describes that the mutation or deletion of one or more genes encoding for polypeptides having in common the LytM catalytic domain results in a drastic change in the bacterial cell division and bacterial phenotype.

Inventors have also shown that said mutation or deletion results in the release of vesicles known as OMVs or outer membrane vesicles, whereas the same wild type NTHi strains do not normally release OMVs.

In one particularly preferred embodiment it is described that by deleting NT013 and/or NT022 not only the bacterial cell division is affected, but there is also a surprising production and release of outer membrane vesicles (OMVs) in NTHI strains, that normally do not release OMVs.

Preferred embodiments provide NTHI strains wherein the deletions of one or more genes codying for anyone of NT013 or NT022 or NT017. For instance, the genes deleted can be substituted with an antibiotic resistance cassette, such as the erytromycin resistance cassette. It has been found that all the above mentioned polypeptides have in common a LytM catalytic domain and are all metalloproteases.

It has been also found that the LytM domain in NT013 and NT022 is conserved. NT013 catalytic active site is represented by the following aminoacid motifs -HKGD- and -HLH- at the C-terminal portion. of NT022 catalytic active site is represented by the following aminoacid motifs -NKGID- and -KLH- at the C-terminal.

The invention also provides a bacterium, such as a NTHi bacterium, which hyper-expresses an antigen of the invention.

The invention also provides a bacterium, such as a NTHi bacterium, that constitutively expresses an antigen of the invention. The invention also provides a E. coli comprising at least a gene encoding an antigen of the invention, wherein the gene is under the control of an inducible promoter.

OMV Based Vaccine

Gram-negative bacteria are separated from the external medium by two successive layers of membrane structures. These structures, referred to as the cytoplasmic membrane and the outer membrane (OM), differ both structurally and functionally. The outer membrane plays an important role in the interaction of pathogenic bacteria with their respective hosts. Consequently, the surface exposed bacterial molecules represent important targets for the host immune response, making outer-membrane components attractive candidates in providing vaccine, diagnostic and therapeutics reagents.

Mutant bacteria of the invention are particularly useful for preparing bacterial outer membrane vesicles which include NTHi antigens (e.g. antigens of the invention), and which can be used as immunogens.

The invention also provides a bacterium, such as a NTHi bacterium, which hyper-expresses at least one antigen of the invention preferably by overproducing OMVs.

Up-regulation can be used to increase the levels of useful NTHi proteins in OMVs.

A method for producing a NTHi bacterium overproducing OMVs of the invention is also provided, which method comprises genetically modifying a Gram-negative bacterial strain by one or more of the following processes: (a) engineering the strain to downregulate expression of one or more Tol genes; and (b) mutating one or more gene(s) encoding a protein comprising a peptidoglycan-associated site to attenuate the peptidoglycan-binding activity of the protein(s); (c) by mutation or deletion of one or more genes encoding for polypeptides having in common the LytM catalytic domain. In one particularly preferred embodiment, the NTHi might not express active NT013, NT022 genes and/or any of Tol genes [19], [18].

The invention also provides a process for preparing a NTHi vesicle, comprising a step of treating a NTHi bacterium of the invention such that its outer membrane forms vesicles.

The invention also provides a process for preparing a NTHi vesicle, comprising a step of culturing a NTHi bacterium of the invention under conditions in which its outer membrane spontaneously sheds vesicles.

The invention also provides a NTHi bacterium which overproduces OMVs and which also hyperexpresses the antigens of the present invention.

Polypeptides Used with the Invention

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides used with the invention can be prepared by various means (e.g. recombinant expression, purification from cell culture, chemical synthesis, etc.). Recombinantly-expressed proteins are preferred, particularly for hybrid polypeptides.

Polypeptides used with the invention are preferably provided in purified or substantially purified form i.e. substantially free from other polypeptides (e.g. free from naturally-occurring polypeptides), particularly from other H. influenzae or host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the antigens in the compositions are separated from the whole organism with which the molecule is expressed.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labelling component. Also included are, for example, polypeptides containing one or more analogues of an amino acid (including, for example, unnatural amino acids, etc.), as well as other modifications known in the art. Polypeptides can occur as single chains or associated chains.

Polypeptides used with the invention may comprise a sequence —P-Q- or -Q-P—, wherein: —P— is an amino acid sequence as defined above and -Q- is not a sequence as defined above i.e. may be provided as fusion proteins. Where the N-terminus codon of —P— is not ATG, but this codon is not present at the N-terminus of a polypeptide, it will be translated as the standard amino acid for that codon rather than as a Met. Where this codon is at the N-terminus of a polypeptide, however, it will be translated as Met. Examples of -Q- moieties include, but are not limited to, histidine tags (i.e. Hisₙ where n=3, 4, 5, 6, 7, 8, 9, 10 or more e.g. SEQ ID NO: 68), maltose-binding protein, or glutathione-S-transferase (GST).

Polypeptides used with the invention may comprise sequence —P-Q- or -Q-P when initially expressed as a nascent protein, but in some embodiments the -Q- moiety may be absent from the protein at its point of use e.g. a leader peptide might be post-translationally cleaved.

A useful N-terminus sequence for expression is SEQ ID NO: 48.

Although expression of the polypeptides used with the invention may take place in a H. influenzae, the invention will usually use a heterologous host for expression (recombinant expression). The heterologous host may be prokaryotic (e.g. a bacterium) or eukaryotic. It may be E. coli, but other suitable hosts include Bacillus subtilis, Vibrio cholerae, Salmonella typhi, Salmonella typhimurium, Neisseria lactamica, Neisseria cinerea, Mycobacteria (e.g. M. tuberculosis), yeasts, etc. Compared to the wild-type H. influenzae genes encoding polypeptides of the invention, it is helpful to change codons to optimise expression efficiency in such hosts without affecting the encoded amino acids.

Polypeptides used with the invention may be synthesised by a process comprising a step of synthesising at least part of the polypeptide by chemical means.

Nucleic Acids

The invention also provides nucleic acids (e.g. combinations of nucleic acids, vectors, or vector combinations), encoding polypeptides used with the invention, combinations of polypeptides or hybrid polypeptides of the invention. It also provides nucleic acid comprising a nucleotide sequence that encodes one or more (e.g., 2, 3 or 4) polypeptides or hybrid polypeptides of the antigen combinations of the invention. A nucleic acid may be, e.g., a vector (e.g. a cloning or expression vector).

Nucleotide sequences encoding polypeptides of the one or more (at least one) antigen and antigen combinations of the invention are either known (see e.g. references 6-9) or may be designed according to the genetic code. Thus, in the context of the present invention, such a nucleotide sequence may encode one or more of: (1) NTHI0915 (NT018), (2) NTHI1416 (NT024), (3) NTHI2017 (NT032), (4) CGSHiGG_02400 (NT038), (5) NTHI1292 (NT067), (6) NTHI0877 (NT001), (7) NTHI0266 (NT016), (8) CGSHiGG_00130 (NT052), (9) NTHI1627 (NT002), (10) NTHI1109 (NT026), (11) NTHI0821 (NT009), (12) NTHI0409 (NT025), (13) NTHI1954 (NT028), (14) NTHI0371 (NT029), (15) NTHI0509 (NT031), (16) NTHI0449 (NT015), (17) NTHI1473 (NT023), (18) gi-145633184 (NT100), (19) NTHI1110 (NT040), (20) gi-46129075 (NT048), (21) gi-145628236 (NT053), (22) NTHI1230 (NT066), (23) NTHI0522 (NT097) or a P48 antigen (such as SEQ ID NO: 24); an HtrA antigen (such as SEQ ID NO: 25); a PE antigen (such as SEQ ID NO: 26); P26 antigen (such as SEQ ID NO: 27); a PHiD antigen (such as SEQ ID NO: 28); a P6 antigen (such as SEQ ID NO: 29), (24) NT004, (25) NT014, (26) NT022 or one or more antigens from the “third antigen group” or may encode an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more, e.g. 90% identity or more, or 95% identity or more, or 99% identity or more, to any of above mentioned polypeptides; and/or (b) comprising a fragment of at least ‘n’ consecutive amino acids of any of said polypeptides: 1, wherein ‘n’ is 7 or more (e.g. 8, 10, 12, 14, 16, 18, 20, 25, 30, 35, 40, 50, 60, 70, 80, 90, 100, 150, 200, 250 or more; e.g. 20 or more; or e.g. 50 or more; or e.g. 80 or more).

The invention also provides nucleic acid which can hybridize to these nucleic acids. Hybridization reactions can be performed under conditions of different “stringency”. Conditions that increase stringency of a hybridization reaction of widely known and published in the art (e.g. page 752 of reference 121). Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art (e.g. see refs, 21, 22 & 123).

A nucleic acid may hybridize to a target under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

The invention includes nucleic acid comprising sequences complementary to these sequences (e.g. for antisense or probing, or for use as primers).

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids encoding antigens described herein are preferably provided in purified or substantially purified form i.e. substantially free from other nucleic acids (e.g. free from naturally-occurring nucleic acids), particularly from other H. influenzae or host cell nucleic acids, generally being at least about 50% pure (by weight), and usually at least about 90% pure. Nucleic acids of the invention are preferably H. influenzae nucleic acids.

Nucleic acids encoding antigens described herein may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acids may be attached to a solid support (e.g. a bead, plate, filter, film, slide, microarray support, resin, etc.). Nucleic acids may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids encoding antigens described herein may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids encoding antigens described herein can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid encoding antigens described herein, wherein the nucleic acid is synthesised in part or in whole using chemical means.

The invention provides vectors comprising nucleotide sequences encoding antigens described herein (e.g. cloning or expression vectors) and host cells transformed with such vectors.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Immunogenic Compositions and Medicaments

Immunogenic compositions of the invention may be useful as vaccines. Vaccines according to the invention may either be prophylactic (i.e. to prevent infection) or therapeutic (i.e. to treat infection), but will typically be prophylactic.

Compositions may thus be pharmaceutically acceptable. They will usually include components in addition to the antigens e.g. they typically include one or more pharmaceutical carrier(s) and/or excipient(s). A thorough discussion of such components is available in reference 118.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To improve thermal stability, a composition may include a temperature protective agent. Further details of such agents are provided below.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention can also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:
mineral salts, such as aluminium salts and calcium salts, including hydroxides (e.g. oxyhydroxides), phosphates (e.g. hydroxyphosphates, orthophosphates) and sulphates, etc. [e.g. see chapters 8 & 9 of ref 23];
oil-in-water emulsions, such as squalene-water emulsions, including MF59 (5% Squalene, 0.5% Tween 80, and 0.5% Span 85, formulated into submicron particles using a microfluidizer) (Chapter 10 of ref. 23; see also refs. 24-26, and chapter 12 of ref 27], complete Freund's adjuvant (CFA) and incomplete Freund's adjuvant (IFA);
saponin formulations [chapter 22 of ref. 23], such as QS21 [28] and ISCOMs [chapter 23 of ref 23];
virosomes and virus-like particles (VLPs) [29-35];
bacterial or microbial derivatives, such as non-toxic derivatives of enterobacterial lipopolysaccharide (LPS), Lipid A derivatives [36, 37], immunostimulatory oligonucleotides [38-43], such as IC-31™ [44] (deoxynucleotide comprising 26-mer sequence 5′-(IC)₁₃-3′ (SEQ ID NO: 112) and polycationic polymer peptide comprising 11-mer amino acid sequence KLKLLLLLKLK (SEQ ID NO: 113) and ADP-ribosylating toxins and detoxified derivatives thereof [45-54];
human immunomodulators, including cytokines, such as interleukins (e.g. IL-1, IL-2, IL-4, IL-5, IL-6, IL-7, IL-12 [55, 56], interferons (e.g. interferon-γ), macrophage colony stimulating factor, and tumor necrosis factor;
bioadhesives and mucoadhesives, such as chitosan and derivatives thereof, esterified hyaluronic acid microspheres [57] or mucoadhesives, such as cross-linked derivatives of poly(acrylic acid), polyvinyl alcohol, polyvinyl pyrollidone, polysaccharides and carboxymethylcellulose [58];
microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.);
liposomes [Chapters 13 & 14 of ref. 23, ref. 59-61];
polyoxyethylene ethers and polyoxyethylene esters [62];
—PCPP formulations [63 and 64];
muramyl peptides, including N-acetyl-muramyl-L-threonyl-D-isoglutamine (thr-MDP), N-acetyl-normuramyl-l-alanyl-d-isoglutamine (nor-MDP), and N-acetylmuramyl-l-alanyl-d-isoglutaminyl-l-alanine-2-(1′-2′-dipalmitoyl-sn-glycero-3-hydroxyphosphoryloxy)-ethylamine MTP-PE); and
imidazoquinolone compounds, including Imiquamod and its homologues (e.g. “Resiquimod 3M”) [65 and 66].

Immunogenic compositions and vaccines of the invention may also comprise combinations of aspects of one or more of the adjuvants identified above. For example, the following adjuvant compositions may be used in the invention: (1) a saponin and an oil-in-water emulsion [67]; (2) a saponin (e.g. QS21)+a non-toxic LPS derivative (e.g. 3dMPL) [68]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [69]; (5) combinations of 3dMPL with, for example, QS21 and/or oil-in-water emulsions [70]; (6) SAF, containing 10% squalne, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3 dMPL).

Other substances that act as immunostimulating agents are disclosed in chapter 7 of ref 23.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum. A combination of aluminium phosphate and 3dMPL may be used (this has been reported as effective in pneumococcal immunisation [71]). The use of an MF59 adjuvant is preferred, in particular in case of IM (intramuscular) or IP (Intraperitoneal) immunization.

The compositions of the invention may elicit both a cell mediated immune response as well as a humoral immune response. This immune response will preferably induce long lasting (e.g. neutralising) antibodies and a cell mediated immunity that can quickly respond upon exposure to NTHI.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MEC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA. H. influenzae infections can affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Where more than one antigen is included in a composition then two antigens may be present at the same dose as each other or at different doses.

As mentioned above, a composition may include a temperature protective agent, and this component may be particularly useful in adjuvanted compositions (particularly those containing a mineral adjuvant, such as an aluminium salt). As described in reference 72, a liquid temperature protective agent may be added to an aqueous vaccine composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGS may have an average molecular weight ranging from 200-20,000 Da. In a preferred embodiment, the polyethylene glycol can have an average molecular weight of about 300 Da (PEG-300′).

Compositions of the invention may be formed by mixing (i) an aqueous composition comprising two or more (e.g. 1, 2, 3, 4) antigen(s) of the antigen combinations of the invention with (ii) a temperature protective agent. The mixture may then be stored e.g. below 0° C., from 0-20° C., from 20-35° C., from 35-55° C., or higher. It may be stored in liquid or frozen form. The mixture may be lyophilised. The composition may alternatively be formed by mixing (i) a dried composition comprising two or more (e.g. 1, 2, 3, 4) antigen(s) of the antigen combinations of the invention, with (ii) a liquid composition comprising the temperature protective agent. Thus component (ii) can be used to reconstitute component (i).

Methods of Treatment, and Administration of the Vaccine

The invention also provides a method for raising an immune response in a mammal comprising the step of administering an effective amount of a composition of the invention, or one or more steps of administering at least one or more (e.g. 1, 2, 3, 4, 5, 6, 7, 8, 9 or 10) antigens of the invention. The immune response is preferably protective and preferably involves antibodies and/or cell-mediated immunity. The method may raise a booster response.

The invention also provides at least one or more antigens of the invention for combined use as a medicament e.g. for use in raising an immune response in a mammal.

The invention also provides the use of at least one or more antigens of the invention in the manufacture of a medicament for raising an immune response in a mammal.

In the methods and uses of the invention, at least one or more (e.g. 1, 2, 3, 4) antigens of the invention may be administered simultaneously, separately or sequentially.

By raising an immune response in the mammal by these uses and methods, the mammal can be protected against H. influenzae infection. The invention is effective against H. influenzae of various different serotypes, but can be particularly useful in protecting against disease resulting from infection by non-typeable H. influenzae (NTHI). In accordance with the invention, an infection may be associated with a disease or condition selected from, for instance, otitis media (including acute otitis media), bronchitis, conjunctivitis, sinusitis, a urinary tract infection, pneumonia, bacteremia, septic arthritis, epiglottitis, pneumonia, empyema, pericarditis, cellulitis, osteomyelitis, lower respiratory tract infection or meningitis. The invention is particularly useful for treating or preventing inflammation of the middle ear or for treating or preventing COPD diseases, by eliciting an immune response that prevents bacteria from moving from the throat to the middle ear via the eustachian tube, where the middle ear is then colonised.

The invention also provides a kit comprising a first component and a second component wherein neither the first component nor the second component is a composition of the invention as described above, but wherein the first component and the second component can be combined to provide a composition of the invention as described above. The kit may further include a third component comprising one or more of the following: instructions, syringe or other delivery device, adjuvant, or pharmaceutically acceptable formulating solution.

The invention also provides a delivery device pre-filled with an immunogenic composition of the invention.

The mammal is preferably a human, e.g. human patient. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc. A mammal (e.g. human, e.g. a patient) may either be at risk from the disease themselves or may be a pregnant female, e.g. woman (‘maternal immunisation’).

One way of checking efficacy of therapeutic treatment involves monitoring H. influenzae infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Immunogenicity of compositions of the invention can be determined by administering them to test subjects (e.g. children 12-16 months age, or animal models such as a chinchilla model [73]) and then determining standard parameters including ELISA titres (GMT) of IgG. These immune responses will generally be determined around 4 weeks after administration of the composition, and compared to values determined before administration of the composition. Where more than one dose of the composition is administered, more than one post-administration determination may be made. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of vaccine compositions can also be determined in vivo by immunization studies in animal models of H. influenzae infection, e.g., guinea pigs Chinchillas, or mice, with the vaccine compositions. One such model is described in reference 74.

Other useful animal model to be used to determine in vivo the efficacy of vaccine compositions of the invention is described in reference 75.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosal, such as by rectal, oral, (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. >50 years old, >60 years old, and preferably >65 years), the young (e.g. <5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc.

Mucosal Immunisation

The invention provides the antigens, antigen combinations, and compositions of the invention for mucosal immunisation. E.g., the invention provides an immunogenic composition comprising (i) a polypeptide antigen combination of the invention, and (ii) a bacterial ADP-ribosylating toxin and or detoxified derivative thereof. The invention also provides a method for raising an immune response in a mammal comprising the step of administering an effective amount of such an immunogenic composition to the mammal. The composition is preferably administered via mucosa (to a mucosal surface) e.g. it may be administered intranasal.

The toxin of component (ii) may be, for example, derived from E. coli heat labile enterotoxin (“LT”). The derivative may have a detoxifying mutation in its A subunit e.g. it may be LT-K63 or LT-R72. In particular it may be LT-K63. In other embodiments, it is not LT-K63.

Intranasal administration of antigens or compositions of the invention and a LT-K63 adjuvant is preferred. This may decrease the H. influenzae bacterial load in the nasopharynx, lungs and blood, and increase survival rate of infected mammals.

Further antigenic components of compositions of the invention

The invention also provides compositions further comprising at least one further non-typeable H. influenzae antigen.

The invention also provides compositions further comprising at least one antigen that is not a non-typeable H. influenzae antigen.

In particular, the invention also provides a composition comprising one or more polypeptides of the invention and one or more of the following further antigens:
an antigen from N. meningitidis serogroup A, B, C, W135 and/or Y.
a saccharide or polypeptide antigen from Streptococcus pneumoniae [e.g. 76, 77, 78].
an antigen from hepatitis A virus, such as inactivated virus [e.g. 79, 80].
an antigen from hepatitis B virus, such as the surface and/or core antigens [e.g. 80, 81].
a diphtheria antigen, such as a diphtheria toxoid [e.g. chapter 3 of ref 82] or the CRM₁₉₇ mutant [e.g. 83].
a tetanus antigen, such as a tetanus toxoid [e.g. chapter 4 of ref. 82].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 84 & 85].
a whole cellular pertussis antigen
a saccharide antigen from Haemophilus influenzae B [e.g. 86].
polio antigen(s) [e.g. 87, 88] such as IPV.
measles, mumps and/or rubella antigens [e.g. chapters 9, 10 & 11 of ref 82].
influenza antigen(s) [e.g. chapter 19 of ref. 82], such as the haemagglutinin and/or neuraminidase surface proteins.
an antigen from Moraxella catarrhalis [e.g. 89].
an protein antigen from Streptococcus agalactiae (group B streptococcus) [e.g. 90, 91].
a saccharide antigen from Streptococcus agalactiae (group B streptococcus).
an antigen from Streptococcus pyogenes (group A streptococcus) [e.g. 91, 92, 93].
an antigen from Staphylococcus aureus [e.g. 94].
an antigen from Respiratory Syncytial Virus, e.g. a recombinant protein F [e.g. 142]
a vaccine composition comprising diphtheria (D), tetanus (T), pertussis (acellular, component) (Pa), hepatitis B (rDNA) (HBV), poliomyelitis (inactivated) (IPV) and Haemophilus influenzae type b (Hib) conjugate vaccine (adsorbed), e.g. Infanrix-hexa

The composition may comprise one or more of these further antigens. Combinations with a RSV vaccine and/or with a DTPa-containing vaccine are of particular interest.

Toxic protein antigens may be detoxified where necessary (e.g. detoxification of pertussis toxin by chemical and/or genetic means [85]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates include diphtheria toxin, tetanus toxin, the N. meningitidis outer membrane protein [95], synthetic peptides [96.97], heat shock proteins [98.99], pertussis proteins [100,101], protein D from H. influenzae [102], cytokines [103], lymphokines [103], streptococcal proteins, hormones [103], growth factors [103], toxin A or B from C. difficile [104], iron-uptake proteins [105], etc. A preferred carrier protein is the CRM197 mutant of diphtheria toxin [106].

Antigens in the composition will typically be present at a concentration of at least 1 μg/ml each. In general, the concentration of any given antigen will be sufficient to elicit an immune response against that antigen.

As an alternative to using proteins antigens in the immunogenic compositions of the invention, nucleic acid (preferably DNA e.g. in the form of a plasmid) encoding the antigen may be used.

Antigens are preferably adsorbed to an aluminium salt.

Antibodies

Antibodies against antigens according to the invention can be used for passive immunisation [107]. Thus the invention provides antibodies specific to antigens of the invention for use in therapy. These antibodies may be used singly or in combination. The invention also provides and immunogenic and pharmaceutical compositions comprising such antibodies.

The antibodies can be used in medicine and in therapy e.g. for passive immunisation against NTHI, or for clearing a NTHI infection. The invention also provides the use of such antibodies in the manufacture of a medicament. The invention also provides a method for treating a mammal comprising the step of administering an effective amount of an antibody of the invention. As described above for immunogenic compositions, these methods and uses allow a mammal to be protected against NTHI infections. In particular, antibodies of the invention may be used in methods of treating or preventing infections by NTHI, comprising the step of administering to the mammal an effective amount of an antibody as described herein, or a composition comprising such an antibody.

The term “antibody” includes intact immunoglobulin molecules (like palivizumab), as well as fragments thereof which are capable of binding a NTHI antigen. These include hybrid (chimeric) antibody molecules [108, 109]; F(ab′)2 and F(ab) fragments and Fv molecules; non-covalent heterodimers [110, 111]; single-chain Fv molecules (sFv) [112]; dimeric and trimeric antibody fragment constructs; minibodies [113, 114]; humanized antibody molecules [115-117]; and any functional fragments obtained from such molecules, as well as antibodies obtained through non-conventional processes such as phage display. Preferably, the antibodies are monoclonal antibodies. Methods of obtaining monoclonal antibodies are well known in the art. Humanised or fully-human antibodies are preferred. Antibodies and antibody combinations of the invention may be purified or isolated.

General

The practice of the present invention will employ, unless otherwise indicated, conventional methods of chemistry, biochemistry, molecular biology, immunology and pharmacology, within the skill of the art. Such techniques are explained fully in the literature. See, e.g., references 118-125, etc.

Where the invention concerns an “epitope”, this epitope may be a B-cell epitope and/or a T-cell epitope. Such epitopes can be identified empirically (e.g. using PEPSCAN [126,127] or similar methods), or they can be predicted (e.g. using the Jameson-Wolf antigenic index [128], matrix-based approaches [129], MAPITOPE [130], TEPITOPE [131,132], neural networks [133], OptiMer & EpiMer [134, 135], ADEPT [136], Tsites [137], hydrophilicity [138], antigenic index [139] or the methods disclosed in references 140-144, etc.). Epitopes are the parts of an antigen that are recognised by and bind to the antigen binding sites of antibodies or T-cell receptors, and they may also be referred to as “antigenic determinants”.

Where an antigen “domain” is omitted, this may involve omission of a signal peptide, of a cytoplasmic domain, of a transmembrane domain, of an extracellular domain, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref 22. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref 145.

### BRIEF DESCRIPTION OF DRAWINGS

FIGS. 1A, 1B and 1C show a mini-induction confirming strong expression of the antigens in BL21 (DE3)T1^{r} cells. (a): (LMWM: molecular weight standard markers)

FIG. 2 shows various results for NT001, NT007, NT018, NT024, NT032 and NT067. Similar expression results were obtained with the other preferred antigens, such as NT052, NT004, NT014, NT016 or NT022. Each panel shows western blot and FACS data. The western blots were performed using mouse sera, and lanes show reactivity with total bacterial extracts (“TE”), with vesicles prepared from NTHI outer membranes (“OMV”), or with purified recombination protein (“PP”). The FACS analyses follow incubation of inactivated bacteria with sera from mice immunized with various antigen compositions using only alum as negative control; pre-immune serum negative controls are shown as solid areas, and surface expression signal obtained with sample serum is shown as a single line.

FIG. 3 shows the layout on a 96 well plate of a serum bactericidal assay to verify the capacity of antisera against antigens of the invention to kill NTHI.

### MODES FOR CARRYING OUT THE INVENTION

Overview

Antigens list all of them which were identified as conserved in a comparative analysis performed by the inventors of at least 86 different NTHI strains, were cloned and expressed. The proteins were purified and used to immunize mice. Antisera from the immunized mice were used to verify surface localization and protective capability of the proteins used in immunization (Table III and/or Table IV). The results show that immunization NT052, NT024, NT032, NT001, NT067, NT004, NT014, NT022, NT016 is highly protective against NTHI and they showed higher or at least comparable bacterial killing activity SBA (Serum bactericidal assay) titers even compared with the “second antigen group”.

Strains and Variants

Inventors found that genes encoding NT022, NT016, NT014, NT018, NT024, NT032, NT067 and NT001 were present and conserved in all 86 genome sequences analysed.

The encoded NT018 sequences were 95-100% identical across the panel composed by the 15 complete genomes and the 32 strains from the Finnish otitis collection. The encoded NT024 sequences were 90-100% identical in the panel composed by the 15 complete genomes and the 32 strains from the Finnish otitis collection.

The encoded NT032 sequences were 95-100% identical in the panel composed by the 15 complete genomes and the 32 strains from the Finnish otitis collection; the encoded NT067 sequences were 95-100% identical in the panel composed by the 15 complete genomes and the 32 strains from the Finnish otitis collection. The encoded NT001 sequences were 95-100% identical in the panel composed by the 15 complete genomes and the 32 strains from the Finnish otitis collection.

Conservation in the encoded amino acid sequences are shown in Table I.

TABLE I
antigen conservation (% identity) amongst
Haemophilus genomes and strains

| Antigen | NT018 | NT024 | NT032 | NT067 | NT001 |
|---|---|---|---|---|---|
| % | 95-100 | 90-100 | 95-100 | 95-100 | 95-100 |

For expression purposes, antigens belonging to the “first antigen group” and/or “second antigen group” were cloned from either strain Fi176 which is one strain isolated from a Finnish collection of strains obtained from patients with otitis media or from strain R2846 [146]. Most of the antigen selected and further tested in animal model are also found to be well conserved amongst strains, e.g. NT016, NT067, NT022, NT014.

In some cases mutations have been introduced into the wild-type sequences. These mutations are underlined in the sequence listing for NT018, NT067, NT001, NT016, NT002, NT026, NT009, NT015, NT023 and NT066 (see SEQ ID NOs: 49, 52, 54, 55, 57-59, 64, 65 & 67).

Cloning and Expression of NTHI Recombinant Proteins

Cloning and expression of antigens can be performed by standard methods [121].

ORFs for antigens from NTHI strain Fi176 or R2846 were PCR-amplified using specific oligonucleotides and NTHI chromosomal DNA as template. Resulting PCR products were cloned in pET15b (Novagen) using the PIPE method [147], consisting in the PCR amplification of the cloning vector (V-PCR) and in the PCR amplification of the insert (I-PCR). Then, 1 μl of V-PCR and 1 μl of I-PCR are mixed and transformed in chemically competent HK100 cells [148]. I-PCR reactions were set up containing 1 μM each of the forward and reverse primers, lx Cloned Pfu DNA Polymerase Reaction Buffer, 2.5 units of Pfu Turbo DNA polymerase (Stratagene), 200 μM of each dNTP (Invitrogen) and 50 ng of genomic DNA template. The reactions were conducted as follows: initial denaturation for 2 min at 95° C., then 25 cycles of 95° C. for 30 s, 55° C. for 45 s, and 68° C. for 3 min followed by a final cool down to 4° C. V-PCR reactions were identical to the I-PCR reactions but the steps at 68° C. were lasting 14 min and 2 ng of pET15b plasmid were used as DNA template. Correct transformants where selected by PCR screening and DNA plasmid sequencing of the vector-insert junctions. The correct plasmid were then prepared from selected HK100 clones and used to transform BL21(DE3)T1^{r} cells (Sigma) in order to allow protein expression.

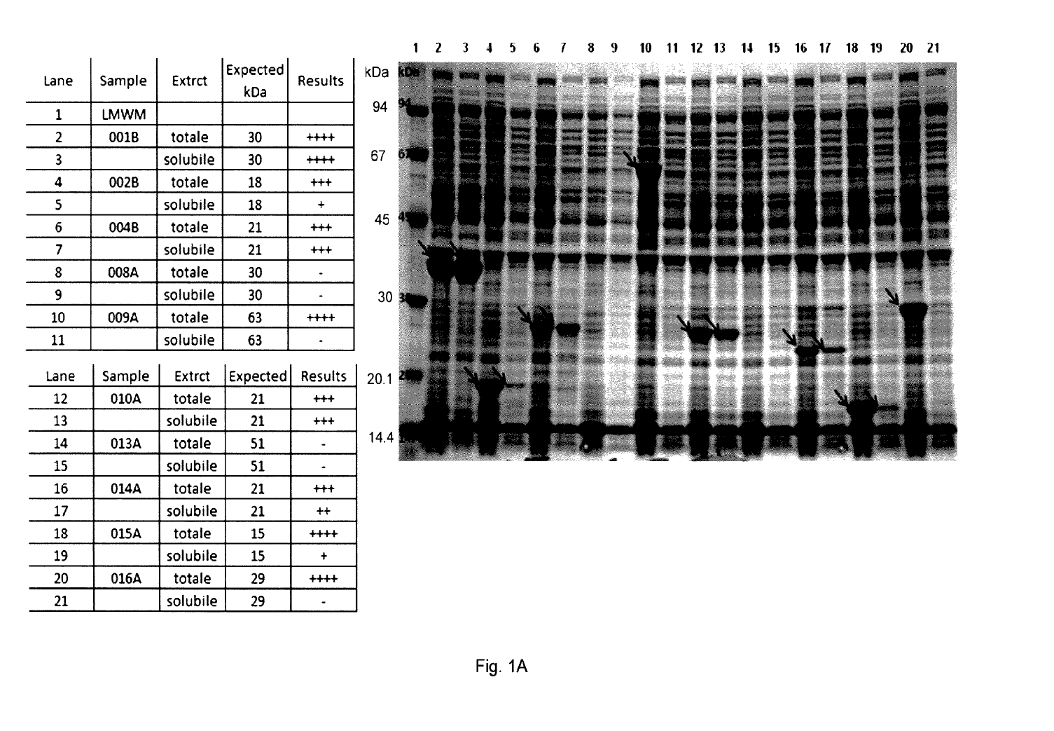

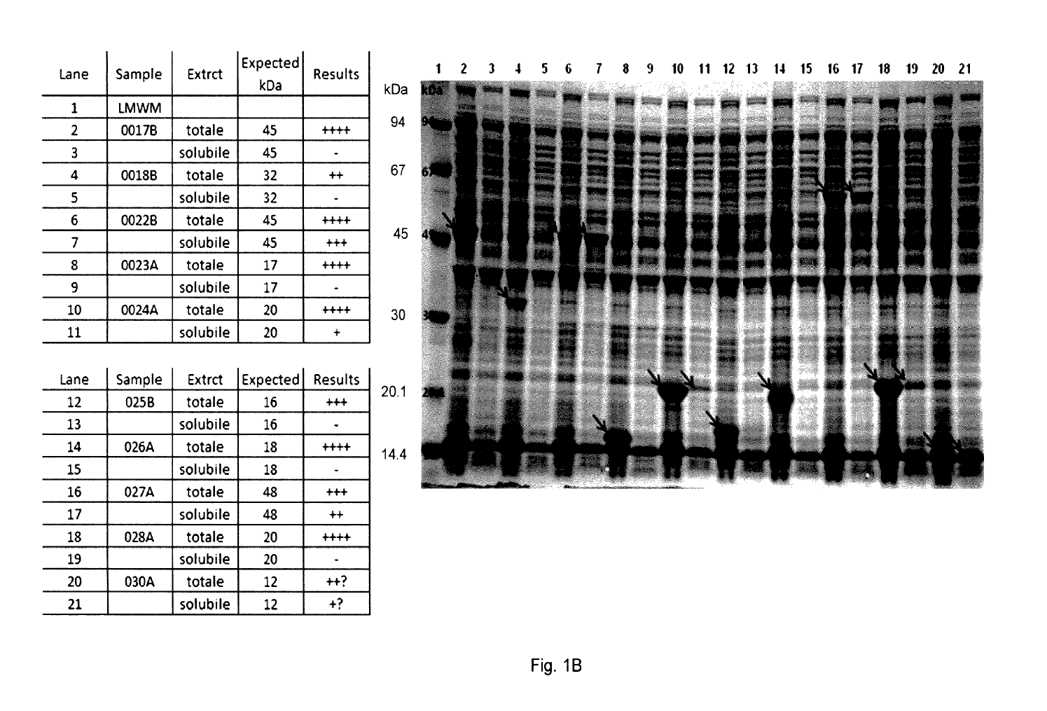

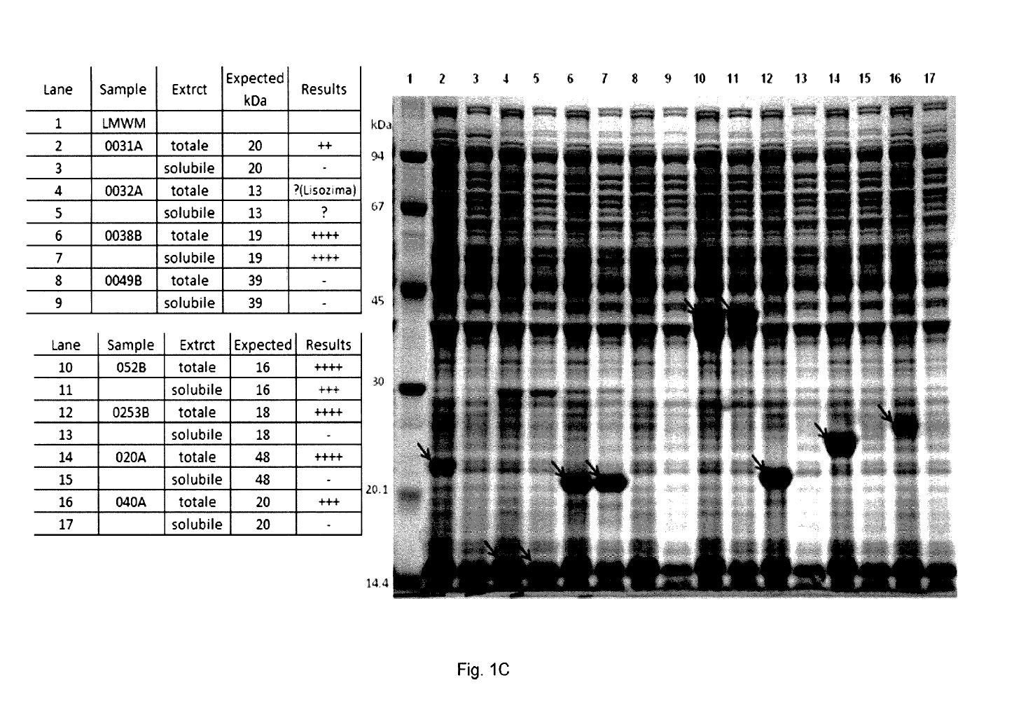

To express cloned proteins, BL21(DE3)T1^{r} clones containing pET15b constructs were grown in LB medium containing 100 μg/ml Ampicilin at 37° C. until OD₆₀₀=0.5. Protein expression was then induced by adding 1 mM IPTG and growing at the same temperature for additional 3 hrs. Conventional protein extractions and SDS-Page were performed to check protein expression. FIGS. 1A, 1B and 1C show a mini-induction confirming good expression of the antigens.

Protein Purification

Proteins were purified by the following general procedure: BL21(DE3)T1 wet biomass is suspended in lysis buffer and clarified by centrifugation. For purification of soluble protein ( ) supernatants after lysis are applied on His Multitrap HP 50 μl NiSepharose High Performance 96 well plates. For insoluble protein (HtrA, PE and P48), pellets containing the unsoluble fraction after lysis are solubilised with 6M Guanidine-HCl and re-centrifuged, and the supernatants applied to His Multitrap HP 50 ml NiSepharose High Performance 96 well plates.

Flow-through is collected and all wells washed with buffer containing 20 mM imidazole. His fusion proteins are then eluted with 250 mM imidazole. The procedure is performed using a vacuum system. Purified antigens are used in the immunisation schemes described herein.

The following protocol was followed:
1) Resuspend BL21(DE3)T1 pellet (1 g) in 1.5 ml B-PER™ (PIERCE) buffer, add 15 μl of lysozyme, 7.5 μl DNAse and 3 μl of MgCl₂ 1M
2) Incubate for 30 min for lysis
3) Centrifuge at 20000 rpm at 4° C. for 30 minute; for purification of any insoluble protein, solubilise pellets containing the unsoluble fraction after lysis with 6M Guanidine-HCl and re-centrifuge
4) Recover supernatant and filter (pore of 0.8 μm).
5) Use His Multitrap HP 50 μl NiSepharose High Performance 96 wells, connected to a vacuum system

Buffer A: 50 mM NaPPi, 300 mM NaCl, pH8

Buffer B: 50 mM NaPPi, 300 mM NaCl, 250 mM Imidazole, pH8

Buffer C: 50 mM NaPPi, 300 mM NaCl, 20 mM Imidazole, pH8

1st Step: remove ethanol from the plate.

2nd Step: wash the plate with 400 μl of milliQ H2O.

3rd Step: equilibrate the plate with 400 μl of Buffer A

4th Step: load 600 μl of starting material for each protein in one of the 12 columns. If the volume is larger, repeat until all the material is fully loaded.

Recover the flow through.

5th Step: Wash Step: 4 washes with 400 μl of Buffer C. Discard the flow through.

6th Step: Elution: 2×300 μl Buffer B (2 elution steps).

Activate vacuum 15 minutes after adding the buffer.

1 μl of total extract, 1 μl of starting material, 1 μl of flow through and 10μ1 of elution volume (for each protein) are analysed by SDS-PAGE.

For insoluble protein, buffer B is replaced by 10 mM tris, 50 mM Na₂HPO₄, 8M urea, 250 mM imidazole, 40% glycerol.

LAL Test

The LAL test is a test that measures the endotoxin concentration in a vaccine sample using the Endosafe®-PTS™ Charles River technology.

Test Technology

The PTS utilizes LAL kinetic chromogenic methodology to measure color intensity directly related to the endotoxin concentration in a sample. Each cartridge contains precise amounts of licensed LAL reagent, chromogenic substrate, and control standard endotoxin (CSE). The cartridges are manufactured according to rigid quality control procedures to ensure test accuracy and product stability.

TABLE II
Purification of preferred antigens

| | | | kDa | | | Purity % | | | LAL |
|---|---|---|---|---|---|---|---|---|---|
| Internal | | kDa | (SE | | | RP- | SE- | | Test |
| ID | Annotation | (expected) | estimated) | Soluble | densitometry | HPLC | UPLC | SE-HPLC | EU/μg |
| nt001 | NTHI0877 | 30 | 36 | yes | 97 | 84 | | monomer | 0.47 |
| nt018 | NTHI0915 | 34 | 46 | yes | 80 | 88 | | monomer | 0.18 |
| nt024 | NTHI1416 | 20 | 20 | yes | 81 | 85 | 97 | monomer | 3.77 |
| nt032 | NTHI2017 | 13 | 16 | yes | 91 | 76 | | monomer | 0.82 |
| nt067 | NTHI1292 | 60 | 50 | yes | 88 | 78 | | monomer | 0.06 |
| nt052 | CGSHiGG_00130 | 34 | 46 | yes | 88 | 88 | | monomer | 0.18 |
| nt004 | CGSHiGG_08215 | 20 | 34 | yes | 95 | | 95 | monomer | 0.09 |
| nt014 | HI1658 | 20 | 17 | yes | 89 | | 87 | monomer | 0.10 |
| nt022 | NTHI0830 | 43 | 77 | yes | 93 | | 93 | monomer | 0.10 |
| nt016 | NTHI0266 | 29 | 30 | yes | 98% | | | monomer | 0.13 |

Immunisation of Mice and Production of Antisera

Five weeks old CD1 mice (8 for each antigen) were immunized by 3 intraperitoneal injections (every two weeks) of 10 micrograms of purified protein antigens with Freund's adjuvant (200 microliters per mouse) or with Alum (aluminium hydroxide adjuvant; 2 mg/ml). Sera were collected two weeks after the third injection and stocked at −20° C. Controls were injected with Freund's adjuvant only or alum only.

FACS Analysis

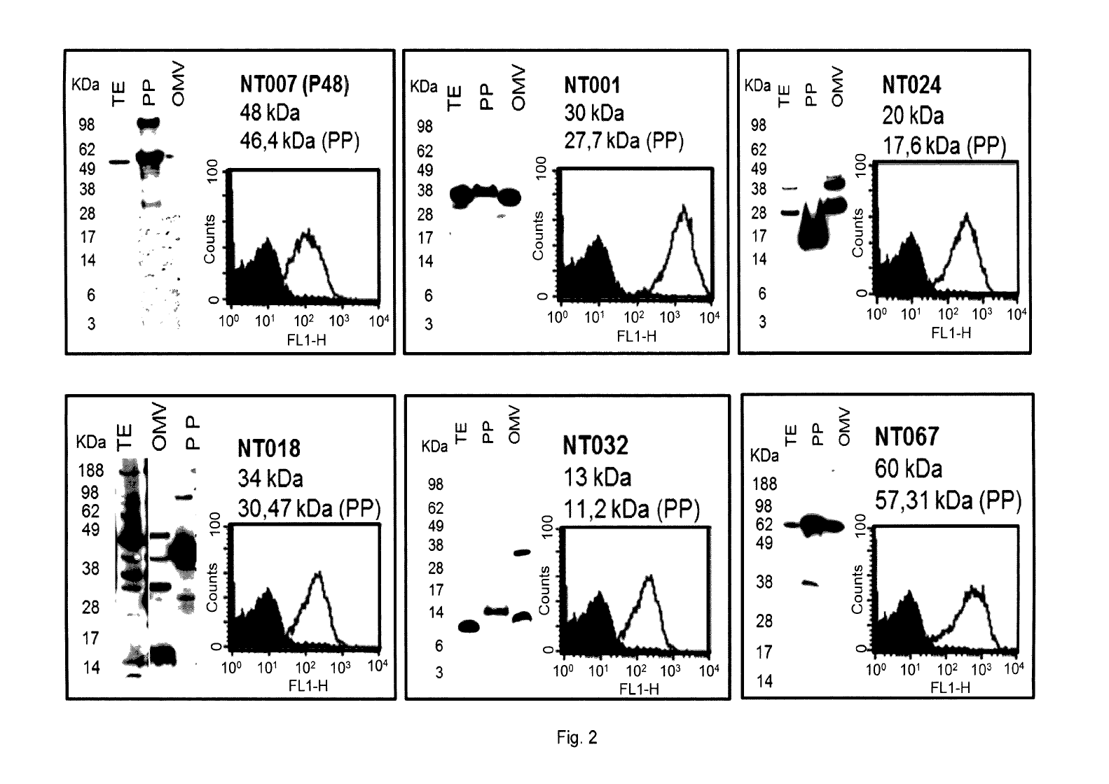

A surface labeling assay by FACS was performed in order to examine the surface exposure of the selected antigens and the levels of expression in different strains. NTHI were incubated with sera derived from mice immunized with recombinant proteins or negative controls, and analysed by FACS. The results are shown in FIG. 2. In FIG. 2, pre-immune serum negative controls are shown as solid areas, and the signal obtained with sample serum is shown as a single line. The results of FACS analyses of antigens P48, HtrA, PE, and P26 demonstrate that each of these antigens is exposed on the surface of the bacterium and thus accessible to antibody binding.

The following materials and methods were used in this analysis:

### Materials

1. 96 U-bottom well plates.
2. Blocking and Washing Buffer: PBS containing 1% (w/v) BSA.
3. Goat anti-mouse IgG-Fluorescein IsoThio Cyanate FITC.
4. PBS containing 0.5% (v/v) para-formaldehyde: dilute a stock solution of 4% (v/v) para-formaldehyde in PBS to 0.5% (v/v) fresh before the assay and filter sterilize (0.22 μm filter).
5. PBS containing 1% (w/v) BSA. To prepare this solution, dissolve 1% (w/v) BSA in PBS, making at least 100 ml for each strain. Filter-sterilize the solution (0.22 μm filter) and prepare fresh for use.
6. FACScan tubes (Becton Dickson).
7. FACScalibur flow cytometer (Becton Dickinson).

Methods
1. Grow NTHI until an ODλ₆₀₀ nm value of 0.5 is reached, then transfer 1 ml of culture to a sterile 1.5 ml Eppendorf tube and centrifuge at 13000 g in a micro-centrifuge for 3 minutes to pellet the bacteria. Discard the supernatant and suspend the pellet suspended in 1 ml of PBS containing 1% (w/v) BSA. Finally, dilute the bacterial suspension 1/50 in PBS containing 1% (w/v) BSA.
2. Add 500 samples of sera diluted in Blocking Buffer (at 1/100, 1/200 and 1/400) in a 96 well plate. Include positive controls, such as anti-OMV antisera,
3. Add 50 μl of bacterial cells to each well and store the plate at 4° C. for 2 h.
4. Centrifuge the cells for 5 minutes at 3500 g, discard the supernatant and wash the cells by adding 2000/well of Washing Buffer.
5. Add 500 of a 1/100 dilution of FITC-conjugated goat anti-mouse Ig to each well and store the plate at 4° C. for 1 h.
6. Centrifuge the cells at 3500 g for 5 min and wash the pellet with 2000/well of PBS.
7. Repeat the centrifugation step, discard the supernatant and add 2000/well of PBS containing 0.5% (v/v) para-formaldehyde, in order to fix the cells.
8. Transfer the fixed samples to individual FACScan tubes and analyse by flow cytometry, following the equipment manufacturer's instructions.

Serum Bactericidal Assay (SBA)

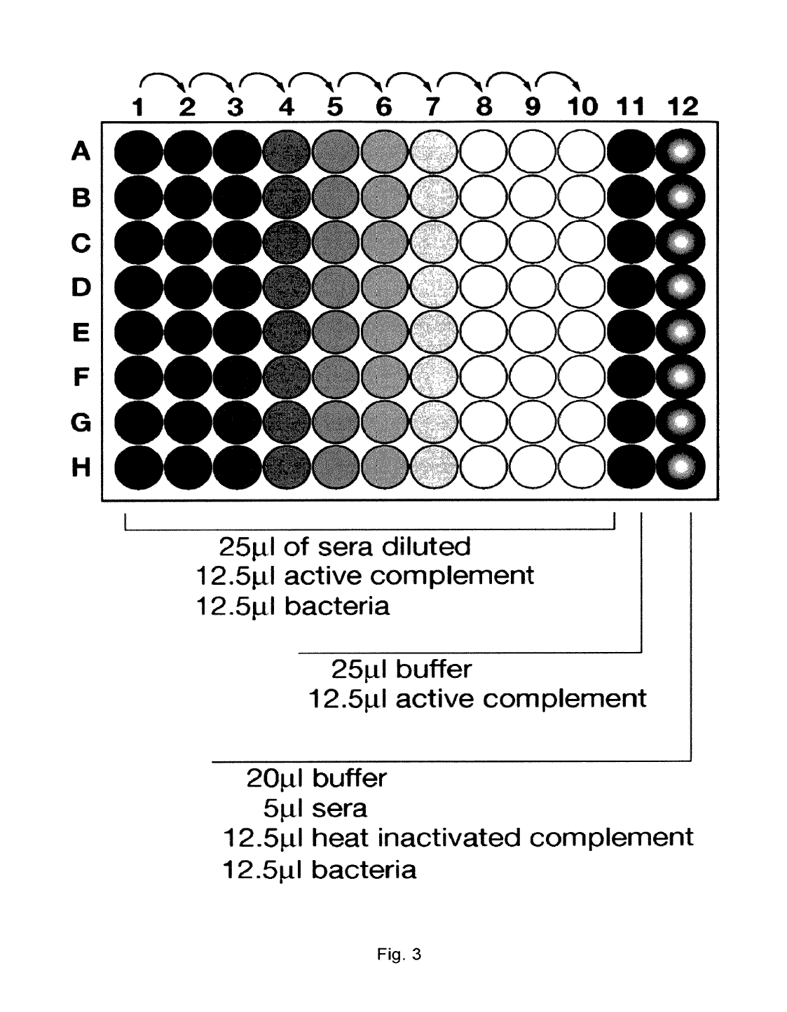

Antisera derived from mice immunized with recombinant proteins were tested in a serum bactericidal assay, to verify the presence of functional antibodies able to induce killing of NTHI. Pre-immune sera and sera from mice injected only with adjuvant were used as negative controls. NTHI (strain 176) culture (BHI+NAD and Haemin) was incubated at 37° C. with shaking, until OD595 nm was 0.25-0.27. The bacterial cells were diluted in D-PBS buffer at the working dilution 1:50000. Sera were inactivated at 56° for 30 minutes and then serially diluted in D-PBS in a 96-well U-bottom plate (see FIG. 3). Columns 11 and 12 of the plate shown in FIG. 3 contain negative controls to assess the growth of the bacteria and to detect any non-complement mediated killing. Bacteria and a source of complement (Rabbit 7504, Cedarlane) were added to each well except in the complement control wells which received heat-inactivated complement.

As shown in FIG. 3, wells in columns 1-10 contain 25 μl diluted sera, 12.5 μl active complement, and 12.5 μl bacteria. Wells in column 11 contain 25 μl buffer, 12.5 μl active complement, and 12.5 μl bacteria. Wells in column 12 contain 25 μl buffer, 5 μl sera, 12.5 μl heat inactivated complement, and 12.5 μl bacteria.
10 μl of the time zero (TO) assay controls (column 11-12) were plated on agar chocolate plate (Biomerieux) by the spot and tilt method. Plates were incubated at 37° C., ON. The assay microtiter plates were incubated for 1 hour at 37° C. After this period (T60) 7 μl of each well were plated as spot on an agar chocolate plate (each well was plated in duplicate). The number of colonies (colony forming units, CFU) was counted using a colony counter or manually. A bactericidal effect was considered to be observed when the number of colonies was lower than 50% of T=0.

An overview of the results is provided in the following Table III and Table IV.

TABLE III
Immunogenicity results

| | | | | SBA TITER Freund's | |
|---|---|---|---|---|---|
| Internal ID | Annotation | SEq ID NOs | kDa | adjuvant (176wt) | FACS |
| NT001 | NTHI0877 | SEQ ID NO: 6 or | 30 | 2048-8192 | +++++ |
| | | SEQ ID NO: 54 | | | |
| NT016 | NTHI0266 | SEQ ID NO: 7 or | 29 | 2048-8192 | +++++ |
| | | SEQ ID NO: 55 | | | |
| NT024 | NTHI1416 | SEQ ID NO: 2 or | 20 | 2048-8192 | ++ |
| | | SEQ ID NO: 50 | | | |
| NT032 | NTHI2017 | SEQ ID NO: 3 or | 13 | 2048-8192 | + |
| | | SEQ ID NO: 51 | | | |
| NT018 | NTHI0915 | SEQ ID NO: 1 or | 34 | 2048-4096 | + |
| | | SEQ ID NO: 49 | | | |
| NT038 | CGSHiGG_ | SEQ ID NO: 4 or | 22 | 2048-4096 | ++ |
| | 02400 | SEQ ID NO: 50 | | | |
| NT052 | CGSHiGG_ | SEQ ID NO: 8 or | 44 | 2048-4096 | ++ |
| | 00130 | SEQ ID NO: 56 | | | |
| NT067 | NTHI1292 | SEQ ID NO: 5 or | 60 | 2048 | ++ |
| | | SEQ ID NO: 52 | | | |
| NT002 | NTHI1627 | SEQ ID NO: 9 or | 18 | 1024-2048 | +++ |
| | | SEQ ID NO: 57 | | | |
| NT026 | NTHI1109 | SEQ ID NO: 10 or | 19 | 4096-8192 | ++++ |
| | | SEQ IDNO: 58 | | | |
| NT009 | NTHI0821 | SEQ ID NO: 11 or | 64 | 4096 | +++ |
| | | SEQ ID NO: 59 | | | |
| NT025 | NTHI0409 | SEQ ID NO: 12 or | 17 | 4096 | ++++ |
| | | SEQ IDNO: 60 | | | |
| NT028 | NTHI1954 | SEQ ID NO: 13 | 20 | 4096 | +++ |
| | | SEQ ID NO: 61 | | | |
| NT029 | NTHI0371 | SEQ ID NO: 14 | 101 | 4096 | +++ |
| | | SEQ ID NO: 62 | | | |
| NT031 | NTHI0509 | SEQ ID NO: 15 | 20 | 4096 | + |
| | | SEQ ID NO: 63 | | | |
| NT015 | NTHI0449 | SEQ ID NO: 16 | 15 | 2048-4096 | ++ |
| | | SEQ ID NO: 64 | | | |
| NT023 | NTHI1473 | SEQ ID NO: 17 | 17 | 2048-4096 | ++ |
| | | SEQ ID NO: 65 | | | |
| NT100 | gi145633184 | SEQ ID NO: 18 | 34 | 2048-4096 | + |
| | | SEQ ID NO: 66 | | | |
| NT040 | NTHI1110 | SEQ ID NO: 19 | 26 | 1024-2048 | + |
| NT048 | gi-46129075 | SEQ ID NO: 20 | 71 | 1024-2048 | + |
| NT053 | gi145628236 | SEQ ID NO: 21 | 17 | 1024-2048 | + |
| NT066 | NTHI1230 | SEQ ID NO: 22 | 59 | 1024-2048 | + |
| | | SEQ ID NO: 67 | | | |
| NT097 | NTHI0522 | SEQ ID NO: 23 | 50 | 1024-2048 | ++ |
| NT006 | NTHI1905 | SEQ ID NO: 25 | 51 | 2048 | ++++ |
| (HtrA) | | | | | |
| NT035 (PE) | NTHI0267 | SEQ ID NO: 26 | 18 | 512-1024 | ++ |
| NT080 | NTHI0811 | SEQ ID NO: 28 | | 512 | |
| (PHiD) | | | | | |
| NT081(P6) | NTHI0501 | SEQ ID NO: 29 | | 512 | |
| NT010 (P26) | NTHI1083 | SEQ ID NO: 27 | 22 | 128-512 | +++ |
| NT007 (P48) | NTHI0254 | SEQ ID NO: 24 | 48 | 8192-16384 | +++++ |
| Unrelated | | | | 16 | + |
| antigen | | | | | |
| Freund's | | | | 512/1024 | |
| Adj. alone | | | | | |

These results show that antigens selected are highly effective in killing NTHI pathogens. In particular NT018, NT001, NT024, NT032, NT067, NT016 all show particularly strong protective effects.

TABLE IV
Immunization experiments using compositions comprising
NTHI antigens and Alum

| | | SBA | | FACS | FACS | |
|---|---|---|---|---|---|---|
| Protein Antigen | Purity | (Freund) | SBA (Alum) | Freund | alum | Solubility |
| NT001 | 97% | 2048-8192 | 512-1024 | +++++ | ++ + | Yes |
| NT024 | 94% | 2048-8192 | 1024 | ++ | ++ + | Yes |
| NT038 | 97% | 2048-4096 | 64 | ++ | − | Yes |
| NT018 | 80% | 2048-4096 | 512-1024 | + | +++ | Yes |
| NT032 | 99% | 2048-8192 | 64-128 | + | + | Yes |
| NT067 | 88% | 2048 | 512-2048 | ++ | +++ | Yes |
| NT025 | 94% | 4096 | 128-256 | ++++ | + | No |
| NT026 | 64% | 4096-8192 | 64 | ++++ | − | No |
| NT028 | 81% | 4096 | 128-256 | +++ | ++ | No |
| NT029 | 52% | 4096 | 128 | +++ | ++ | Yes |
| NT023 | 80% | 2048-4096 | 256-512 | ++ | + | Yes |
| NT015 | 78% | 2048-4096 | 128-256 | ++ | + | No |
| NT031 | 90% | 4096 | 128 | + | + | No |
| NT100 | 81% | 2048-4096 | 512 | + | + | Yes |
| NT081 (P6) | 88% | 2048 | 256 | + | + | No |
| NT080 (PHiD) | 92% | 1024 | 128 | + | + | |
| NT006 (HtrA) | 57% | 2048 | 256 | ++++ | ++++ | |
| NT007 (P48) | 79% | 8192-16384 | 256-512 | +++++ | +++++ | |
| NT052 | 88% | 2048-4096 | 512-1024 | + | +++ | Yes |
| NT014 | 87% | 1024 | 512-1024 | ++ | ++ | Yes |
| NT004 | 95% | 256-512 | 128-256 | ++ | + | Yes |
| NT022 | 93% | 64-256 | 1024 | +++ | + | Yes |
| NT016 | 98% | 2048-8192 | 128 | +++ | ++++ | Yes |
| NT106 | 82% | Not tested | 64-128 | ++ | +++ | Yes |
| NT113 | 92% | Not tested | 128 | ++ | +++ | Yes |
| NT061 | 83% | Not tested | 128 | +++ | +++ | Yes |
| Freund's | | 512 | NA | | | |
| Alum | | NA | 4-8 | | | |

These results further confirmed that antigens selected are highly effective in killing NTHI pathogens also when used in immunogenic compositions with alum as adjuvant.

In particular NT016, NT052, NT018, NT001, NT024, NT032, NT067, NT014, NT022 all confirm particularly strong protective effects as measured in serum bactericidal assay (SBA).

Particularly preferred antigens were NT067, NT014, NT016, NT022. These antigens have been also tested in an in vivo animal model according to the protocol described in Ref 75.

In Vivo Vaccine Efficacy Testing

Individual antigens as listed in Table IV can be tested for their ability to protect against an otitis media OM) infection using an in vivo model such as Junbo and Jeff mouse mutants [75].

The vaccine efficacy in the in vivo protection experiment is performed using 3 administrations (at day 0, 21, 35) of 10 micrograms/mouse of purified recombinant protein antigens formulated with or without adjuvant, followed by intranasal inoculation with selected NTHI pathogenic strains.

Pre-immune sera, post-immunization sera, and terminal sera 7 days post-NTHI inoculation are collected and stored at −80° C. Controls are immunized with adjuvant or with an unrelated antigen as control. Middle ear bulla and nasopharyngeal (NP) washes samples are collected and plated to determine NTHi numbers; bulla infection and nasopharingeal carriage rates, and bulla NTHi titres are then calculated.

It will be understood that the invention is described above by way of example only and modifications may be made whilst remaining within the scope and spirit of the invention.

TABLE V
Nomenclature cross-reference with representative strains

| | | 86-028NP | | |
|---|---|---|---|---|
| SEQ ID NOs | Name | NTHI_# | 3655 Strain | PittG Strain |
| 1 or 49 | NT018 | NTHI0915 | | |
| 2 or 50 | NT024 | NTHI1416 | | |
| 3 or 51 | NT032 | NTHI2017 | | |
| 4 or 53 | NT038 | | | CGSHiGG_02400 |
| 5 or 52 | NT067 | NTHI1292 | | |
| 6 or 54 | NT001 | NTHI0877 | | |
| 7 or 55 | NT016 | NTHI0266 | | |
| 8 or 56 | NT052 | | | CGSHiGG_00130 |
| 9 or 57 | NT002 | NTHI1627 | | |
| 10 or 58 | NT026 | NTHI1109 | | |
| 11 or 59 | NT009 | NTHI0821 | | |
| 12 or 60 | NT025 | NTHI0409 | | |
| 13 or 61 | NT028 | NTHI1954 | | |
| 14 or 62 | NT029 | NTHI0371 | | |
| 15 or 63 | NT031 | NTHI0509 | | |
| 16 or 64 | NT015 | NTHI0449 | | |
| 17 or 65 | NT023 | NTHI1473 | | |
| 18 or 66 | NT100 | | gi-145633184 | |
| 19 | NT040 | NTHI1110 | | |
| 20 | NT048 | | gi-46129075 | |
| 21 | NT053 | | gi-145628236 | |
| 22 or 67 | NT066 | NTHI1230 | | |
| 23 | NT097 | NTHI0522 | | |
| 24 | NT007 | P48 | | |
| 25 | NT006 | HtrA | | |
| 26 | NT035 | PE | | |
| 27 | NT010 | P26 | | |
| 28 | NT080 | PHiD | | |
| 29 | NT081 | P6 | | |
| 30 | NT013 | NTHI0532 | | |
| 31 | NT106 | NTHI0363 | | |
| 32 | NT107 | NTHI0370 | | |
| 33 | NT108 | NTHI0205 | | |
| 34 | NT109 | NTHI0374 | | |
| 35 | NT110 | NTHI0579 | | |
| 36 | NT111 | NTHI0837 | | |
| 37 | NT112 | NTHI0849 | | |
| 38 | NT113 | NTHI0921 | | |
| 39 | NT114 | NTHI0995 | | |
| 40 | NT115 | NTHI1091 | | |
| 41 | NT116 | NTHI1169 | | |
| 42 | NT117 | NTHI1208 | | |
| 43 | NT118 | NTHI1318 | | |
| 44 | NT123 | NTHI1796 | | |
| 45 | NT124 | NTHI1930 | | |
| 114 | NT119 | NTHI1565 | | |
| 115 | NT120 | NTHI1569 | | |
| 116 | NT121 | NTHI1571 | | |
| 117 | NT122 | NTHI1667 | | |
| 122 | NT004 | | | CGSHiGG_08215 |
| 123 | NT014 | | gi-145629254 | |
| 124 | NT022 | NTHI0830 | | |
| 128 | NT061 | NTHI0588 | | |
| 130 | NT017 | NTHI0915 | | |

### REFERENCES

[1] Fleischmann et al. (1995) Science 269:496-512.
[2] Li et al. (2003) Mol Microbiol 47:1101-1111.
[3] GenBank accession NC_000907.
[4] WO2005/111066
[5] Murphy et al. Current Infectious Disease report (2009)11:177-182.
[6] Webb D C et al.—Investigation of the potential of a 48 kDa protein as a vaccine candidate for infection against nontypable Haemophilus influenzae. Vaccine. 2007 May 16; 25(20):4012-9.
[7] Hallström T et al.—Nontypeable Haemophilus influenzae protein E binds vitronectin and is important for serum resistance. J Immunol. 2009 Aug. 15; 183(4):2593-601.
[8] Loosmore S M et al.—The Haemophilus influenzae HtrA protein is a protective antigen. Infect Immun. 1998 March; 66(3):899-906.
[9] Kyd J M et al.—Potential of a novel protein, OMP26, from nontypeable Haemophilus influenzae to enhance pulmonary clearance in a rat model. Infect Immun. 1998 May; 66(5):2272-8.
[10] Ronander E, The Journal of Infectious Diseases (2009): 199 p522-530
[11] WO00/55191.
[12] WO02/24729.
[13] Chanyangam M. et al., (1991) Infection and Immunity, Vol. 59 (2), 600-608
[14] Munson R. S.; Granoff D. M (1985) Infection and Immunity 49 (3):544-549
[15] Hogg et al. (2007) Genome Biology 8:R103.
[16] Needleman & Wunsch (1970) J. Mol. Biol. 48, 443-453.
[17] Rice et al. (2000) Trends Genet 16:276-277.
[18] Bernadac A., et al. (1998) Journal of Bacteriology 180 (18): 4872-4878
[19] WO2002/062378
[20] Uehara T. et al., The EMBO Journal (2010) 29, 1412-1422
[21] U.S. Pat. No. 5,707,829
[22] Current Protocols in Molecular Biology (F. M. Ausubel et al. eds., 1987) Supplement 30.
[23] Vaccine Design (1995) eds. Powell & Newman. ISBN: 030644867X. Plenum.
[24] WO90/14837.
[25] Podda & Del Giudice (2003) Expert Rev Vaccines 2:197-203.
[26] Podda (2001) Vaccine 19: 2673-2680.
[27] Vaccine Adjuvants: Preparation Methods and Research Protocols (Volume 42 of Methods in Molecular Medicine series). ISBN: 1-59259-083-7. Ed. O'Hagan.
[28] U.S. Pat. No. 5,057,540.
[29] Niikura et al. (2002) Virology 293:273-280.
[30] Lenz et al. (2001) J Immunol 166:5346-5355.
[31] Pinto et al (2003) J Infect Dis 188:327-338.
[32] Gerber et al. (2001) J Virol 75:4752-4760.
[33] WO03/024480.
[34] WO03/024481.
[35] Gluck et al. (2002) Vaccine 20:B10-B16.
[36] Meraldi et al. (2003) Vaccine 21:2485-2491.
[37] Pajak et al. (2003) Vaccine 21:836-842.
[38] Krieg (2003) Nature Medicine 9:831-835.
[39] McCluskie et at (2002) FEMS Immunology and Medical Microbiology 32:179-185.
[40] WO98/40100.
[41] U.S. Pat. No. 6,207,646.
[42] U.S. Pat. No. 6,239,116.
[43] U.S. Pat. No. 6,429,199.
[44] Schellack et al. (2006) Vaccine 24:5461-72.
[45] Johnson et al. (1999) Bioorg Med Chem Lett 9:2273-2278.
[46] Evans et al. (2003) Expert Rev Vaccines 2:219-229.
[47] Beignon et al. (2002) Infect Immun 70:3012-3019.
[48] Pizza et al. (2001) Vaccine 19:2534-2541.
[49] Pizza et al. (2000) Int J Med Microbiol 290:455-461.
[50] Scharton-Kersten et al. (2000) Infect Immun 68:5306-5313.
[51] Ryan et al. (1999) Infect Immun 67:6270-6280.
[52] Partidos et al. (1999) Immunol Lett 67:209-216.
[53] Peppoloni et al. (2003) Expert Rev Vaccines 2:285-293.
[54] Pine et al. (2002) J Control Release 85:263-270.
[55] WO99/40936.
[56] WO99/44636.
[57] Singh et al] (2001) J Cont Release 70:267-276.
[58] WO99/27960.
[59] U.S. Pat. No. 6,090,406.
[60] U.S. Pat. No. 5,916,588.
[61] EP-A-0626169.
[62] WO99/52549.
[63] Andrianov et al. (1998) Biomaterials 19:109-115.
[64] Payne et al. (1998) Adv Drug Delivery Review 31:185-196.
[65] Stanley (2002) Clin Exp Dermatol 27:571-577.
[66] Jones (2003) Curr Opin Investig Drugs 4:214-218.
[67] WO99/11241.
[68] WO94/00153.
[69] WO98/57659.
[70] European patent applications 0835318, 0735898 and 0761231.
[71] Ogunniyi et al. (2001) Infect Immun 69:5997-6003.
[72] WO2006/110603.
[73] Mason et al. (2003) Infect Immun 71:3454-3462.
[74] Zwijnenburg et al. (2001) J Infect Dis 183:1143-6.
[75] Cheeseman M. T. et al. (2011) PLoS Genetics 7 (10): e1002336.
[76] Watson (2000) Pediatr Infect Dis J 19:331-332.
[77] Rubin (2000) Pediatr Clin North Am 47:269-285, v.
[78] Jedrzejas (2001) Microbiol Mol Biol Rev 65:187-207.
[79] Bell (2000) Pediatr Infect Dis J 19:1187-1188.
[80] Iwarson (1995) APMIS 103:321-326.
[81] Gerlich et al. (1990) Vaccine 8 Suppl:S63-68 & 79-80.
[82] Vaccines (1988) eds. Plotkin & Mortimer. ISBN 0-7216-1946-0.
[83] Del Guidice et al. (1998) Molecular Aspects of Medicine 19:1-70.
[84] Gustafsson et al. (1996)N. Engl. J. Med. 334:349-355.
[85] Rappuoli et al. (1991) TIBTECH 9:232-238.
[86] Costantino et al. (1999) Vaccine 17:1251-1263.
[87] Sutter et al. (2000) Pediatr Clin North Am 47:287-308.
[88] Zimmerman & Spann (1999) Am Fam Physician 59:113-118, 125-126.
[89] McMichael (2000) Vaccine 19 Suppl 1:S101-107.
[90] Schuchat (1999) Lancet 353(9146):51-6.
[91] WO02/34771.
[92] Dale (1999) Infect Dis Clin North Am 13:227-43, viii.
[93] Ferretti et al. (2001) PNAS USA 98: 4658-4663.
[94] Kuroda et al. (2001) Lancet 357(9264):1225-1240; see also pages 1218-1219.
[95] EP-A-0372501
[96] EP-A-0378881
[97] EP-A-0427347
[98] WO93/17712
[99] WO94/03208
WO98/58668
EP-A-0471177
WO00/56360
WO91/01146
WO00/61761
WO01/72337
Research Disclosure, 453077 (January 2002)
Brandt et al. (2006) J Antimicrob Chemother. 58(6):1291-4. Epub 2006 Oct. 26
Winter et al., (1991) Nature 349:293-99
U.S. Pat. No. 4,816,567.
Inbar et al., (1972) Proc. Natl. Acad. Sci. U.S.A. 69:2659-62.
Ehrlich et al., (1980) Biochem 19:4091-96.
Huston et al., (1988) Proc. Natl. Acad. Sci. U.S.A. 85:5897-83.
Pack et al., (1992) Biochem 31, 1579-84.
Cumber et al., (1992) J. Immunology 149B, 120-26.
Riechmann et al., (1988) Nature 332, 323-27.
Verhoeyan et al., (1988) Science 239, 1534-36.
GB 2,276,169.
Gennaro (2000) Remington: The Science and Practice of Pharmacy. 20th edition, ISBN: 0683306472.
Methods In Enzymology (S. Colowick and N. Kaplan, eds., Academic Press, Inc.)
Handbook of Experimental Immunology, Vols. I-IV (D. M. Weir and C. C. Blackwell, eds, 1986, Blackwell Scientific Publications)
Sambrook et al. (2001) Molecular Cloning: A Laboratory Manual, 3rd edition (Cold Spring Harbor Laboratory Press).
Handbook of Surface and Colloidal Chemistry (Birdi, K. S. ed., CRC Press, 1997)
Ausubel et al. (eds) (2002) Short protocols in molecular biology, 5th edition (Current Protocols).
Molecular Biology Techniques: An Intensive Laboratory Course, (Ream et al., eds., 1998, Academic Press)
PCR (Introduction to Biotechniques Series), 2nd ed. (Newton & Graham eds., 1997, Springer Verlag)
Geysen et al (1984) PNAS USA 81:3998-4002.
Carter (1994) Methods Mol Biol 36:207-23.
Jameson, B A et al 1988, CABIOS 4(1):181-186.
Raddrizzani & Hammer (2000) Brief Bioinfonn 1(2):179-89.
Bublil et al. (2007) Proteins 68(1):294-304.
De Lalla et al. (1999) J. Immunol. 163:1725-29.
Kwok et al. (2001) Trends Immunol 22:583-88.
Brusic et al. (1998) Bioinformatics 14(2):121-30
Meister et al. (1995) Vaccine 13(6):581-91.
Roberts et al. (1996) AIDS Res Hum Retroviruses 12(7):593-610.
Maksyutov & Zagrebelnaya (1993) Comput Appl Biosci 9(3):291-7.
Feller & de la Cruz (1991) Nature 349(6311):720-1.
Hopp (1993) Peptide Research 6:183-190.
Welling et al. (1985) FEBS Lett. 188:215-218.
Davenport et al. (1995) Immunogenetics 42:392-297.
Tsurui & Takahashi (2007) J Pharmacol Sci. 105(4):299-316.
Tong et al. (2007) Brief Bioinform. 8(2):96-108.
Schirle et al (2001) J Immunol Methods. 257(1-2):1-16.
Chen et al. (2007) Amino Acids 33(3):423-8.
Smith & Waterman (1981) Adv. Appl. Math. 2: 482-489.
Lundström et al. (2008) Biochemistry, 47 (22): 6025-38 Structural analysis of the lipopolysaccharide from nontypeable Haemophilus influenzae strain R2846.
Klock, H. E., et al. (2008). Proteins 71:982-994
Klock, H. E., et al. (2005) J. Struct. Funct. Genomics 6, 89-94

1. An immunogenic composition comprising one or more non-typeable H. influenzae isolated or recombinant polypeptide antigens, selected from the group consisting of: (5) NTHI1292 (NT067), (8) CGSHiGG_00130 (NT052), (1) NTHI0915 (NT018), (2) NTHI1416 (NT024), (3) NTHI2017 (NT032), (4) CGSHiGG_02400 (NT038), (6) NTHI0877 (NT001), (7) NTHI0266 (NT016), (9) NTHI1627 (NT002), (10) NTHI1109 (NT026), (11) NTHI0821 (NT009), (12) NTHI0409 (NT025), (13) NTHI1954 (NT028), (14) NTHI0371 (NT029), (15) NTHI0509 (NT031), (16) NTHI0449 (NT015), (17) NTHI1473 (NT023), (18) gi-145633184 (NT100), (19) NTHI1110 (NT040), (20) gi-46129075 (NT048), (21) gi-145628236 (NT053), (22) NTHI1230 (NT066), (23) NTHI0522 (NT097), (24) NT004, (25) NT014, (26) NT022.

2. The composition of claim 1 wherein,
said NT067 antigen is a polypeptide that comprises an aminoacid sequence: (a) having 80% or more identity to SEQ ID NO: 5 or to SEQ ID NO: 52; and/or (b) that is a fragment of at least 10 consecutive aminoacids, and comprises an epitope, of SEQ ID NO: 5 or SEQ ID NO: 52;
said NT016 antigen is a polypeptide that comprises an amino acid sequence: (a) having 80% or more identity to SEQ ID NO: 7 or SEQ ID NO: 55; and/or (b) that is a fragment of at least 10 consecutive amino acids, and comprises an epitope, of SEQ ID NO: 7 or SEQ ID NO: 55;
said NT014 antigen is a polypeptide that comprises an amino acid sequence: (a) having 80% or more identity to SEQ ID NO: 123; and/or (b) that is a fragment of at least 10 consecutive amino acids, and comprises an epitope, of SEQ ID NO: 123;
said NT022 antigen is a polypeptide that comprises an amino acid sequence: (a) having 80% or more identity to SEQ ID NO: 124; and/or (b) that is a fragment of at least 10 consecutive amino acids, and comprises an epitope, of SEQ ID NO: 124.

3. An immunogenic composition according to claim 1, further comprising at least one polypeptide selected from the group consisting of: (24) P48 (NTHI0254 also defined as NT007), (25) HtrA (NTHI1905 also defined as NT006), (26) PE (NTHI0267 also defined as NT035), (27) P26 (NTHI0501 also defined as NT010), (28) PHiD (NTHI0811 also defined as NT080), (29) P6 (NTHI0501, also defined as NT081).

4. An immunogenic composition according to claim 1, further comprising at least one polypeptide selected from the group consisting of: (30) NT013, (31) NT106, (32) NT107, (33) NT108, (34) NT109, (35) NT110, (36) NT111, (37) NT112, (38) NT113, (39) NT114, (40) NT115, (41) NT116, (42) NT117, (43) NT118, (44) NT123, (45) NT124, (46) NT119, (47) NT120, (48) NT121, (49) NT122 and/or (50) NT061.

5. An immunogenic composition according to claim 1 comprising a combination of two or more antigens.

6. The composition according to claim 1, wherein the at least one of non-typeable H. influenzae isolated or recombinant polypeptide antigens is conserved amongst different pathogenic non-typeable H. influenzae strains.

7. The composition according to claim 1, further comprising at least one vaccine antigen that is not a non-typeable H. influenzae antigen selected from any of:
an antigen from N. meningitidis serogroup A, B, C, W135 and/or Y.
a saccharide or polypeptide antigen from Streptococcus pneumoniae
an antigen from hepatitis A virus
an antigen from hepatitis B virus
a diphtheria antigen, such as a diphtheria toxoid
a tetanus antigen
an antigen from Bordetella pertussis
a whole cellular pertussis antigen
a saccharide antigen from Haemophilus influenzae B
polio antigen(s)
measles, mumps and/or rubella antigens
influenza antigen(s)
an antigen from Moraxella catarrhalis
an antigen from Respiratory Syncytial Virus
a vaccine composition comprising diphtheria (D), tetanus (T), pertussis (acellular, component) (Pa), hepatitis B (rDNA) (HBV), poliomyelitis (inactivated) (IPV) and Haemophilus influenzae type b (Hib) conjugate vaccine (adsorbed), e.g. Infanrix-hexa

8. The composition according to claim 1 which further comprises one or more pharmaceutically acceptable carriers, diluents and/or adjuvants.

9. The composition according to claim 1 for use in medicine.

10. The composition of claim 1, wherein said composition is a vaccine.

11. The composition of claim 1 for use as immunizing agent against Haemophilis influenzae sp.

12. The composition of claim 1 for use as immunizing agent against Non-typeable Haemophilus influenzae caused infection, such as otitis media.

13. The composition of claim 1 for use as vaccine against Haemophilus influenzae caused diseases.

14. A method for preventing or treating infections by non-typeable H. influenzae, comprising the step of administering to the mammal an effective amount of the composition of claim 1, or comprising one or more steps of administering at least one antigen of said composition.

15. The method of claim 13 comprising a step wherein at least one antigen are administered simultaneously.

16. The method of claim 13, wherein at least one antigen are administered separately in more than one step.

17. At least one antigens as defined in claim 1, for combined use in raising an immune response in a mammal.

18. A process for preparing a composition as defined in claim 1, comprising a step of mixing one or more antigens with an adjuvant.

19. The process of claim 18, further comprising a step of formulating the mixture as a medicament or vaccine, and optionally further comprising a step of subsequently packaging the formulation for distribution as a medicament or vaccine.


SEQUENCE LISTING (131 sequences)

SEQ ID NO: 1 - protein, 277 aa, Unknown
  Description of Unknown Bacteria <Prokaryote> sequence
  NT018
KHGQKRDDLNKALYFSRLEEIEQDNSQGLVENVEQLKQELQKTLLDDVPSKVQENVDYSGKSYGKIWFVSGVLALGIIAGSSYFMVGSWQAESMLEQTYAKLPYFFDRMKDEDKNPFSDAEMQQFSIALRIDLQKNPTDAKKWWMLGQIGMNLGDARLAFDSYQKANKLEPDNVQYKLGYARILMFSEDATDKLKGGNLLREVIRQEHTNIEALSLLAFRYFETEDYKMAAVTWAMMLRLMPKDDERVPLIEKSIRTARDALEAQNEEKSKSITPEK

SEQ ID NO: 2 - protein, 160 aa, Unknown
  Description of Unknown Bacteria <Prokaryote> sequence
  NT024
MKTIDITANSKMDDQARMNLAQEFANKQQWSSVFDIMYPMALEGNTTAQSNLGMLYNLGRGTVRDYEKAYWWFSEAAEKGSVKGLNNLGVMYLRGDYVKQNTEQAIKLFERTARAKDTDAMMMLSNIYRLQNQPEKSLEWLKKAAELGNKEAKQRLSSQP

SEQ ID NO: 3 - protein, 102 aa, Unknown
  Description of Unknown Bacteria <Prokaryote> sequence
  NT032
GFNGNNSQGGFQQTAPAAISVKQALSAADNSMITLVGNITQQIDDDEFWFTDGTGQIKIEIKKRVWNGLNVDSKDKVKIYGKLDNEAFEKAELDVLRVEKAE

SEQ ID NO: 4 - protein, 259 aa, Unknown
  Description of Unknown Bacteria <Prokaryote> sequence
  NT038
KQDGSADMDKKVKNGELVKTKVKLVSANGTNPVKISNVAEGTEDTDAVSFKQLKALQNKQVTLSASNAYANGGSDADVGKVTQTLSNGLNFKFKSTDGELLNIKADKDTVTITRASGANGAAATDADKIKVASDGISAGNKAVKNVAAGEISATSTDAINGSQLYAVAKGVTNLAGQVNKVGKRADAGTASALAASQLPQASMPGKSMVSIAGSSYQGQSGLAIGVSRISDNGKLIIRLSGTTNSQGKTGVAAGVGYQW

SEQ ID NO: 5 - protein, 521 aa, Unknown
  Description of Unknown Bacteria <Prokaryote> sequence
  NT067
VIVPEGTQLDEKQHIVINNGAEPQSFNPHKTEGVPESNVAYQLLEGLVTSDSEGKLQPGAAESWENTPDFKTWTFHLRKDAKWSNGDPVTAHDFVFAWRRLVDPATAAPYASYLSYLQVENAQDIIDGKKKPAELGVEAKDDYTFVVHATNPVPYAVSLTTHQSLLPLPQKVVEKLGDAWVKKENYVGNGAYKLANHIINEKIEFERNPLYWNDKETVINSATFLAIENPSTDVARYRAGDLDMTSYGLPPEQFAKLKKELLGEVYVTRTLGTYSYELNNKKAPFDNVNIRKALNLSLDRNVITDKVLGQGQTPTYVFTPTYIEEGHLIQQPAYSKEPMAQRNEEAIKLLEEAGYSKANPLKFSILYNTNENHKKVAIAAASMWKANTKGLIDVKLENQEWKTYIDSRRAGRYDVARAGWHADYNQATTFGNYFLSNSSNNTAKYANPEYDKAMAESYAATDAEGRAKAYAKAEEILGKDYGIVPIFNYVNPRLVKPYVKGYSGKDPQDHIYLRNLYIIKH

SEQ ID NO: 6 - protein, 252 aa, Unknown
  Description of Unknown Bacteria <Prokaryote> sequence
  NT001
KEDKKPEAAVAPLKIKVGVMSGPEHQVAEIAAKVAKEKYGLDVQFVEFNDYALPNEAVSKGDLDANAMQHKPYLDEDAKAKNLNNLVIVGNTFVYPLAGYSKKIKNVNELQDGAKVVVPNDPTNRGRALILLEKQGLIKLKDANNLLSTVLDIVENPKKLNITEVDTSVAARALDDVDLAVVNNTYAGQVGLNAQDDGVFVEDKDSPYVNIIVSRTDNKDSKAVQDFVKSYQTEEVYQEAQKHFKDGVVKGW

SEQ ID NO: 7 - protein, 243 aa, Unknown
  Description of Unknown Bacteria <Prokaryote> sequence
  NT016
SSGSKDVEQASVNELYTKGTTSLQEGSYSEAIRYLKATTERFPGSVYQEQAMLDLIYANYKTQDYTQVLLMVDSFLHQFPQSPNQAYAVYMAGLTNAATGDNFIQDFFGIDRATRETTSMRTAFSNFQNLVRVFPNSPYSQDALARMAYIKDALARHELEIAKFYAKRKAWVAVANRVVGMLKQYPDTKATYEGLFLMQAAYEKMGLTALANDTQKIIDANKDKTFAPIEKPNEPDLKVPAVK

SEQ ID NO: 8 - protein, 337 aa, Unknown
  Description of Unknown Bacteria <Prokaryote> sequence
  NT052
DTLEQQFQQGLEATKRGDYQTAFKLWLPLAEQGNASIQFNLGLMYKKGQGIKQDDFEAVKWYRKAAEQGVADAQLNLGNMYAKGLGVKQDDVEAVKWYRQAAEQGNAKAQFNLGLMYDNGRGVKQDYFEAVKWFRKAAEQGYADAQFNLGNMYYNGHGVKQDDFEAVKWYRKAAEQGYADAQFNLGNMYYNGHGVKQDDFEAVKWYRKAAEQGHAKAQYNLGNMYANGRGVKQDYFEAVKWYRKAAEQGYADAQANLGSAYSAGHGVRQDYIEAVKWFKKAAENGSADGQFKLGLVYLIGQGIQKDRTLAKEWLGKACDNGNQNGCEYYGELNRGER

SEQ ID NO: 9 - protein, 143 aa, Unknown
  Description of Unknown Bacteria <Prokaryote> sequence
  NT002
CSSFQNDDYAMNYKGQIGDPIMAIAMLSEQQHEWAGTPYVLGGVSRRGVDCSGFVQKTFFDRFNLRLPRSTVEQANYGKHVRKEHIQTGDLIFFKTGLGPNGYHVGIYVKEDKFLHASTRGGVVYSSMNNPYWSKAFWQVRRI

SEQ ID NO: 10 - protein, 146 aa, Unknown
  Description of Unknown Bacteria <Prokaryote> sequence
  NT026
VPLWKTDSPKTILAKEQHRLYLFLRQIQARAENSSEVWFLLINRNLATQQWCLTAQVKNNQTCDCLNPINCPKEVYAHFYYPYFPNKTMIQSHHIYPKEITRFDGIRNTIVTRCFILQAENERTLFLFFNVGSIRLKTNQFDSACN

SEQ ID NO: 11 - protein, 556 aa, Unknown
  Description of Unknown Bacteria <Prokaryote> sequence
  NT009
EQTVDIEVQGIRGFRAVRNTDLNVHLINKEEMDGSERYQHLVTKAVDRGLRVFGYYDSSVRFERKQRQGKRDLLIAHVTPGEPTKIAGTDVQIEGEAAQDENFNALRKNLPKDGVLVEHQTYDDYKTAISRLALNRGYFDGEFKISRLEISPETHQAWWRMLFDSGVRYHYGNITFSHSQIRDDYLNNILNIKSGDPYLMNNLSDLTSDFSSSNWFNSVLVQPNINHKSKTVDIEIILYPRKKNAMELGVGFDTDGGVHGQIGWTKPWINSRGHSLRSNLYLSAPKQTLEATYRIPLLKNPLNYYYDFAVGWEGEKENDTNTRALTLSALRYWNNARGWQYFGGLRARYDSFTQADITDKTLLLYPTVGFTRTRLRGGSFATWGDVQKITFDLSKRIWLSESSFIKVQASSAWIRTYAENHRIVARAEIGYLHTKDIEKIPPTLRFFAGGDRSVRGYGYKKIAPKNKNGKLVGGSRLLTGSLEYQYQVYPNWWAATFVDSGLVADNYTAKELRYGAGVGVRWASPVGAIKFDIATPIRDKDNSKNIQFYIGLGTEI

SEQ ID NO: 12 - protein, 126 aa, Unknown
  Description of Unknown Bacteria <Prokaryote> sequence
  NT025
IIAILATIAIPSYQNYTKKAAVSELLQASAPYKADVELCVYSTNETTNCTGGKNGIAADITTAKGYVKSVTTSNGAITVKGDGTLANMEYILQATGNAATGVTWTTTCKGTDASLFPANFCRSVTK

SEQ ID NO: 13 - protein, 162 aa, Unknown
  Description of Unknown Bacteria <Prokaryote> sequence
  NT028
PRTVSHQVISENDDIQLTGLINNLEKDNRTGIFHKVRTNRSSALMGDKALASVYNEWVGTRYRMGGTTKRGIDCSAFMQTTFSEVFGIELPRSTAEQRHLGRKINKSELKKGDLVFFRKNNHVGVYIGNNQFMHASTGQGVTISSLDEKYWARTYTQSRRIM

SEQ ID NO: 14 - protein, 895 aa, Unknown
  Description of Unknown Bacteria <Prokaryote> sequence
  NT029
STPDLPQNHKIITGTATVSHTENEMTIKQTTPTTQINWDSFNIGKDKEVKFEQPSTSAVAYNRVTGGNASHIQGKLTANGKVYLANPNGVIITKGAEINVAGLLATTKDLERISENGNTNTNKFTRKAKEGKVLTEGQVINEGEIKAKDFVVLNGDEVINKGNINVEKNSTINGEVYLSSSNNFTFTLSDSGISVALEDNTVQGIVKNEGIVKNEGSIKAGEITLSAKGRKEALDSLVVNNGVLEATKVSNRKGKIVLSADDVQLNNNSDIKGEIVNFGTEVTSNEDKKLKITSQTGSKVTSPKINFKGKSVNIKGDFGREDNTTYYDDEHKKLKTEVNIDVPNTENIQIADKDNAGTDSFIQTGALSSLLANNGKVNLKGKDVNISGNININSFRGTDSLLKLTNKGHININHADIHSKGRLFFITSLQNDVDFQSNITITDSKINLGNGAMGLGRSVNENDLDRWRRTEYSQRKKFNVNMRNVVFDQVDDVVVAGGFKEVNLNNIVATGQTNFYIDGGVSRNRNGVSSKYEYGVLDLDKRTQLSELDQRRRRWGYYPDLDLDMNKAYWHRFDMFASKNTGRSTIKDTEINISNSKINLKNGFVHLLAEKIKLDNSKIDITFDKDNSQDISTQINRLGMNGKVSMVNSHIKIVGDEKIDISAKAPYATMFLIGELIGEKSSIFVKSHQGYTFRTDGDTKIAGKNSKDDLKITAINTGGRTGKEVIINGAPGSIDNDANIANMAFTIGDNANTKTTIENADITALAPNGGTAYLSSKGVEIEVNPNSNFTFFELPREKNFNQTKINGDSTKLSERGFARLYDKINGVRASNLSAEQLNVTDSSEKIINTKLVSSLDVEKLVSVAVCDAGKGCEEQQFGDKGNNTKVSVGELEAEQ

SEQ ID NO: 15 - protein, 165 aa, Unknown
  Description of Unknown Bacteria <Prokaryote> sequence
  NT031
CIAPPKGLEKERFSINSYREISPQDLTCHCNTVRLGGKIVNTTVLANQTKIEVLSLPVSSISGKPFVELQSDGRFIVYFNGFVEPENLKERYITVGGQLTGTEKGKIEQADYTYPVVQADKYRIWTLSTTYNYPTDDWDEDDDWGFFRWRHRPWYVQPEIHYYLN

SEQ ID NO: 16 - protein, 117 aa, Unknown
  Description of Unknown Bacteria <Prokaryote> sequence
  NT015
AQNANVTTPQTQKMQVEKVDKALQKGEADRYLCQDDKVVRVVHATHKKYKKNLHYVTVTFQGVSEKLTLMISERGKNYANIRWMWQERDDFSTLKTNLGEILATQCVSQTSERLSGQ

SEQ ID NO: 17 - protein, 134 aa, Unknown
  Description of Unknown Bacteria <Prokaryote> sequence
  NT023
NTDIFSGDVYSASQAKEARSITYGTIVSVRPVKIQADNQGVVGTLGGGALGGIAGSTIGGGRGQAIAAVVGAIGGAIAGSKIEEKMSQVNGAELVIKKDDGQEIVVVQKADSSFVAGRRVRIVGGGSSLNVSVL

SEQ ID NO: 18 - protein, 315 aa, Unknown
  Description of Unknown Bacteria <Prokaryote> sequence
  NT100
IKKNENNNAYQFNHLKTLGLYIQNTTYFTDNFIITGGLRYEYFDQVVGRSTLKNIRSGYLAQKDGKLLYQLGSVYKFTPNIATFFNHAESFRPQNNRTLIINGELPAEQGKSFETGLKYENAYLNATVALFNINKRNVAETVNVNGTNELQIVGKQRSRGIEFDLNGQLTDNLSIAANYTYTKVKNLENHNNKLAVGKQLSGVPKHQASLFLAYNIGEFDFGNIRVGGGARYLGSWYAYNNTYTKAYKLPQAIVYDAFIAYDTKISGKKVSFQLNGKNLSNKVYSPSTSGNASRTLIPVALGYAREVILNTKIEF

SEQ ID NO: 19 - protein, 212 aa, Unknown
  Description of Unknown Bacteria <Prokaryote> sequence
  NT040
SISHFYVQIQTQNQQMLLHLKLQAELQRTLQLIGKDLRRLGFRALNAKLTESNLSLFELDEQGTAIFISQEDNAPPNSCVLFFYDLNKNGCIGKGSPKTCMKKGKNTSKSSTEELFGYKVSNKMIKTKLTYQSVIPTNCTAETCKRAFQQTACNAGGGWADLLDNNEYEITRLQFNWLIEGKGLEIKLKGNLKQTPNISYETSLVVALWNQK

SEQ ID NO: 20 - protein, 630 aa, Unknown
  Description of Unknown Bacteria <Prokaryote> sequence
  NT048
SGGGGSFDVDDVSNPSSSKPRYQDDTSSSRTKSKLENLSIPSLGGGMKLVAQNLRDRTKPSLLNEDDYMIFSSLSTIKADVEKENKHYTSPVGSIDEPSTTNPKENDHGQRYVYSGLYYIPSWNLNDLKNNKYYYSGYYGYAYYFGKQTATTLPVNGKVTYKGTWSFITAAENGKRYPLLSNGSQAYFRRSAIPEDIDLEVKNDENREKGLVSEFSADFGTKKLTGGLFYTKRQTHIQNHEKKKLYDIDAHIYSNRFRGKVNPTQKDSKEHPFTSEGTLEGGFYGPEGQELGGKFLAGDKKVFGVFSAKGTEENKKLPKETLIDGKLTTFSTKTTDAKTNATANATTSTAANTTTDTTANTITDAENFKTKDISSFGEADYLLIDNYPVPLLPESGDFISSKHHTVGKKTYQVKACCSNLSYVKFGMYYEVPPKEEEKDKEKKEKEKEKQATNLSNTYYQFLLGLRTPSSEIPKGGSAKYLGSWFGYLSDGSTSYSPSGDKKRENNALAEFNVNFADKTLKGQLKRHDNQNTVFTIDATFKSGKNNFTGTATANNVAIDPQSTQGTSNVNFTATVNGAFYGPNATELGGYFTYNGNPTDKSSSTVPSSSNSKNARAAVVFGARQQVETTK

SEQ ID NO: 21 - protein, 157 aa, Unknown
  Description of Unknown Bacteria <Prokaryote> sequence
  NT053
ADSSDKTWQLQTGQGLDAKRGQVNNQFTQVDTRLNRTDLRINRVGASATALASLKPAQLGEDDKFALSLGVGSYKNAQAMAMGAVFKPAENVLLNVAGSFSGSEKIVGAGVSWKFGSKSKPAVSTQSAVNSAEVLQLRQEISAMQKELAELKKALRK

SEQ ID NO: 22 - protein, 505 aa, Unknown
  Description of Unknown Bacteria <Prokaryote> sequence
  NT066
FKKSLIVAASFASLSLFNSATAELVYKPLEQPVEPAKPDLKIESVNEKFAEKYPNQYNSWRSTANGDGENIIYADEENPRLIVLWGGYAFAKEYNAPRGHFYAVTDVRNILRTGAPKTANDGPQAMACWTCKGPDVPRLIAEWGEKDYFNAKWAKGGPEIVNSIGCADCHDTTSKDFAEGKPALRIARPHILRALDALEKATAEKDKAEGRPHNNLSFNTAARTEKRAEICANCHVEYYFSGDIKQVTFPWDNGQTVDDIEKYYDDIGFTDWTHSLSKAPMLKAQHPDFEIWSLGMHGKNGVTCVDCHMPKVQGADGKVYTDHQIQNPFEAFDSTCANCHDQSKEKLRDIVTSRKKEVKDVMGRLEDQVVKAHFEAKEAWDAGATKKEMEAALMDIRHAQWRWDYTAASHGGHMHAPEVVLRVLASGLDKVADARTKLAVILTKHGVKTPVQIPDISTADKAWKVMGIDIEKERKAKEEFLKTVVPQWEQQAREKGLLVDPPAQK

SEQ ID NO: 23 - protein, 429 aa, Unknown
  Description of Unknown Bacteria <Prokaryote> sequence
  NT097
AAFQLAEVSTSGLGRAYAGEAAIADNASVVATNPALMSLFKTAQFSTGGVYIDSRINMNGDVNSYLNSGSMALTKYGSASQRNVVPGAFVPNLYFVAPVNDKFALGAGMNVNFGLKSEYDDSYDAGVFGGKTDLNAINLNLSGAYRVIEGLSLGLGVNAVYANAQVERNAGIIADSLQDSQVKGALKIVDSTNKAPDRLTSKDKSVVSLQDRAAWGFGWNAGVMYQFNEANRIGLAYHSKVDIDFADRTATSFGKKDIVAGKTGNLTFTLPDYLELSGFHQLTDKFAVHYSYKYTHWSRLTKLHASYENGEKAFDKELQYSNNSRVALGASYNLDEKLTLRAGIAYDQAASRHQRSAAIPDTDRTWYSLGGTYKFTPNLSVDLGYAYLKGKKVHFKEVQKAAGGHITTTANYTSQAHANLYGLNLNYSF

SEQ ID NO: 24 - protein, 422 aa, Unknown
  Description of Unknown Bacteria <Prokaryote> sequence
  P48 (NT007)
VNQVAILGEEYVGMRPSMKVREGDVVKKGQVLFEDKKNPGVIFTAPASGTITAINRGEKRVLQSVVINVEGDEKITFAKYSTEQLNTLSSEQVKQNLIESGLWTALRTRPFSKVPSIESEASSIFVNAMDTNPLAADPSVVLKEYSQDFTNGLTVLSRLFPSKPLHLCKAGDSNIPTADLENLQIHDFTGVHPAGLVGTHIHFIDPVGIQKTVWHINYQDVIAVGKLFTTGELYSERVISLAGPQVKEPRLVRTIIGANLSQLTQNELSAGKNRVISGSVLCGQIAKDSHDYLGRYALQVSVIAEGNEKEFFGWIMPQANKYSVTRTVLGHFSKKLFNFTTSENGGERAMVPIGSYERVMPLDILPTLLLRDLIVGDTDGAQELGCLELDEEDLALCSFVCPGKYEYGSILRQVLDKIEKEG

SEQ ID NO: 25 - protein, 437 aa, Unknown
  Description of Unknown Bacteria <Prokaryote> sequence
  HtrA (NT006)
TLPSFVSEQNSLAPMLEKVQPAVVTLSVEGKAKGDSRSPFLDDIPEEFKFFFGDRFAEQFGGRGESKRNFRGLGSGVIINASKGYVLTNNHVIDGADKITVQLQDGREFKAKLVGKDEQSDIALVQLEKSSNLTEIKFADSDKLRVGDFTVAIGNPFGLGQTVTSGVVSALGRSTGSDSGTYENYIQTDAAVNRGNSGGALVNLNGELIGINTAIISPSGGNAGIAFAIPSNQASNLVQQILEFGQVRRGLLGIKGGELNADLAKAFNVSAQQGAFVSEVLPKSAAEKAGLKAGDIITAMNGQKISSFAEMRAKIATTGAGKEISLTYLRDGKSHDVKVKLQADDGSQLSSKTELLALDGATLKDYDTKGVKGIEITKIQPNSLAAQRGLKAGDIIIGINRQMIENIRELNKVLETEPSAIALNILRGNSNFYLLVQ

SEQ ID NO: 26 - protein, 144 aa, Unknown
  Description of Unknown Bacteria <Prokaryote> sequence
  PE (NT035)
SAQIQKAEQNDVKLAPPTDVRSGYIRLVKNVNYYIDSESIWVDNQEPQIVHFDAVVNLDKGLYVYPEPKRYARSVRQYKILNCANYHLTQVRTDFYDEFWGQGLRAAPKKQKKHTLSLTPDTTLYNAAQIICANYGKAFSVDKK

SEQ ID NO: 27 - protein, 197 aa, Unknown
  Description of Unknown Bacteria <Prokaryote> sequence
  P26 (NT010)
MKNIAKVTALALGIALASGYASAEEKIAFINAGYIFQHHPDRQAVADKLDAEFKPVAEKLAASKKEVDDKIAAARKKVEAKVAALEKDAPRLRQADIQKRQQEINKLGAAEDAELQKLMQEQDKKVQEFQAQNEKRQAEERGKLLDSIQTATNNLAKAKGYTYVLDANSVVFAVEGKDITEEVLKSIPASEKAQEKK

SEQ ID NO: 28 - protein, 346 aa, Unknown
  Description of Unknown Bacteria <Prokaryote> sequence
  PHiD (NT080)
CSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPEFFTDVNQMYDALLNKSGATGVFTDFPDTGVEFLKGIK

SEQ ID NO: 29 - protein, 134 aa, Unknown
  Description of Unknown Bacteria <Prokaryote> sequence
  P6 (NT081)
CSSSNNDAAGNGAAQTFGGYSVADLQQRYNTVYFGFDKYDITGEYVQILDAHAAYLNATPAAKVLVEGNTDERGTPEYNIALGQRRADAVKGYLAGKGVDAGKLGTVSYGEEKPAVLGHDEAAYSKNRRAVLAY

SEQ ID NO: 30 - protein, 433 aa, Unknown
  Description of Unknown Bacteria <Prokaryote> sequence
  NT013
SEENPIFSTSDSGEYHELNTSPNKNSTALQPDEDATSYDDELQAKDDEVDEVKLSSDDLGTLPQHAQDALNGLLDAADQAIRITDQFSYTVTEGDTLKDVLVLSGLDDSSVQPLIKLDPELAHLKAGQQFYWILNKNDNLEYLNWLVSEKEERIYERLEDGKFKRQVIEKKSIWRKEVLKGEIQNSLNSSLREQGLDTRQISQLSNALQWQVSLRKLKKGTQFAILVSREYLGDKLTGQGNVEALRISSGGKNYYAVQAANGRYYNQQGETLGKGFARYPLQRQARVSSPFNPNRRHPVTGRVRPHKGVDFSVSQGTPVIAPADGTVEKVAYQAGGAGRYVMLRHGREYQTVYMHLSKSLVKAGQTVKKGERIALSGNTGISTGPHLHYEFRINGRAVNPLTVKLPGTSSGMTSAERKQFLVRVREAEKMLKP

SEQ ID NO: 31 - protein, 192 aa, Unknown
  Description of Unknown Bacteria <Prokaryote> sequence
  NT106
SSNPETLKATNDSFQKSETSIPHFSPLATGGVQLPKADDAYSLPNIEVKKGEDIDIRPPLIPLAIIQNSITKFDGERSLIVYPKQQAKLYNLQQVKRLLKDEGISSTTDGSILTTDWAKTERIGDKSIEIKYQIEQVMTADVSVLTVSILHMRRDGIIFTPNVSDKQYYTSERLNRIVLTLTTAYNKQLQDL

SEQ ID NO: 32 - protein, 537 aa, Unknown
  Description of Unknown Bacteria <Prokaryote> sequence
  NT107
QPDTGSLNRELEQRRIQPEAKPSGELFNQAAKSPYTAQYKQELKFPLTQVQILDRNNQEVVTDELAHILKNYVGKEVSLSDLSNLANEISEFYRNNNYLVAKAILPPQEIEQGTVKILLLKGNVGEIRLQNHSALSNKFVSRLSNTTVNTSEFILKDELEKFALTINDVPGVNAGLQLSAGKKVGEANLLIKINDAKRFSSYVSVDNQGNKYTGRYRLAAGTKVNNLTGWGDELKLDLLSSNQANLKNARIDYSSLIDGYSTRFGVTANYLHYKLGGNFKSLQSQGHSHNLGAYLLHPTIRTPNFRLSTKVSFNHQNLTDEQQAVTVKQKRKINSLTVGIDGSWNLIKDGTTYFSLSTLFGNLANQTNEKKHNAKEDFQPQSHFTVYNYRLSHEQILPKSFAFNIGINGQFADKTLESSQKMLLGGLSGVRGHQAGAASVDEGHLIQTEFKHYLPVFSQSVLVSSLFYDYGFGKYYKNSQSLAQSVKNSVKLQSVGAGLSFSDAGSYAINVSVAKPLDNNIDNADKHQFWLSMIKTF

SEQ ID NO: 33 - protein, 345 aa, Unknown
  Description of Unknown Bacteria <Prokaryote> sequence
  NT108
TSNIKNIQIPTTLNGSDPQQFGAKYTNRTYQQAALVPVSNIENQSAVINQGDFLTQLSNIKNYSSKLSTNFYDNYEKITNWVLSGANINELTQFNIHPQIMRGFDGYQNVLMTGYYSPILYARHTPQGQFKNPIYRMPVKKRLSRAQIYAGALTGKRLELAYSDSMLENFLLGVQGSGYVDFGDGNLNYFAYAGQNGYPYTAIGRLLVEDGEIPKEKMSIQAIREWGNRNPSRVQSLLERNEAYVFFKNDPSGKVKGSAGVPLVAMASVASDRNIIPSGSVLLVEVPDIDNNGNWIGTHKLHLMVALDVGGAVKGHHFDLYRGIGARAGHIAGLSKHYGRVWVLR

SEQ ID NO: 34 - protein, 534 aa, Unknown
  Description of Unknown Bacteria <Prokaryote> sequence
  NT109
SSNKYDAEAINISGSLRMQSYRLLYEMQEQPESVETNLRRYHISLHSSALLEVQNQFFTPNVLKHSYQNILQRWTNMEKYARQQDVKNYSKQLTNYVADVDYFVFELQRFSEQKWILGVSVLGFAMLLILLMVSYVIWYTNREVVKPLHLMTKASMQVQMRQFNHIPLDTRKQNELGTLARVFTQMSTELGQLYSRLEEAVNEKTQKLRQTNRTLSTLYQSAQLLNTNTINDKILNQVLHYIFISDHLNFVKVEVMGAEHWDITLGKQDANNELQIETLSVDNEELGVLSWQAGLPCPDPRIMQNLAQMLARALYFHKNLRQKEQILLMEERSIIARELHDSLAQVLSFLQIQLTLLKHNLKKEDEQSKEKSLAIIANFEQALSGGYAQLRELLATFRLTIQEANLQLALKQVIDSLRSQTTMQMNVNCQLPSQSLNPQQLVHVLQIVREATTNAIKHSQGTVIEISARINAEGEYEILVEDDGVGIPNLEEPEGHYGLNIMAERCRQLNAQLHIHRREQGGTQVKITLPHTLY

SEQ ID NO: 35 - protein, 402 aa, Unknown
  Description of Unknown Bacteria <Prokaryote> sequence
  NT110
SSRKLKLQSLANKGDVRALQVLKLQEHPGRFITVVQILLNMVAILGGGIGESALSPYIADILNRSFEGSWIEPTASTIAFILVTCLFILFADLIPKRIAITYPEMVALSVVGIMNFSMYVFKPLVWFFDTIANVFFRLFRISTVREDGMTSEDIFAVVEAGAEAGVLKTQEHYLIENIFDMQARTVTSTMTTRENIVYLDRTFSRQEVMDTLSRDSHSKIVICDNGLDKILGYIESHTLLTMYLQNENVVLTDPKLLRKALFVPDTLSLYEVLELFKSTGEDFAIIVNEYALVVGIVTLNDVMSIVMGELVSNEEEYIVSRDENSWLIDGATPLKEVTRVLDIAYFPDEENYETISGFMMYMLRKIPKKTDSVVYGKYKFEVIDTENFKIDQILVSLVKEQE

SEQ ID NO: 36 - protein, 265 aa, Unknown
  Description of Unknown Bacteria <Prokaryote> sequence
  NT111
QDKDTEAKIKQLNQTVAQLSAENTKLKEQIEKTVPAIIVENDEIFNQSEIIKHPKSKEDYQPEETKIEYSISTIKTNIDWLNDLLWKKLTENEETKNISREQFVARYQTAFEEDKKEAKETPSFGISHSIWTNFIGQKEKLATFAISFYDYEGGAHGIEGNRYFTIDLTTRHILTLNDLFNEKDLPKVKTLLWEQYNNSNKEYEPIIEADSFNLSNNIYLDSKGVHFIYDVYEIAPYAAGEQDLMLHFGQLEELFKPEFRQGNDI

SEQ ID NO: 37 - protein, 231 aa, Unknown
  Description of Unknown Bacteria <Prokaryote> sequence
  NT112
ANHNATKQASERNDSLEDFNRTMWKFNYNVIDRYVLEPAAKGWNNYVPKPISSGLAGIANNLDEPVSFINRLIEGEPKKAFVHFNRFWINTVFGLGGFIDFASASKELRIDNQRGFGETLGSYGVDAGTYIVLPIYNATTPRQLTGAVVDAAYMYPFWQWVGGPWALVKYGVQAVDARAKNLNNAELLRQAQDPYITFREAYYQNLQFKVNDGKLVESKESLPDDILKEID

SEQ ID NO: 38 - protein, 341 aa, Unknown
  Description of Unknown Bacteria <Prokaryote> sequence
  NT113
SNSSNQGINYDEAFAKDTQGLDILTGQFSHNIDRIWGVNELLVASRKDYVKYTDSFYTRSHVSFDEGNIVIETQQDLNRLHNAIVHTLLMGADAKGIDLFTSGDVPISSRPFLLGQVVDHQGQQIANQVIASNFATYLIQNKLQTRRLQNGHTVQFVSVPMIANHVEVRARKYLPLIRKAAQRYGIDESLILGIMQTESSFNPYAISYANAIGLMQVVPHTAGRDVFAMKGKGGQPSTRYLYDPANNIDAGVSYLWILQNQYLDGITNPTSKRFAMISAYNSGAGAVLRVFDNDKDTAIYKINQMYPEQVYRILTTVHPSSQARNYLLKVDKAQKKFRVRR

SEQ ID NO: 39 - protein, 574 aa, Unknown
  Description of Unknown Bacteria <Prokaryote> sequence
  NT114
DSPNTATASINLEQEKQNWASIQHQDYLKRLKQREVFLQVEGLLKSAVKKQQFSEAIQNITKTLIDSLQGYPLQYDLLARFWETKIAFLQNDDIQGKQQAINEVNALVQQNYPFVTPAFQALLQKLSTLNEQQTSATSDNAKENNRVQKEQNQVENPKQLAEIVRKSDPNTLDKTVLIDAFPRYLKTLPEQMNNLSFESYQKWANTWQLSEDEIKQWKIAFLNRFFDNENTDFQKWRDEQIRQLQTDNLTERRLRMAIWQKTELTSWLNLLSAESKSKQEWRYWEAKQDILKNTKKLTALSKERGFYPMLAATQLKQAYQLNVPIASSFTQAEQLPFKQVFAMITELRELGRNGLAKQRWRILLDNVDFTTQLKLSEYAKNQQWFELAVDASIVAKAWGYLSLRLPNAYSEYFNAALQNLNISKTFAMAIARQESAWNPMAQSSANARGLMQLLPSTAKLTAENNQLPYQGEQDLFKPLNNILLGTAHLNELNGKYPNNRILIAAAYNAGANRVEKWLSRASGKLALDEFVASIPFYETRGYVQNVVAYDFYYQILQNKENPQIFSQEELNRLY

SEQ ID NO: 40 - protein, 147 aa, Unknown
  Description of Unknown Bacteria <Prokaryote> sequence
  NT115
GWHFQQSVTMPNEWRTLALESDDSYNDFTVIMRRKLQENQVNVVNLEQNIPILRINKQITSDQVASIFKHGREAEKLLMLEVEATFRLANGESYPINAKVNRTFFDNARAALAKSEEREVIWNDMREQVARQLIVKIIALQNQIKRK

SEQ ID NO: 41 - protein, 612 aa, Unknown
  Description of Unknown Bacteria <Prokaryote> sequence
  NT116
SGGGGSFDVDDVSNPSSSKPRYQDDTSSSRTKSNLEKLSIPSLGGGMKLVAQNLSGNKEPSFLNENGYISYFSSPSTIEDDVKNVKTENKIHTNPIGLEPNRALQDPNLQKYVYSGLYYIENWKDFSKLATEKKAYSGHYGYAFYYGNKTATDLPVSGVATYKGTWDFITATKYGQNYSLFSNARGQAYFRRSATRGDIDLENNSKNGDIGLISEFSADFGTKKLTGQLSYTKRKTDIQQYEKEKLYDIDAHIYSNRFRGKVTPTKSTSDEHPFTSEGTLEGGFYGPNAEELGGKFLARDKRVFGVFSAKETPETEKEKLSKETLIDGKLITFSTKTADATTSTTASTTADVKTDEKNFTTKDISSFGEADYLLIDNYPVPLFPEGDTDDFVTSKHHDIGNKTYKVEACCKNLSYVKFGMYYEDKEKKNTNQTGQYHQFLLGLRTPSSQIPVTGNVKYLGSWFGYIGDDKTSYSTTGNKQQDKNAPAEFDVNFDNKTLTGKLKRADSQNTVFNIEATFKNGSNAFEGKATANVVIDPKNTQATSKVNFTTTVNGAFYGPHATELGGYFTYNGNNPTATNSESSSTVPSPPNSPNARAAVVFGAKRQVEKTNK

SEQ ID NO: 42 - protein, 350 aa, Unknown
  Description of Unknown Bacteria <Prokaryote> sequence
  NT117
NTIIPNYNTDAHLYEFTQTYDLVVPKGSQGQTNLWVPLPFNGEYQQVKSIHFEGNYMNAYVTENNKYGAKTLFATWDKDAQKRDLKVTMVIETKDREPMVKGALENYTPPKDIQYSVDVQEYLKATPHIKTDGIVKEFADKILGKETNPLKKAELIHHWIVKNMERDNSVLGCGDGDVEKILTTGVLKGKCTDINSVFVALARAAGIPAREIFGIRLGTAEKMGKYSKGAFGSANEQGIVNVSGGQHCRAEFYLAGFGWVPVDSADVAKMRLAEKKSVEDKDTQAVAKYLFGNWEANWVGFNHARDFDLYPQPELAPINNFGYPYAEVGGDPLNSFDPKEFKYDYVSKKL

SEQ ID NO: 43 - protein, 184 aa, Unknown
  Description of Unknown Bacteria <Prokaryote> sequence
  NT118
WFYSLNQETADLSELVKKPDSPDYVGYKMETTVFSPEGKKQYLALSDKIEHYTVNEQTLFTAPLVYLYPTTSNEKEQNPNQNVDFFSTQNWKLSANQARLTKDQILYLEGNVVVQSLTSDSRLQRIETESAVVNLKTQDMTSETQVKIKGKNFSSTGLKLVGNLRQQVATLKEQVKTYYEVSKQ

SEQ ID NO: 44 - protein, 909 aa, Unknown
  Description of Unknown Bacteria <Prokaryote> sequence
  NT123
QSLELSPNNDLPFDPNIQHGKLSNGLQYFVLKNTEPKERVYIRLVINAGSMHEDDDQKGIAHLVEHMAFNGSKKYPENQIINALEKLGMKFARDINAFTDFENTVYTLNLDSNNQQKLELAFDVINEWMNNITFLPKDVDGERGVVQEEWRRRLSPMLRIGNKKSAIEMAGSRYVLRDPIGDMDIIKTISAKRVADFYHKWYRPDNMSVIIVGDIDTKQVVKLLKQNLSQENPITKTTLEKIDFNIPLINKWRLDSISEQGTTIPSIELSFFENTIETNTLASYKQELIQQITTRLLNLRLQQWEKETENGVDSANFYRTHLGKETLQSIFSLQLIDTQYSKTIDKLFAFIASIKQQGFTQNELNGEIKRLTQLNEKQLNIRSGSLKIADDLITSVANKQVVLSVNDRYELNKRFLSQITLADLQRTLNQTLALKAKLLLITQPLPQKALPFDVVEIETRWNNAMKMQQHQWDEKKQIEKLPHLTFNTGSLSQEKYWDRGDIYEFRLSNGSKLIYHYSDKTPNQVHFRAVTQGGLRSIPDKDYHLLRAAVSVVDETGVGELSLSAVNQIFSRDPLVIATVIDDDKQGFTGVSKPKDLENLLTLFRLKLRSSPISDLALEKYRRETRDYFKQIDLETQFMQAVSKLRFPNIETVYTQKQAQQLAFDKNQLSNAYQRYILNKTDFTYFIIGDIELNQVKKLAERYLASVESKTQIRHFVPTIIHTPTQSFIMNGLKEPRADVEIYLTADNTWRAEQKYLFNILADIVQEKLRLILREKVSGVYSVNSWFMQDVHTPQIEGKIEFSCDPKRVEELTHLTNQVLDDIVKNGIDENLLRKKLAEQHTQIRREFDSLVSVASIIEESYWQQDNPDAIYTYQHLDQLATKATIDALAQKALKKSGRFVSVLKAATY

SEQ ID NO: 45 - protein, 458 aa, Unknown
  Description of Unknown Bacteria <Prokaryote> sequence
  NT124
WFTGKKAEEEYLHQLKQLNQLFTKTEALEESKIFYKNIKFERGLFASHIQDQIEIHKANETIIIPLSSTLYHGPLPLDRVAKLNFVPAIFSSQTLLGKNATTQAFFDITESEKPLQLNFAMNYSLSGNAELKLASGQYHNEQSKTDFDWSNVVLNIDLNQNTPNNYVLSVDTFNSNAPNHAVSTASSIKIKDLVVQGSLQSTKWPFIYSGNINSKIGYFEQNTESPETGEKFSLIQKNSQANLTTQVEGDTVNIINKTNLDELHINGNNLGKVTNNVEFNHIDGNALQELLNILVAIGKADSDMPLSKTLVQKLQQAGMIIANNQPQIKFTPLSISDEKGKVALDLNIALVPNPKFDLMRSGLYKQFKDFSINFDVNKETAISLLSKFVPENQKQDFVHRLNELVTEGEVNDIIVNTDKTVTLTLALENNDLKLNGKPIPEEQLKVVLFILVMGGFGR

SEQ ID NO: 46 - protein, 6 aa, Unknown
  Description of Unknown Bacteria <Prokaryote> sequence
  Linker 1
GSGGGG

SEQ ID NO: 47 - protein, 8 aa, Unknown
  Description of Unknown Bacteria <Prokaryote> sequence
  Linker 2
GSGSGGGG

SEQ ID NO: 48 - protein, 17 aa, Unknown
  Description of Unknown Bacteria <Prokaryote> sequence
  Histidine Tag
MGSSHHHHHHENLYFQG

SEQ ID NO: 49 - protein, 277 aa, Unknown
  Description of Unknown Bacteria <Prokaryote> sequence
  NT018 Variant from Fi176
KHGQKRDDLNKALYFSRLEEIEQDNSQGLVENVEQLKQELQKTLLDDVPSKVQENVDYSGKSYGKIWFVSGVLALGIIAGSPYFMVGSWQAESMLEQTYAKLPYFFDRMKDEDKNPFSDTEMQQFSTALRIDLQKNPTDAKKWWMLGQIGMNLGDARLAFDSYQKANKLEPDNVQYKLGYARILMFSEDATDKLKGGNLLREVIRQEHTNIEALSLLAFRYFETEDYKMAAVTWAMMLRLMPKDDERVPLIEKSIRTARDALEAQNEEKSKSITPEK

SEQ ID NO: 50 - protein, 160 aa, Unknown; identical to SEQ ID NO: 2
  Description of Unknown Bacteria <Prokaryote> sequence
  NT024Variant from Fi176

SEQ ID NO: 51 - protein, 102 aa, Unknown; identical to SEQ ID NO: 3
  Description of Unknown Bacteria <Prokaryote> sequence
  NT032 Variant from Fi176

SEQ ID NO: 52 - protein, 521 aa, Unknown
  Description of Unknown Bacteria <Prokaryote> sequence
  NT067 Variant from Fi176
VIVPEGTQLDEKQHIVFNNGAEPQSFDPHKTEGVPESNVAYQLLEGLVTSDSEGKLQPGVAESWENTPDFKTWTFHLRKDAKWSNGDPVTAHDFVFAWRRLVDPATAAPYASYLSYLQVENAQDIIDGKKKPAELGVEAKDDYTFVVHAINPVPYAVSLTTHQSLLPLPQKVVEKLGDAWVKKENYVGNGAYKLANHIINEKIEFERNPLYWNDKETVINSATFLAIENPSTDVARYRAGDLDMTSYGLPPEQFAKLKKELLGEVYVTRTLGTYSYELNNKKAPFDNVNIRKALNLSLDRHVITDKVLGQGQTPTYVFTPTYIEEGHLIQQPAYSKEPMAQRNEEAIKLLEEAGYSKANPLKFSILYNTNENHKKVAIAAASMWKANTKGLIDVKLENQEWKTYIDSRRAGRYDAARAGWSADYNQATTFGNYFLSNSSNNTAKYANPEYDKAMAESYAATDAEGRAKAYAKAEEILAKDYGIVPIFNYVNPRLVKPYVKGYSGKDPQDHIYLRNLYIIKH

SEQ ID NO: 53 - protein, 157 aa, Unknown
  Description of Unknown Bacteria <Prokaryote> sequence
  NT038 Variant from R2846
KQDGSADMDKKVKNGELVKTKVKLVSANGTNPVKISNVAEGTEDTDAVSFKQLKALQNKQVTLSASNAYANGGSDADVGKVTQTLSNGLNFKFKSTDGELLNIKADKDTVTITRASGANGAAATDADKIKVASDGISAGNKAVKNVAAGEISATSTD

SEQ ID NO: 54 - protein, 252 aa, Unknown
  Description of Unknown Bacteria <Prokaryote> sequence
  NT001 Variant from Fi176
KEDKKPEAAAAPLKIKVGVMSGPEHQVAEIAAKVAKEKYGLDVQFVEFNDYALPNEAVSKGDLDANAMQHKPYLDEDAKAKNLNNLVIVGNTFVYPLAGYSKKIKNVNELQEGAKVVVPNDPTNRGRALILLEKQGLIKLKDANNLLSTVLDIVENPKKLNITEVDTSVAARTLDDVDLAVVNNTYAGQVGLNAQDDGVFVEDKDSPYVNIIVSRTDNKDSKAVQDFVKSYQTEEVYQEAQKHFKDGVVKGW

SEQ ID NO: 55 - protein, 243 aa, Unknown
  Description of Unknown Bacteria <Prokaryote> sequence
  NT016 Variant from Fi176
SSGSKDVEQASVNELYTKGTTSLQEGSYSEAIRYLKATTERFPGSVYQEQAMLDLIYANYKTQDYTQVLLMVDSFLHQFPQSPNQAYAVYMAGLTNAATGDNFIQDFFGIDRATRETTSMRTAFSNFQNLVRVFPNSPYSQDALARMAYIKDALARHELEIAKFYAKRKAWVAVANRVVGMLKQYPDTKATYEGLFLMQEAYEKMGLTALANDTQKIIDANKDKTFAPIEKPNEPDLKVPAVK

SEQ ID NO: 56 - protein, 337 aa, Unknown; identical to SEQ ID NO: 8
  Description of Unknown Bacteria <Prokaryote> sequence
  NT052 Variant from R2846

SEQ ID NO: 57 - protein, 143 aa, Unknown
  Description of Unknown Bacteria <Prokaryote> sequence
  NT002 Variant from Fi176
CSSFQNDDYAMNYKGQIGDPIMAIAMLSEQQHEWAGTPYVLGGVSRRGVDCSGFVQKTFFDRFNLRLPRSTVEQANYGKHVRKEDIQTGDLIFFKTGRGPNGYHVGIYVKEDKFLHASTRGGVVYSSMNNPYWSKAFWQVRRI

SEQ ID NO: 58 - protein, 146 aa, Unknown
  Description of Unknown Bacteria <Prokaryote> sequence
  NT026 Variant from FI176
VPLWKTDSPKTILAKEQHRLYLFLRQIQARAENSSEVWFLLINRNLATQQWCLTAQVKNNQTCDCLNPINCPKEVYVHFYYPYFPNKTIIQSHHIYPKEITRFDGIRNTIVTRCFILQAENERTLFLFFNVGSIRLKTNQFDSACN

SEQ ID NO: 59 - protein, 556 aa, Unknown
  Description of Unknown Bacteria <Prokaryote> sequence
  NT009Variant from Fi176
EQTVDIEVQGIRGFRAVRNTDLNVNLINKEEMDGSERYQHLVTKAVDRGLRVFGYYDSSVRFERKQRQGKRDLLIAHVTPGEPTKIAGTDVQIEGEAAQDENFNALRKNLPKDGVLVEHQTYDDYKTAISRLALNRGYFDGEFKISRLEISPETHQAWWRMLFDSGVRYHYGNITFSHSQIRDDYLNNILNIKSGDPYLMNNLSDLTSDFSSSNWFSSVLVQPNINHKSKTVDIEIILYPRKKNAMELGVGFDTDGGVHGQIGWTKPWINSRGHSLRSNLYLSAPKQTLEATYRIPLLKNPLNYYYDFAVGWEGEKENDTNTRALTLSALRYWNNAHGWQYFGGLRTRYDSFTQADITDKTLLLYPTVGFTRTRLRGGSFATWGDVQKITFDLSKRIWLSESSFIKVQASSAWIRTYAENHRIVARAEIGYLHTKDIEKIPPTLRFFAGGDRSVRGYGYKKIAPKNRNGKLVGGSRLLTTSLEYQYQVYPNWWAATFADSGLAADNYTAKELRYGAGVGVRWASPVGAIKFDIATPIRDKDNSKNIQFYIGLGTEI

SEQ ID NO: 60 - protein, 126 aa, Unknown; identical to SEQ ID NO: 12
  Description of Unknown Bacteria <Prokaryote> sequence
  NT025Variant from Fi176

SEQ ID NO: 61 - protein, 162 aa, Unknown; identical to SEQ ID NO: 13
  Description of Unknown Bacteria <Prokaryote> sequence
  NT028Variant from Fi176

SEQ ID NO: 62 - protein, 895 aa, Unknown; identical to SEQ ID NO: 14
  Description of Unknown Bacteria <Prokaryote> sequence
  NT029 Variant from R2846

SEQ ID NO: 63 - protein, 183 aa, Unknown
  Description of Unknown Bacteria <Prokaryote> sequence
  NT031Variant from R2846
MKGKITLFFTALCFGLTGCIAPPKGLEKERFSINSYREISPQDLTCHCNTVRLGGKIVNTTVLANQTKIEVLSLPVSSISGKPFVELQSDGRFIVYFNGFVEPENLKERYITVGGQLTGTEKGKIEQADYTYPVVQADKYRIWTLSTTYNYPTDDWDEDDDWGFFRWRHRPWYVQPEIHYYLN

SEQ ID NO: 64 - protein, 117 aa, Unknown
  Description of Unknown Bacteria <Prokaryote> sequence
  NT015Variant from Fi176
AQNANVTTPQAQKMQVEKVDKALQKGEADRYLCQDDKVVRVVHATHKKYKKNLHYVTVTFQGVSEKLTLMISERGKNYANIRWMWQERDDFSTLKTNLGEILATQCVSQTSERLSGQ

SEQ ID NO: 65 - protein, 134 aa, Unknown
  Description of Unknown Bacteria <Prokaryote> sequence
  NT023Variant from Fi176
NTDIFSGDVYSASQAKEARSITYGTIVSVRPVKIQADNQGVVGTLGGGALGGIAGSAIGGGRGQAIAAVVGAIGGAIAGSKIEEKMSQVNGAELVIKKDDGQEIVVVQKADSSFVAGRRVRIVGGGSSLNVSVL

SEQ ID NO: 66 - protein, 345 aa, Unknown
  Description of Unknown Bacteria <Prokaryote> sequence
  NT100 Variant from R2846
MDLGPIYNTRDINDGKVINIDNPNYTNPVAIKKNENNNAYQFNHLKTLGLYIQNTTYFTDNFIITGGLRYEYFDQVVGRSTLKNIRSGYLAQKDGKLLYQLGSVYKFTPNIATFFNHAESFRPQNNRTLIINGELPAEQGKSFETGLKYENAYLNATVALFNINKRNVAETVNVNGTNELQIVGKQRSRGIEFDLNGQLTDNLSIAANYTYTKVKNLENHNNKLAVGKQLSGVPKHQASLFLAYNIGEFDFGNIRVGGGARYLGSWYAYNNTYTKAYKLPQAIVYDAFIAYDTKISGKKVSFQLNGKNLSNKVYSPSTSGNASRTLIPVALGYAREVILNTKIEF

SEQ ID NO: 67 - protein, 505 aa, Unknown
  Description of Unknown Bacteria <Prokaryote> sequence
  NT066 variant from Fi176
FKKSLIVAASFASLSLFNSATAELVYKPLEQPVEPAKPDLKIESVNEKFAEKYPNQYNSWRSTANGDGENIIYADEENPRLIVLWGGYAFAKEYNAPRGHFYAVTDVRNILRTGAPKTANDGPQAMACWTCKGPDVPRLIAEWGEKDYFNAKWAKGGPEIVNSIGCADCHDTTSKDFAEGKPALRIARPHILRALDALEKATAEKDKAEGRPHNNLSFNTAARTEKRAEICANCHVEYYFAGDIKQVTFPWDNGQTVDDIEKYYDDIGFTDWTHSLSKAPMLKAQHPDFEIWSLGMHGKNGVTCVDCHMPKVQGADGKVYTDHQIQNPFEAFDHTCANCHDQSKEKLRDIVTSRKKEVKDVMGRLEDQVVKAHFEAKAAWDAGATKEEMEAALMDIRHAQWRWDYTAASHGGHMHAPEVVLRVLASGLDKVADARTKLAVILTKHGVKTPVQIPDISTADKAWKVMGIDIEKERKAKEEFLKTVVPQWEQQAREKGLLVDPPAQK

SEQ ID NO: 68 - protein, 6 aa, Unknown
  Description of Unknown Bacteria <Prokaryote> sequence
  His tag
HHHHHH

SEQ ID NO: 69 - protein, 28 aa, Unknown
  Description of Unknown Bacteria <Prokaryote> sequence
  NT018 N-terminus
MNFTLIFILTTLVVALICFYPLLRQFKA

SEQ ID NO: 70 - protein, 20 aa, Unknown
  Description of Unknown Bacteria <Prokaryote> sequence
  NT024 N-Terminus
MKLKLFFHIVLLCFSLPVWA

SEQ ID NO: 71 - protein, 19 aa, Unknown
  Description of Unknown Bacteria <Prokaryote> sequence
  NT032 N-terminus
MKKFALATIFALATTSAFA

SEQ ID NO: 72 - protein, 20 aa, Unknown
  Description of Unknown Bacteria <Prokaryote> sequence
  NT067 N terminus
MQHKLLFSAIALALSYSVQA

SEQ ID NO: 73 - protein, 23 aa, Unknown
  Description of Unknown Bacteria <Prokaryote> sequence
  NT038 N-terminus
MPFQYVTEDGKTVVKVGNGYYEA

SEQ ID NO: 74 - protein, 21 aa, Unknown
  Description of Unknown Bacteria <Prokaryote> sequence
  NT001 N-terminus
MKLKQLFAITAIASALVLTGC

SEQ ID NO: 75 - protein, 19 aa, Unknown
  Description of Unknown Bacteria <Prokaryote> sequence
  NT016 N-terminus
MRKIKSLALLAVAALVIGC

SEQ ID NO: 76 - protein, 11 aa, Unknown
  Description of Unknown Bacteria <Prokaryote> sequence
  NT052 N-terminus
MLLFILSIAWA

SEQ ID NO: 77 - protein, 18 aa, Unknown
  Description of Unknown Bacteria <Prokaryote> sequence
  NT002 N-terminus
MKVYKSFLIATASLFLFA

SEQ ID NO: 78 - protein, 24 aa, Unknown
  Description of Unknown Bacteria <Prokaryote> sequence
  NT026 N-terminus
MQKGMTLVELLIGLAIISIVLNFA

SEQ ID NO: 79 - protein, 22 aa, Unknown
  Description of Unknown Bacteria <Prokaryote> sequence
  NT009 N-terminus
MNKTLLKLTALFLALNCFPAFA

SEQ ID NO: 80 - protein, 23 aa, Unknown
  Description of Unknown Bacteria <Prokaryote> sequence
  NT025 N-terminus
MKLTTQQTLKKGFTLIELMIVIA

SEQ ID NO: 81 - protein, 21 aa, Unknown
  Description of Unknown Bacteria <Prokaryote> sequence
  NT028 N-terminus
MLKRILVIIGLAVLATACSNA

SEQ ID NO: 82 - protein, 21 aa, Unknown
  Description of Unknown Bacteria <Prokaryote> sequence
  NT029 N-terminus
MYKLNVISLIILTTYTGATYA

SEQ ID NO: 83 - protein, 18 aa, Unknown
  Description of Unknown Bacteria <Prokaryote> sequence
  NT031 N-terminus
MKGKITLFFTALCFGLTG

SEQ ID NO: 84 - protein, 17 aa, Unknown
  Description of Unknown Bacteria <Prokaryote> sequence
  NT015 N-terminus
MLKKTSLIFTALLLAGC

SEQ ID NO: 85 - protein, 20 aa, Unknown
  Description of Unknown Bacteria <Prokaryote> sequence
  NT023 N-terminus
MKKTNMALALLVAFSVTGCA

SEQ ID NO: 86 - protein, 30 aa, Unknown
  Description of Unknown Bacteria <Prokaryote> sequence
  NT100 N-terminus
MDLGPIYNTRDINDGKVINIDNPNYTNPVA

SEQ ID NO: 87 - protein, 26 aa, Unknown
  Description of Unknown Bacteria <Prokaryote> sequence
  NT040 N-terminus
MMKTLLKGQTLLALMISLTLSSLLLL

SEQ ID NO: 88 - protein, 18 aa, Unknown
  Description of Unknown Bacteria <Prokaryote> sequence
  NT048 N-terminus
MKSVPLITGGLSFLLSAC

SEQ ID NO: 89 - protein, 22 aa, Unknown
  Description of Unknown Bacteria <Prokaryote> sequence
  NT124 N-terminus
MKKSKIAAGVVISLAAVWCAGA

SEQ ID NO: 90 - protein, 33 aa, Unknown
  Description of Unknown Bacteria <Prokaryote> sequence
  NT066 N-terminus
MKIYLRFVWILIIILNFLLNLFITTNGVIIVNA

SEQ ID NO: 91 - protein, 22 aa, Unknown
  Description of Unknown Bacteria <Prokaryote> sequence
  NT097 N-terminus
MKKFNQSILATAMLLAAGGANA

SEQ ID NO: 92 - protein, 25 aa, Unknown
  Description of Unknown Bacteria <Prokaryote> sequence
  P48 N-terminus
MITIKKGLDLPIAGKPAQVIHSGNA

SEQ ID NO: 93 - protein, 26 aa, Unknown
  Description of Unknown Bacteria <Prokaryote> sequence
  HtrA N-terminus
MKKTRFVLNSIALGLSVLSTSFVAQA

SEQ ID NO: 94 - protein, 16 aa, Unknown
  Description of Unknown Bacteria <Prokaryote> sequence
  PE N-terminus
MKKIILTLSLGLLTAC

SEQ ID NO: 95 - protein, 18 aa, Unknown
  Description of Unknown Bacteria <Prokaryote> sequence
  PhiD N-terminus
MKLKTLALSLLAAGVLAG

SEQ ID NO: 96 - protein, 19 aa, Unknown
  Description of Unknown Bacteria <Prokaryote> sequence
  P6 N-terminus
MNKFVKSLLVAGSVAALAA

SEQ ID NO: 97 - protein, 42 aa, Unknown
  Description of Unknown Bacteria <Prokaryote> sequence
  NT013 N-terminus
MPVQHVKLARDRRKKRTYIKVGVFFVAILLILTGILLTIKDK

SEQ ID NO: 98 - protein, 17 aa, Unknown
  Description of Unknown Bacteria <Prokaryote> sequence
  NT106 N-terminus
MKKIILNLVTAIILAGC

SEQ ID NO: 99 - protein, 28 aa, Unknown
  Description of Unknown Bacteria <Prokaryote> sequence
  NT107 N-terminus
MKMRPRYSVIASAVSLGFVLSKSVMALG

SEQ ID NO: 100 - protein, 24 aa, Unknown
  Description of Unknown Bacteria <Prokaryote> sequence
  NT108 N-terminus
MSVCKPFWFKTFSISIITALLVSC

SEQ ID NO: 101 - protein, 30 aa, Unknown
  Description of Unknown Bacteria <Prokaryote> sequence
  NT109 N-terminus
MIMELFHTILAIVALILSSAVVSSAEISLA

SEQ ID NO: 102 - protein, 30 aa, Unknown; identical to SEQ ID NO: 101
  Description of Unknown Bacteria <Prokaryote> sequence
  NT110 N-terminus

SEQ ID NO: 103 - protein, 19 aa, Unknown
  Description of Unknown Bacteria <Prokaryote> sequence
  NT111 N-terminus
MKKTLVAALISSVILLTGC

SEQ ID NO: 104 - protein, 19 aa, Unknown
  Description of Unknown Bacteria <Prokaryote> sequence
  NT112 N-terminus
MKTKVILTALLSAIALTGC

SEQ ID NO: 105 - protein, 16 aa, Unknown
  Description of Unknown Bacteria <Prokaryote> sequence
  NT113 N-terminus
MKKYLLLALLPFLYAC

SEQ ID NO: 106 - protein, 19 aa, Unknown
  Description of Unknown Bacteria <Prokaryote> sequence
  NT114 N-terminus
MKKVALISLCIFTALSAFA

SEQ ID NO: 107 - protein, 35 aa, Unknown
  Description of Unknown Bacteria <Prokaryote> sequence
  NT115 N-terminus
MKYLHFTRPTIKVIFMINSIKTLLLIATLAILSAC

SEQ ID NO: 108 - protein, 18 aa, Unknown
  Description of Unknown Bacteria <Prokaryote> sequence
  NT116 N-terminus
MKSVPLITGGLSFLLSAC

SEQ ID NO: 109 - protein, 19 aa, Unknown
  Description of Unknown Bacteria <Prokaryote> sequence
  NT117 N-terminus
MKKLIAVAVFSACGSLAHA

SEQ ID NO: 110 - protein, 18 aa, Unknown
  Description of Unknown Bacteria <Prokaryote> sequence
  NT118 N-terminus
MNIRWNVILGVIALCALA

SEQ ID NO: 111 - protein, 17 aa, Unknown
  Description of Unknown Bacteria <Prokaryote> sequence
  NT123 N-terminus
MKKTTALFLLIFSLIAC

SEQ ID NO: 112 - protein, 26 aa, Unknown
  Description of Unknown Bacteria <Prokaryote> sequence
  Immunostimulatory oligonucleotide
ICICICICICICICICICICICICIC

SEQ ID NO: 113 - protein, 11 aa, Unknown
  Description of Unknown Bacteria <Prokaryote> sequence
  Polycationicoligopeptide
KLKLLLLLKLK

SEQ ID NO: 114 - protein, 122 aa, Unknown
  Description of Unknown Bacteria <Prokaryote> sequence
  NT119
DTLEQQFQQGSEATTRGDYQTTFKFLLPLAEQGNAEAQLMLGVMYARGIGVKQDDFEAVKWYRQAAEQGYANAQAILGFSYLLGQSGVQVNKSLAKEWFGKACDNGDQNGCEYYGKLNRGEL

SEQ ID NO: 115 - protein, 158 aa, Unknown
  Description of Unknown Bacteria <Prokaryote> sequence
  NT120
DTLEQQFQQGLTAYEQSNYQTAFKLWLPMAEQGYAKAQFNLGVMYAKGQGVKQDDFEAVKWFRKAAEQGYAEAKFNLGHMYSKGRGVKQDDFEAVNWYRKAAEQGDADAQAILGFLYLLGERGVQVNKSLAKEWFGKACDNGNQNGCEYYGKLNRGEL

SEQ ID NO: 116 - protein, 157 aa, Unknown
  Description of Unknown Bacteria <Prokaryote> sequence
  NT121
DTLEQQFQQGLTAYEQSNYQTAFKLWLPLAEQGDANVQFNLGVMYAEGQGVKQDDFEAVKWYRKAAEQGDANAQAYLGLAYTEGRGVRQDYTEAVKWFRKAAEQGHANAQAILGFSYLLGKGVQVNKSLAKEWFGKACDNGDQGGCKYYGKLNRGER

SEQ ID NO: 117 - protein, 168 aa, Unknown
  Description of Unknown Bacteria <Prokaryote> sequence
  NT122
FDKQEAKQKVEDTKQTVASVASETKDAAANTMTEVKEKAQQLSTDVKNKVAEKVEDAKEVIKSATETASEKATEIKEAVSEKASEMKEAASEKASEMKEAASEKASEMKEAASEKASEMKEAASEKASEMKEAASEKVGEMKEKATEMKEAVSEKATQAVDAVKEATK

SEQ ID NO: 118 - protein, 26 aa, Unknown
  Description of Unknown Bacteria <Prokaryote> sequence
  NT119encoded N-terminus
MRFTKTLFTTALLGASIFSFQSTAWA

SEQ ID NO: 119 - protein, 26 aa, Unknown
  Description of Unknown Bacteria <Prokaryote> sequence
  NT120encoded N-terminus
MKLTKTLLTTALFGASVFSFQSTAWA

SEQ ID NO: 120 - protein, 26 aa, Unknown
  Description of Unknown Bacteria <Prokaryote> sequence
  NT121encoded N-terminus
MKLTKTLLTTALLGASVFSFQSTAWA

SEQ ID NO: 121 - protein, 23 aa, Unknown
  Description of Unknown Bacteria <Prokaryote> sequence
  NT122encoded N-terminus
MEKIMKKLTLALVLGSALAVTGC

SEQ ID NO: 122 - protein, 173 aa, Unknown
  Description of Unknown Bacteria <Prokaryote> sequence
  NT004
SEEQVQRDVYQSLDDCLADWKKIELCEADKNTESTQKTETTPQQGLGLNIRDNGNAESAVKNPAENNVQANQSENNAESTTKAESTDPSLGAAIAGGVMGYLAARAISSFLGPSYHPGNRAVTTPTGQVVQPQTNRSVGKPMLVKGNAGSMNSKPVSRGGFSSPNNTHRSSGG

SEQ ID NO: 123 - protein, 171 aa, Unknown
  Description of Unknown Bacteria <Prokaryote> sequence
  NT014
VAAVIGGGAVAAKVATDPRTTGTQIDDETLEFKVENAVEKDAQIKAEGRVNAVSYNGRVLLIGQVPNSDVKDTATALAKGVKGVNEVYNELTVSSKISFAQISKDSWLTTQVKSKMFVDGRVKATDVKVISENGEVFLLGNVTQSQANAAADIASKISGVKKVIKVFKYLD

SEQ ID NO: 124 - protein, 387 aa, Unknown
  Description of Unknown Bacteria <Prokaryote> sequence
  NT022
TSNFPAPISDADGNLSPSVIQSVNGSNVGGAWQPEIQKNSLPTTGNMVTPQPNFQPINQQPTMPTAPAQPAFQPSPKTVVSAPTVQTKTVTKTVADCVDGQHINIPRNPNTNVPDYSKISKGSYKGNTYKVNKGDTMFLIAYLAGIDVKELAALNNLSEPYNLSLGQVLKISNCSTKTVTTTVSVKQPAVTTSTATPVKPAVTYTPGANGTQIGSDGTIIGPIKSEAGTSPSVPVATSSTQVTSSVNNANSTPINSNVVAPIASHVVWQWPTSGNIIQGFSSTDGGNKGIDISGSRGQAVKAAAAGRIVYAGNALRGYGNLIIIKHNDDFLSAYAHNDKILVADQQEVKAGQDIAKMGSSGTNTVKLHFEIRYKGKSVDPVRYLPRH

SEQ ID NO: 125 - protein, 20 aa, Unknown
  Description of Unknown Bacteria <Prokaryote> sequence
  NT004 N-terminus
MKKKNQILVSLSIVALLGGC

SEQ ID NO: 126 - protein, 22 aa, Unknown
  Description of Unknown Bacteria <Prokaryote> sequence
  NT014 N-terminus
MTLSPLKKLAILLGATIFLQGC

SEQ ID NO: 127 - protein, 22 aa, Unknown
  Description of Unknown Bacteria <Prokaryote> sequence
  NT022 N-terminus
MTLSPLKKLAILLGATIFLQGC

SEQ ID NO: 128 - protein, 286 aa, Artificial Sequence
  Description of Artificial Sequence Synthetic polypeptide
  NT061
EERVVATVDGIPILESQVRANMGKKGDRQSALDKIIDDLLVQKAIQESGVKIDPREIDRVVEDTAARNGLTYGQFLDALDYQGISLNTFRQQIANQMVMGAVRNKAIQESIDVTREEVVALGQKMLDEAKSQGTAQKVTGKEYEVRHILLKLNPLLNDAQAKKQLAKIRSDIIAGKTTFADAALKYSKDYLSGANGGSLGYAFPETYAPQFAQTVMKSKQGVISAPFKTEFGWHILEVTGVSDGDLTAEAYTQKAYERLVNTQLQDATNDWVKALRKRANIQYFNK

SEQ ID NO: 129 - protein, 27 aa, Artificial Sequence
  Description of Artificial Sequence Synthetic peptide
  NT061 N-terminus
MKMKKFILKSFLLATLGCVAFTSMAQA

SEQ ID NO: 130 - protein, 390 aa, Artificial Sequence
  Description of Artificial Sequence Synthetic polypeptide
  NT017
GLLIFSPVSQSSDLNQIQKQIKQQESKIEKQKREQAKLQANLKKHESKINTVEGELLETEISLKEIRKQIADADKQFKQLEKQEREQKARLAKQMDIIYRSGINPSLIERMFAQDPTKAERMKVYYQHLNQVRIEMIDNLKATQAQIAVQKEAILAQQKNHRNQLSTQKKQQQALQKAQQEHQSTLNELNKNLALDQDKLNALKANEQALRQEIQRAEQAAREQEKREREALAQRQKAEEKRTSKPYQPTVQERQLINSTSGLGAAKKQYSLPVSGSILHTFGSIQAGEVRWKGMVIGASAGTPVKAIAAGRVILAGYLNGYGYMVIVKHGETDLSLYGFNQAVSVKVGQLVSAGQVIAQVGNTGEISRSALYFGISRKGTPVNPAGWVR

SEQ ID NO: 131 - protein, 20 aa, Artificial Sequence
  Description of Artificial Sequence Synthetic peptide
  NT017 N-terminus
MLRFGVNQKTSLLLTALLSC